SEQUENCE LISTING (90 sequences)

SEQ ID NO: 1 - DNA, 20 bp, Artificial Sequence
  HSV-2 gD Forward Primer
agcgaggataacctgggatt

SEQ ID NO: 2 - DNA, 20 bp, Artificial Sequence
  HSV-2 gD Reverse Primer
gggataaagcggggtaacat

SEQ ID NO: 3 - DNA, 19 bp, Artificial Sequence
  VSV Forward Primer
acggcgtacttccagatgg

SEQ ID NO: 4 - DNA, 19 bp, Artificial Sequence
  VSV Reverse Primer
ctcggttcaagatccaggt

SEQ ID NO: 5 - protein, 12 aa, Artificial Sequence
  EBV BHRF1
TVVLRYHLLEEY

SEQ ID NO: 6 - protein, 18 aa, Artificial Sequence
  EBV BALF1
AGLTLSLLVICSYLFISR

SEQ ID NO: 7 - protein, 11 aa, Artificial Sequence
  EBV BYRF1
TVFYNIPPMPL

SEQ ID NO: 8 - protein, 13 aa, Artificial Sequence
  EBV EBNA1 P1
TSLYNLRRGTALA

SEQ ID NO: 9 - protein, 15 aa, Artificial Sequence
  EBV EBNA1 P2
LDLDFGQLTPHTKAV

SEQ ID NO: 10 - protein, 15 aa, Artificial Sequence
  EBV BZLF1 (11-25)
VKFTPDPYQVPFVQA

SEQ ID NO: 11 - protein, 15 aa, Artificial Sequence
  EBV BZLF1 (61-75)
LTAYHVSTAPTGSWF

SEQ ID NO: 12 - protein, 15 aa, Artificial Sequence
  EBV BZLF1 (116-130)
PGDNSTVQTAAAVVF

SEQ ID NO: 13 - protein, 15 aa, Artificial Sequence
  EBV BZLF1 (407-421)
PPVKRKKGLRDSREG

SEQ ID NO: 14 - protein, 15 aa, Artificial Sequence
  EBV BMLF1 (41-55)
DEDPTPAHAIPARPS

SEQ ID NO: 15 - protein, 12 aa, Artificial Sequence
  EBV BMHRF1 (122-133)
PYYVVDLSVRGM

SEQ ID NO: 16 - protein, 15 aa, Artificial Sequence
  EBV BMRF1 (136-150)
VKLTMEYDDKVSKSH

SEQ ID NO: 17 - protein, 20 aa, Artificial Sequence
  EBV BKRF2 (116-135)
GSFSVEDLFGANLNRYAWHR

SEQ ID NO: 18 - protein, 20 aa, Artificial Sequence
  EBV BKZLF2 (186-205)
QIFGSHCTYVSKFSTVPVSH

SEQ ID NO: 19 - protein, 20 aa, Artificial Sequence
  EBV BLLF1 (61-81)
LDLDFGQLTPHTKAVYQPRG

SEQ ID NO: 20 - protein, 15 aa, Artificial Sequence
  EBV BALF4 (575-589)
DNEIFLTKKMTEVCQ

SEQ ID NO: 21 - protein, 20 aa, Artificial Sequence
  EBV BXLF2 (126-140)
LEKQLFYYIGTMLPNTRPHS

SEQ ID NO: 22 - protein, 13 aa, Artificial Sequence
  FLU HLA-DRA*01
PKYVKQNTLKLAT

SEQ ID NO: 23 - protein, 13 aa, Artificial Sequence
  FLU NP DRB1*0901
VFELSDEKAASPI

SEQ ID NO: 24 - protein, 13 aa, Artificial Sequence
  FLU NP DRB1*0404
GQISIQPTFSVQR

SEQ ID NO: 25 - protein, 11 aa, Artificial Sequence
  FLU M1 DPB1*0301
RQMVQAMRTIG

SEQ ID NO: 26 - protein, 13 aa, Artificial Sequence
  FLU M1 DRB1*0101
GLIYNRMGAVTTE

SEQ ID NO: 27 - protein, 13 aa, Artificial Sequence
  FLU M1 (43-55)
MEWLKTRPILSPL

SEQ ID NO: 28 - protein, 13 aa, Artificial Sequence
  FLU M1 (94-106)
DKAVKLYRKLKRE

SEQ ID NO: 29 - protein, 14 aa, Artificial Sequence
  FLU M1 (101-113)
RKLKREITFHGAKK

SEQ ID NO: 30 - protein, 13 aa, Artificial Sequence
  FLU M1 (105-117)
GLIYNRMGAVTTE

SEQ ID NO: 31 - protein, 14 aa, Artificial Sequence
  FLU M1 (209-221)
ARQMVQAMRTIGTT

SEQ ID NO: 32 - protein, 14 aa, Artificial Sequence
  FLU NP (102-114)
GKWMRELILYDKER

SEQ ID NO: 33 - protein, 13 aa, Artificial Sequence
  FLU NP (115-127)
EIRRIWRQANNGD

SEQ ID NO: 34 - protein, 13 aa, Artificial Sequence
  FLU NP (404-416)
GQISIQPTFSVQR

SEQ ID NO: 35 - protein, 13 aa, Artificial Sequence
  FLU NP (463-475)
VFELSDEKAASPI

SEQ ID NO: 36 - protein, 17 aa, Artificial Sequence
  FLU H2 HA339-355
SRGLFGAIAGFIEGGWQ

SEQ ID NO: 37 - protein, 17 aa, Artificial Sequence
  FLU H1 HA340-356
IQSRGLFGAIAGFIEGG

SEQ ID NO: 38 - protein, 17 aa, Artificial Sequence
  FLU H3 HA338-354
NVPEKQTRGIFGAIAGF

SEQ ID NO: 39 - protein, 17 aa, Artificial Sequence
  FLU H3 HA344-360
TRGIFGAIAGFIENGWE

SEQ ID NO: 40 - protein, 17 aa, Artificial Sequence
  FLU H5 HA345-361
KRGLFGAIAGFIEGGWQ

SEQ ID NO: 41 - protein, 17 aa, Artificial Sequence
  FLU B/HA360-376
ERGFFGAIAGFLEGGWE

SEQ ID NO: 42 - protein, 17 aa, Artificial Sequence
  FLU NS1-11
ESDEAFKMTMASALASR

SEQ ID NO: 43 - protein, 17 aa, Artificial Sequence
  FLU NS1-21
LTLLRAFTEEGAIVGEI

SEQ ID NO: 44 - protein, 17 aa, Artificial Sequence
  FLU NS1-22
FTEEGAIVGEISPLPSL

SEQ ID NO: 45 - protein, 17 aa, Artificial Sequence
  FLU NPp74
SDMRAEIIKMMESARPE

SEQ ID NO: 46 - protein, 17 aa, Artificial Sequence
  FLU NPp75
IIKMMESARPEEVSFQG

SEQ ID NO: 47 - protein, 20 aa, Artificial Sequence
  FLU HA33-52
VDTVLEKNVTVTHSVNLLED

SEQ ID NO: 48 - protein, 20 aa, Artificial Sequence
  FLU HA113-132
IDYEELREQLSSVSSFERFE

SEQ ID NO: 49 - protein, 20 aa, Artificial Sequence
  FLU HA121-140
QLSSVSSFERFEIFPKTSSW

SEQ ID NO: 50 - protein, 20 aa, Artificial Sequence
  FLU HA161-180
FYKNLIWLVKKGNSYPKLSK

SEQ ID NO: 51 - protein, 20 aa, Artificial Sequence
  FLU HA249-268
TLVEPGDKITFEATGNLVVP

SEQ ID NO: 52 - protein, 20 aa, Artificial Sequence
  FLU HA257-276
ITFEATGNLVVPRYAFAMER

SEQ ID NO: 53 - protein, 20 aa, Artificial Sequence
  FLU HA265-284
LVVPRYAFAMERNAGSGIII

SEQ ID NO: 54 - protein, 20 aa, Artificial Sequence
  FLU HA305-324
TSLPFQNIHPITIGKCPKYV

SEQ ID NO: 55 - protein, 13 aa, Artificial Sequence
  FLU HA307-319
PKYVKQNTLKLAT

SEQ ID NO: 56 - protein, 21 aa, Artificial Sequence
  CMV pp65 108-127
MSIYVYALPLKMLNIPSINVH

SEQ ID NO: 57 - protein, 14 aa, Artificial Sequence
  CMV pp65 115-127
LPLKMLNIPSINVH

SEQ ID NO: 58 - protein, 12 aa, Artificial Sequence
  CMV pp65 14-25
VLGPISGHVLKA

SEQ ID NO: 59 - protein, 11 aa, Artificial Sequence
  CMV pp65 47-57
HVRVSQPSLIL

SEQ ID NO: 60 - protein, 13 aa, Artificial Sequence
  CMV pp65 337-349
VELRQYDPVAALF

SEQ ID NO: 61 - protein, 18 aa, Artificial Sequence
  CMV pp65 370-389
IVKPGDILVINSNGNLIA

SEQ ID NO: 62 - protein, 18 aa, Artificial Sequence
  CMV pp65 494-511
RNLVPMVATVQGQNLKYQ

SEQ ID NO: 63 - protein, 14 aa, Artificial Sequence
  CMV pp65 518-531
NDIYRIFAELEGVW

SEQ ID NO: 64 - protein, 15 aa, Artificial Sequence
  CMV DR15 (DQ6)
LLQTGIHVRVSQPSL

SEQ ID NO: 65 - protein, 15 aa, Artificial Sequence
  CMV DR53 (DQ3)
IIKPGKISHIMLDVA

SEQ ID NO: 66 - protein, 15 aa, Artificial Sequence
  CMV DR11 P1
PQYSEHPTFTSQYRI

SEQ ID NO: 67 - protein, 15 aa, Artificial Sequence
  CMV Kern 2005
EHPTFTSQYRIQGKL

SEQ ID NO: 68 - protein, 15 aa, Artificial Sequence
  CMV Kern 2006
FTSQYRIQGKLEYRH

SEQ ID NO: 69 - protein, 15 aa, Artificial Sequence
  CMV DR52
QEFFWDANDIYRIFA

SEQ ID NO: 70 - protein, 15 aa, Artificial Sequence
  CMV Kern 2008
FFWDANDIYRIFAEL

SEQ ID NO: 71 - protein, 15 aa, Artificial Sequence
  CMV Kern 2009
AGILARNLVPMVATV

SEQ ID NO: 72 - protein, 16 aa, Artificial Sequence
  CMV Kern 2010
PPWQAGILARNLVPMV

SEQ ID NO: 73 - protein, 15 aa, Artificial Sequence
  CMV Kern 2011
ARNLVPMVATVQGQN

SEQ ID NO: 74 - protein, 15 aa, Artificial Sequence
  CMV DR15 P1
LLQTGIHVRVSQPSL

SEQ ID NO: 75 - protein, 15 aa, Artificial Sequence
  CMV DR1/DR3
PLKMLNIPSINVHHY

SEQ ID NO: 76 - protein, 15 aa, Artificial Sequence
  CMV DR1 P1
TRQQNQWKEPDVYYT

SEQ ID NO: 77 - protein, 15 aa, Artificial Sequence
  CMV DR7
EPDVYYTSAFVFPTK

SEQ ID NO: 78 - protein, 15 aa, Artificial Sequence
  CMV DR15 P2
KVYLESFCEDVPSGK

SEQ ID NO: 79 - protein, 15 aa, Artificial Sequence
  CMV DR3 P1
TLGSDVEEDLTMTRN

SEQ ID NO: 80 - protein, 15 aa, Artificial Sequence
  CMV DR13
QPFMRPHERNGFTVL

SEQ ID NO: 81 - protein, 15 aa, Artificial Sequence
  CMV DR4/DR7
IIKPGKISHIMLDVA

SEQ ID NO: 82 - protein, 15 aa, Artificial Sequence
  CMV DR11 P2
EHPTFTSQYRIQGKL

SEQ ID NO: 83 - protein, 15 aa, Artificial Sequence
  CMV DR3 P2
YRIQGKLEYRHTWDR

SEQ ID NO: 84 - protein, 15 aa, Artificial Sequence
  CMV DR14
TERKTPRVTGGGAMA

SEQ ID NO: 85 - protein, 15 aa, Artificial Sequence
  CMV DR11 P3
ASTSAGRKRKSASSA

SEQ ID NO: 86 - protein, 15 aa, Artificial Sequence
  CMV DR1 P2
ACTSGVMTRGRLKAE

SEQ ID NO: 87 - protein, 15 aa, Artificial Sequence
  CMV DR11 DR3
AGILARNLVPMVATV

SEQ ID NO: 88 - protein, 15 aa, Artificial Sequence
  CMV DR1 DR3
KYQEFFWDANDIYRI

SEQ ID NO: 89 - protein, 47 aa, Artificial Sequence
  HSV RVG-gDT
YTIWMPENPRPGTPCDIFTNSRGKRASNGGGGCCIPPNWHIPSIQDA

SEQ ID NO: 90 - protein, 13 aa, Artificial Sequence
  HSV gD315-327
IPPNWHIPSIQDA

(19) United States
(12) Patent Application Publication
(10) Pub. No.: US 2023/0029362 A1
(43) Pub. Date: Jan. 26, 2023

(54) A T cell-based immunotherapy for central nervous system viral infections and tumors

(71) Applicant: YALE UNIVERSITY, New Haven, CT (US)

(72) Inventor: Akiko Iwasaki, New Haven, CT (US)

(21) Appl. No.: 17/782,338

(22) PCT Filed: Dec. 4, 2020
(86) PCT No.: PCT/US20/63211

§ 371 Date: Jun. 3, 2022

Related U.S. Application Data
(60) Provisional application No. 62/943,930, filed on Dec. 5, 2019.

(51) Int. Cl. A61K 39/00; A61K 39/12; A61K 39/395; C07K 16/10

(52) U.S. Cl. CPC A61K 39/001114; A61K 39/12; A61K 39/39558; C07K 16/1018; A61K 2039/505; C07K 2317/76

(57) ABSTRACT

The present disclosure relates to compositions and methods for treating or preventing a disease or disorder of the brain, spinal cord or central nervous system. It is described herein that an immunogenic composition which induces a CD4 T cell immune response induces permeability of the blood brain barrier, and allows for the access of a therapeutic antibody or agent to the brain, spinal cord or central nervous system.

### CROSS-REFERENCE TO RELATED APPLICATIONS

This application claims priority to U.S. Provisional Patent Application No. 62/943,930, filed Dec. 5, 2019, the contents of which are incorporated by reference herein in its entirety.

### STATEMENT REGARDING FEDERALLY SPONSORED RESEARCH OR DEVELOPMENT

This invention was made with government support under AI054359, AI062428, AI064705, AI054359, AI127429, and F30CA239444 awarded by the National Institutes of Health (NIH). The government has certain rights in the invention.

### BACKGROUND OF THE INVENTION

Vesicular stomatitis virus (VSV) is a negative sense RNA neurotropic virus, closely related to rabies virus. In murine models, the route of VSV exposure influences pathogenesis, host response and subsequent clinical manifestations (Cuevas et al., 2017, Nat Microbiol, 2:17078). For instance, intravenous (i.v) VSV infections are well tolerated by wild-type mouse strains, generating robust memory responses (Iijima and Iwasaki, 2016, Nature, 533:552-556). In contrast, high dose intranasal (i.n) VSV delivery leads to viral dissemination to the brain. VSV infects olfactory sensory neurons in the nasal epithelium (Lundh et al., 1987, Neuropathol Appl Neurobiol, 13:11-122) and enters the central nervous system (CNS) moving along the axons to the olfactory bulb (Reiss et al., 1998, Ann N Y Acad Sci, 855:751-761). Previous studies have shown that i.n. VSV infection of mice often leads to breakdown of the blood-brain barrier (BBB) (Iijima and Iwasaki, 2016, Nature, 533:552-556; Bi et al., 1995, J Virol, 69:6466-6472). In vaccination strategies, modulation of BBB permeability is crucial to allow antibodies or protective immune cells to mediate viral resistance in the CNS.

There is thus a need in the art for compositions and methods for treating and preventing infection of the central nervous system. The present invention addresses this unmet need in the art.

### SUMMARY OF THE INVENTION

In some embodiments, the invention relates to a method for treating or preventing a disease or disorder of the brain, central nervous system or spinal cord in a subject in need thereof, comprising a) administering an immunogenic agent to induce an immune response, thereby inducing permeability of the blood brain barrier (BBB) in the subject; and b) administering at least one therapeutic agent for the treatment of the disease or disorder.

In some embodiments, the immunogenic agent is an antigenic protein or peptide for inducing a CD4 T cell immune response. In some embodiments, the immunogenic agent comprises an antigenic MHC Class II peptide. In some embodiments, the immunogenic agent comprises a peptide selected from the group consisting of SEQ ID NO:5 to SEQ ID NO:90.

In some embodiments, at least one therapeutic agent comprises an inhibitor of an immune checkpoint protein. In some embodiments, the immune checkpoint protein is PD-1, PDL-1 CTLA-4, LAG-3, TIM-3, TIGIT or CEACAM1. In some embodiments, the inhibitor is ipilimumab, nivolumab, pembrolizumab, pidilizumab, atezolizumab, BMS-986016, BMS-936559, MPDL3280A, MDX1105-01, MEDI4736, TSR-022, CM-24 or MK-3475.

In some embodiments, the disease or disorder comprises a pathogen-mediated infection selected from the group consisting of: a viral infection, a bacterial infection, a fungal infection, a protozoan infection, a prion infection, and a helminth infection.

In some embodiments, the method treats or prevents infection-associated inflammation.

In some embodiments, the method treats or prevents an infection-associated condition selected from the group consisting of: encephalitis, meningitis, meningoencephalitis, epidural abscess, subdural abscess, brain abscess, and progressive multifocal leukoencephalopathy (PML).

In some embodiments, the method treats or prevents cancer. In some embodiments, the therapeutic agent comprises an antibody or antibody fragment that specifically binds a tumor-specific or tumor-associated antigen.

In some embodiments, the invention relates to a composition for treating or preventing a disease or disorder of the brain, central nervous system or spinal cord in a subject in need thereof, comprising a) an antigenic protein or peptide to induce a CD-4 T cell immune response in the subject, thereby inducing permeability of the BBB; and b) at least one therapeutic agent for the treatment of the disease or disorder.

In some embodiments, the antigenic protein or peptide comprises an antigenic MHC Class II peptide. In some embodiments, the antigenic protein or peptide is selected from the group consisting of SEQ ID NO:5 to SEQ ID NO:90.

In some embodiments, at least one therapeutic agent comprises an inhibitor of an immune checkpoint protein. In some embodiments, the immune checkpoint protein is PD-1, PDL-1 CTLA-4, LAG-3, TIM-3, TIGIT or CEACAM1. In some embodiments, the inhibitor is ipilimumab, nivolumab, pembrolizumab, pidilizumab, atezolizumab, BMS-986016, BMS-936559, MPDL3280A, MDX1105-01, MEDI4736, TSR-022, CM-24 or MK-3475.

In some embodiments, the therapeutic agent comprises an antibody or antibody fragment that binds to an antigen associated with the disease or disorder. In some embodiments, the disease or disorder is a viral infection, a bacterial infection, a fungal infection, a protozoan infection, a prion infection, a helminth infection, encephalitis, meningitis, meningoencephalitis, epidural abscess, subdural abscess, brain abscess, progressive multifocal leukoencephalopathy (P-L), or cancer.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1, comprising FIG. 1A through FIG. 1G, is a set of images depicting the results of experiments demonstrating that intranasal immunization confers B-cell-dependent neuron protection following genital HSV-2 challenge. FIG. 1A through FIG. 1D: C57/BL6 mice were immunized with TK-HSV-2 (105 plaque-forming units (p.f.u.)) via intranasal (i.n.; n=12), intraperitoneal (i.p.; n=5) or intravaginal (ivag.; n=11) routes. Five to 6 weeks later, these mice and naive mice (n=4) were challenged with a lethal dose of WT HSV-2 (104 p.f.u.). Mortality (FIG. 1A), clinical score (FIG. 1B) and virus titer in vaginal wash (FIG. 1C) were measured on indicated days after challenge. FIG. 1D: Six days after challenge, virus titer in tissue homogenates including DRG and spinal cord was measured. FIG. 1E through FIG. 1G: BALB/c mice (n=10) or B-cell-deficient JHD mice (n=6) were immunized intranasally with TK-HSV-2 (5×10⁴ p.f.u.). Six weeks later, these mice and naive mice (n=4) were challenged with lethal WT HSV-2 (10⁵ p.f.u.). Mortality (FIG. 1E) and clinical score (FIG. 1F) were measured. FIG. 1G: Six days after challenge, virus titer in tissue homogenates including DRG and spinal cord was measured by plaque assay. Data are means±s.e.m. *P<0.05; **P<0.01; ***P<0.001; ****P<0.0001 (two-tailed unpaired Student's t-test).

FIG. 2, comprising FIG. 2A through FIG. 2G, is a set of images depicting the results of experiments demonstrating antibody-mediated neuroprotection on CD4 T cells but not on FcRn-mediated transport. FIG. 2A and FIG. 2B: C57/BL6 (WT) mice (n=4) and FcRn^{-/-} (n=10) mice were immunized intranasally with TK-HSV-2 (105 p.f.u.), and 6 weeks later challenged with a lethal dose of WT HSV-2 (104 p.f.u.). Mortality (FIG. 2A) and clinical score (FIG. 2B) were measured. FIG. 2C and FIG. 2D: μMT mice were immunized with TK-HSV-2 (105 p.f.u.) intranasally. Five to 6 weeks later, naive mice (n=3), naive mice receiving immune serum intravenously (n=4), μMT mice (n=23) and μMT mice receiving immune serum intravenously (n=10) were challenged with a lethal dose of WT HSV-2, and mortality (FIG. 2C) and clinical score (FIG. 2D) were assessed. Immune serum prepared from mice immunized 4 weeks previously with TK-HSV-2 (200 μl per mouse) was injected 3 h before challenge, and 3 and 6 days after challenge. FIG. 2E & FIG. 2F: WT C57/BL6 mice (n=5) and IFN-γR^{-/-} mice (n=8) immunized intranasally with TK-HSV-2 (10⁵ p.f.u.) 6 weeks previously were challenged with a lethal dose of WT HSV-2, and mortality (FIG. 2E) and clinical score (FIG. 2F) were assessed. Depletion of CD4 T cells (n=4) or neutralization of IFN-γ (n=5) was performed on days −4, −1, 2 and 4 before/after challenge by intravenous injection of anti-CD4 (GK1.5) or anti-IFN-γ (XMG1.2), respectively. FIG. 2G: Six days after challenge, virus titer in tissue homogenates including DRG and spinal cord was measured by plaque assay (FIG. 2E). Data are means±s.e.m. *P<0.05; **P<0.01 (two-tailed unpaired Student's t-test).

FIG. 3, comprising FIG. 3A through FIG. 3D, is a set of images depicting the results of experiments demonstrating that memory of CD4+ T cells are required for antibody access to neuronal tissues. Naive WT mice or WT and μMT mice intranasally immunized with TK-HSV-2 (105 p.f.u.) 6 weeks earlier were challenged with a lethal dose of WT HSV-2 intravaginally. Six days after the challenge, after extensive perfusion, HSV-2-specific (FIG. 3A, FIG. 3C) and total Ig (FIG. 3B, FIG. 3D) levels in tissue homogenates of DRG and spinal cord were analyzed by ELISA. To deplete CD4 T cells, CD4-specific antibody was injected on days −4, −1, 2 and 4 before/after challenge. Data are means±s.e.m. *P<0.05; **P<0.01; ***P<0.001 (two-tailed unpaired Student's t-test).

FIG. 4, comprising FIG. 4A through FIG. 4F, is a set of images depicting the results of experiments demonstrating that α4-Integrin-dependent recruitment of memory CD4+ T cells required for antibody access to neuronal tissues. WT mice immunized intranasally with TK-HSV-2 6 weeks earlier were challenged with a lethal dose of WT HSV-2. Neutralization of α4-integrin was performed on days 2 and 4 after challenge by intravenous injection of anti-α4 integrin (CD49d) antibody. FIG. 4A: Six days after challenge, after extensive perfusion, HSV-2-specific IFN-γ⁺ CD4⁺ T cells in DRG and spinal cord were detected by flow cytometry. FIG. 4B: The number of IFN-γ-secreting CD4 T cells among 50,000 cells of CD45^{hi} leukocytes in DRG and spinal cord is depicted. Data are means±s.e.m. *P<0.05; **P<0.01; ***P<0.001 (two-tailed unpaired Student's t-test). FIG. 4C: Frozen sections of DRG were stained with antibodies against CD4, VCAM-1 or CD31. Nuclei are depicted by 4′,6-diamidino-2-phenylindole (DAPI) stain (blue). Images were captured using a ×10 or ×40 objective lens. Scale bars, 100 μm. Arrowhead indicates VCAM-1⁺ cells in parenchyma of DRG. Data are representative of at least three similar experiments. HSV-2-specific antibodies in the DRG (FIG. 4D) and spinal cord (FIG. 4E) were analyzed by ELISA. Data are means±s.e.m. *P<0.05 (two-tailed paired Student's t-test) Albumin level in tissue homogenates was analyzed by ELISA (FIG. 4F). Depletion of CD4 T cells or neutralization of IFN-γ was performed on days −4, −1, 2 and 4 before/after challenge by intravenous injection of anti-CD4 (GK1.5) or anti-IFN-γ (XMG1.2), respectively. Data are means±s.e.m. *P<0.05; **P<0.01; ***P<0.001 (two-tailed paired Student's t-test).

FIG. 5, comprising FIG. 5A through FIG. 5H, is a set of images depicting the results of experiments demonstrating that in the absence of TRM, B cells are required for the protection of the host against genital HSV-2 challenge. FIG. 5A: C57BL/6 mice and μMT mice were immunized intravaginally or intranasally with TK-HSV-2. Five weeks later, vaginal tissue sections were stained for CD4⁺ cells (red) and MHC class II⁺ cells (green). Blue labelling depicts nuclear staining with DAPI (blue). Images were captured using a ×10 or ×40 objective lens. Scale bars, 100 μm. Data are representative of three similar experiments. FIG. 5B through Figure D: BALB/c mice and JHD mice were immunized with TK-HSV-2 (105 p.f.u.) intranasally or intravaginally. Six weeks later, the number of total CD4+ T cells and HSV-2-specific IFN-γ⁺ CD4⁺ T cells in the vagina (FIG. 5B), spleen (FIG. 5C) and peripheral blood (FIG. 5D) were analyzed by flow cytometry. Percentages and total number of IFN-γ⁺ cells among CD4⁺CD90.2⁺ cells are shown. Data are means±s.e.m. *P<0.05; **P<0.001; ***P<0.001 (two-tailed unpaired Student's t-test). FIG. 5E: C57/BL6 mice were immunized intravaginally (naive→D7) or intranasally (WT/i.n. →D0) with TK-HSV-2 virus. At the indicated time points (D7: 7 days after immunization; WT/i.n. →D0: 6 weeks after immunization), total viral genomic DNA in the vaginal tissues, DRG and spinal cord were measured by quantitative PCR. FIG. 5F-FIG. 5H: Intravaginally immunized C57BL/6 (WT), μMT and HEL-BCR Tg mice (left partner) were surgically joined with naive WT mice (right partner). Three weeks after parabiosis, the naive partner was challenged with a lethal dose of WT HSV-2 intravaginally. Mortality (FIG. 5E), clinical score (FIG. 5F) and virus titer in vaginal wash (FIG. 5G) following viral challenge are depicted.

FIG. 6, comprising FIG. 6A and FIG. 6B is a set of images depicting the results of experiments demonstrating that mucosal TK-HSV-2 immunization generates higher levels of virus-specific IgG2b and IgG2c compared with intraperitoneal immunization. WT mice were immunized with TK-HSV-2 (105 p.f.u. per mouse) via intravaginal, intraperitoneal or intranasal routes. Six weeks later, these mice were challenged with a lethal dose of WT HSV-2 intravaginally. At the indicated days after challenge, HSV-2-specific Ig (FIG. 6A) and total Ig (FIG. 6B) in serum were analyzed by ELISA. Data are means±s.e.m. *P<0.05 (Mann-Whitney U-test).

FIG. 7, comprising FIG. 7A and FIG. 7B, is a set of images depicting the results of experiments demonstrating that the enhancement of antibody access to the DRG with IFN-γ. WT mice immunized with TK-HSV-2 (105 p.f.u. per mouse) intranasally 6 weeks earlier were challenged with a lethal dose of WT HSV-2 intravaginally. Six days after challenge, after extensive perfusion, HSV-2-specific (FIG. 7A) and total Ig (FIG. 7B) in DRG homogenates were analyzed by ELISA. Depletion of CD4 T cells or neutralization of IFN-γ was performed on days −4, −1, 2 and 4 before/after challenge by intravenous injection of anti-CD4 (GK1.5) or anti-IFN-γ (XMG1.2), respectively. Data are means±s.e.m. *P<0.05; **P<0.001 (two-tailed unpaired Student's t-test).

FIG. 8, comprising FIG. 8A through FIG. 8D, is a set of images depicting the results of experiments investigating the neutralization of IFN-γ, demonstrating that α4-integrin or depletion of CD4 T cells has no impact on circulating immunoglobulin levels. FIG. 8A and FIG. 8B: WT mice immunized intranasally with TK-HSV-2 6-8 weeks earlier were challenged with a lethal dose of WT HSV-2. Depletion of CD4 T cells or neutralization of IFN-γ was performed on days −4, −1, 2 and 4 before/after challenge by intravenous injection of anti-CD4 (GK1.5) or anti-IFN-γ (XMG1.2), respectively. At time points indicated, HSV-2-specific Ig in the blood (n=4) (FIG. 8A) and total Ig in the blood (n=4) (FIG. 8B) were measured. FIG. 8C and FIG. 8D: WT mice immunized intranasally with TK-HSV-2 6 weeks earlier were challenged with a lethal dose of WT HSV-2. Neutralization of α4-integrin was performed on days 2 and 4 after challenge by intravenous injection of anti-α4-integrin/CD49b antibody. Six days later, HSV-2-specific antibody (FIG. 8C) and total antibody (FIG. 8D) in the blood were measured. Data are representative of three similar experiments.

FIG. 9, comprising FIG. 9A through FIG. 9D, is a set of images depicting the results of experiments demonstrating that an irrelevant immunization failed to increase the levels of total antibodies in neuronal tissues. FIG. 9A: C57BL/6 mice were immunized with a sublethal dose of influenza A/PR8 virus (10 p.f.u. per mouse) intranasally. Three weeks later, Flu-specific IFN-γ⁺ CD4⁺ T cells in spleen and neuronal tissues (DRG and spinal cord) (CD45.2+) following co-culture with HI-Flu/PR8 loaded splenocytes (CD45.1V) were analyzed by flow cytometry. As a control, lymphocytes isolated from spleen of TK-HSV-2 intranasally immunized mice 6 weeks after vaccination were used for co-culture. (***P<0.001; two-tailed unpaired Student's t-test). FIG. 9B through FIG. 9D: C57BL/6 mice were immunized with a sublethal dose of influenza A/PR8 virus (10 p.f.u. per mouse). Four weeks later, these mice were challenged with a lethal dose of WT HSV-2 (10⁴ p.f.u. per mouse) intravaginally. Six days after challenge, total antibodies in lysate in DRG (FIG. 9B), spinal cord (FIG. 9C) and blood (FIG. 9D) were measured by ELISA.

FIG. 10, comprising FIG. 10A and FIG. 10B, is a set of images depicting the results of experiments demonstrating that most CD4 T cells recruited to the DRG and spinal cord of immunized mice are localized in the parenchyma of neuronal tissues. FIG. 10A: C57BL/6 mice were immunized intranasally with TK-HSV-2. Six days after challenge of immunized mice 6 weeks prior, neuronal tissue sections (DRG and spinal cord) were stained for CD4⁺ cells and VCAM-1⁺ cells or CD31⁺ cells (red or green). Blue labelling depicts nuclear staining with DAPI (blue). Images were captured using a ×10 or ×40 objective lens. Scale bars, 100 μm. FIG. 10B: C57BL/6 mice were immunized intranasally with TK-HSV-2. Six weeks later, mice were challenged with WT HSV-2 intravaginal and neuronal tissues were collected 6 days later. DRG and spinal cord were stained for CD4⁺ cells (red) and MHC class II⁺ cells, CD11b⁺ cells or Ly6G⁺ cells (green). Blue labelling depicts nuclear staining with DAPI (blue). Images were captured using a ×10 or ×40 objective lens. Scale bars, 100 μm. Data are representative of at least three similar experiments.

FIG. 11, comprising FIG. 11A and FIG. 11B, is a set of images depicting the results of experiments demonstrating that intravascular staining reveals the localization of CD4 T cells in the parenchyma of neuronal tissues. FIG. 11A and FIG. 11B: C57BL/6 mice immunized intranasally with TK⁻ HSV-2 6 weeks previously were challenged with lethal WT HSV-2. Six days after challenge, Alexa Fluor 700-conjugated anti-CD90.2 antibody (3 μg per mouse) was injected intravenously (tail vain) into immunized mice. Five minutes later, these mice were killed for fluorescence-activated cell sorting analysis of intravascular versus extravascular lymphocytes. Data are representative of at least two similar experiments.

FIG. 12, comprising FIG. 12A through FIG. 12C, is a set of images depicting the results of experiments demonstrating increased epithelial and vascular permeability in vaginal tissues using recombinant IFN-γ. FIG. 12A: WT mice immunized with TK-HSV-2 (105 p.f.u.) intranasally 6 weeks earlier were injected intravaginally with recombinant mouse IFN-γ (10 μg per mouse) (n=3) or PBS (n=3). At the indicated time points, HSV-2-specific Ig (FIG. 12A) and total Ig (FIG. 12B) in vaginal wash were measured by ELISA. FIG. 12C: Two days after rIFN-γ treatment, vaginal tissue sections were stained for VCAM-1⁺ cells (red) or CD4⁺ cells (green) and CD31V cells (green). Blue labelling depicts nuclear staining with DAPI (blue). Images were captured using a ×10 or ×40 objective lens. Scale bars, 100 μm. Data are representative of at least three similar experiments.

FIG. 13, comprising FIG. 13A and FIG. 13B, is a set of images depicting the results of experiments demonstrating that vascular permeability in DRG and spinal cord is augmented following WT HSV-2 challenge. FIG. 13A: C57BL/6 mice were immunized intranasally with TK-HSV-2. Six days after challenge of mice immunized 6 weeks previously, neuronal tissue sections (DRG and spinal cord) were stained for CD4⁺ cells (red) and mouse albumin (green). Blue labelling depicts nuclear staining with DAPI (blue). FIG. 13B: C57BL/6 mice were immunized intranasally with TK-HSV-2. Six weeks later, these mice were challenged with lethal WT HSV-2. Six days after challenge, Oregon green 488-conjugated dextran (70 kDa) (5 mg ml⁻¹, 200 μl per mouse) was injected intravenously into intranasally immunized mice. Forty-five minutes later, these mice were killed for immunohistochemical analysis. GM, grey matter; WM, white matter. Data are representative of three similar experiments.

FIG. 14, comprising FIG. 14A through FIG. 14D, is a set of images depicting the results of experiments demonstrating the requirement of memory CD4⁺ T cells for the increase in antibody levels and vascular permeability in the brain following VSV immunization and challenge. FIG. 14A: C57BL/6 mice were immunized intravenously with WT VSV (2×10⁶ p.f.u. per mouse). Five weeks later, these mice were challenged intranasally with WT VSV (1×10⁷ p.f.u. per mouse). Six days after challenge, VSV-specific IFN-γ⁺ CD4⁺ T cells in spleen (CD45.2⁺) following co-culture with HI-VSV loaded splenocytes (CD45.1⁺) or HI HSV-2 loaded splenocytes were analysed by flow cytometry. Data are means±s.e.m. *P<0.05; **P<0.001 (two-tailed unpaired Student's t-test). FIG. 14B and FIG. 14C: Five weeks after VSV immunization, these mice were challenged intranasally with WT VSV (1×10⁷ p.f.u. per mouse). Six days after challenge, VSV-specific antibodies and total antibodies in lysate of brain (FIG. 14B) and serum (FIG. 14C) were measured by ELISA. Depletion of CD4 T cells was performed on days −4, −1, 2 and 4 before/after challenge by intravenous injection of anti-CD4 (GK1.5). FIG. 14D: Albumin levels in tissue homogenates were analysed by ELISA. Data are means±s.e.m. *P<0.05; *P<0.01; ***P<0.001 (Mann-Whitney U-test).

FIG. 15, comprising FIG. 15A through FIG. 15H, depicts exemplary experimental results demonstrating that VSV immunization-induced transient BBB permeability allows for efficient viral control in the CNS. C57BL/6 mice were immunized with VSV (2×10⁶ plaque-forming units (p.f.u.)) subcutaneously. Five to six weeks later, these mice (VSV-immunized) and naive mice (Non-Immunized) (n=2-9) were challenged with a lethal dose of VSV (10⁷ p.f.u.). FIG. 15A depicts data demonstrating viral RNA copies in the olfactory bulb, cerebrum and cerebellum determined by qRT-PCR at days 1, 2, 3, 4, 6 and 10 post VSV intranasal challenge. Results are expressed as RNA relative expression and normalized by HPRT. Serum albumin (FIG. 15B), mortality (FIG. 15C), total IgG in brain tissue (FIG. 15D) and VSV-specific IgG (FIG. 15E) were measured on indicated days after challenge. For FIG. 15F through FIG. 15H, AID sIgM DKO mice were immunized with VSV (2×10⁶ plaque-forming units (p.f.u.)) subcutaneously. Five to six weeks later, these mice (AIDsIgGM Imm) and naive mice (AIDsIgGM N.Imm) (n=6-10) were challenged with a lethal dose of VSV (10⁷ p.f.u.). FIG. 15F depicts the mortality monitored for 20 days post challenge. BC57BL/6 mice VSV-immunized (WT Imm.) and Non-Immunized mice (WT N.Imm) were used as experimental controls. Five to 6 weeks later, VSV-Immunized or Non-Immunized mice were injected intraperitonially with 5 μg/500 ul/mouse of VSV mAb or VSV Immune serum at days −1, 1 and 3 before/after intranasal VSV challenge. An IgG2a isotype control antibody was used as control. FIG. 15G depicts the viral RNA copies in the olfactory bulb, cerebrum and cerebellum determined by qRT-PCR at day 6 post VSV intranasal challenge. Flow cytometry was performed on brain-isolated leukocytes from C57BL/6 or AID sIgM DKO that were either VSV-Immunized (WT Imm. and AIDsIgGM Imm, respectively) and not immunized (WT N.Imm.). Uninfected wild type (WT NI) and C57BL/6 primary VSV infected mice but not VSV immunized (WT VSV primary) were used as controls. For cytokines detection, brain leukocytes were restimulated in vitro with P/MA and ionomycin in the presence of brefeldin A for 10-12 hours. FIG. 15H depicts the frequency of IFNγ among CD4+ T cells.

FIG. 16, comprising FIG. 16A through FIG. 16F, depicts exemplary experimental data demonstrating that local TCR-specific HSV-2 antigenic peptides delivery opens the BBB and allows for efficient antibody influx to the CNS. FIG. 16A-FIG. 16F: WT mice were immunized with TK-HSV-2 (2×10⁶ p.f.u.) subcutaneously or received an adoptive transfer of HSV-2 specific CD4 T cells (10⁶ cells/Mouse/i.v.) from gDTII-specific-DsRed transgenic donor mice. Five to six weeks after primary HSV-2 infection or 24h post cell transfer, these mice were inoculated intranasally with 1 dose of gDTII, RVG-gDTII or RVG-OVA peptides (100 μg/mouse in 10 μl, 5 μl each nostril). FIG. 16A depicts a schematic representation of HVS-2 immunization or adoptive transfer protocol. Four days after peptides administration, 1K-immunized mice were injected (retro-orbital) with 488-conjugated dextran. After 1 hour, the brains were harvested, and immunohistochemistry analyses were performed as described. In FIG. 16B, frozen sections of brain tissue after dextran injection were stained with antibodies against CD31. Nuclei are depicted by DAPI stain. Quantification of serum albumin (FIG. 16C) and total IgG (FIG. 16D) in the brain tissue 4 days post peptides treatment of TK-immunized or CD4 T cell adoptive transfer recipient mice. TK-immunized mice were injected intraperitonially with a goat anti-mouse IgG at day 3, 4 and 5 post peptides administration. At day 6 mice were injected i.v. with Qtracker 565 for vascular staining and after 2 hours the brains were harvested and analyzed. FIG. 16E shows a frozen section of olfactory bulb stained with a donkey anti-goat IgG. Nuclei are depicted by DAPI stain. Flow cytometry was performed on brain isolated leucocytes from TK-immunized or CD4 T cell adoptive transfer recipient mice. For cytokines detection cell suspensions from brain tissues of HSV-2- immunized mice were stimulated in the presence of 5 μg/ml Brefeldin A with naive splenocytes (CD45.1+CD45.2+) loaded with HSV-2 antigen (0.5 pfu equivalent per cell) for 10-12 hours. For unspecific stimulation, brain cells isolated from gDTII-transferred recipient mice were incubated with PMA and ionomycin in the presence of Brefeldin A X1 for 4 hours. FIG. 16F corresponds to the number of IFN-γ-secreting CD4 T cells among CD45hi leukocytes in brain tissues 4 days post peptides administration. The bottom panel depicts an exemplary frozen section of brain tissue from gDTII-DsRed-transferred recipient mice. Sections were stained with antibodies against CD31. Nuclei are depicted by DAPI stain.

FIG. 17, comprising FIG. 17A through FIG. 17C, depicts exemplary experimental data demonstrating that local TCR-specific viral antigenic peptides delivery boosts heterologous anti-viral responses. WI mice were infected with WT-HSV-2 with 10 or 10⁵ p.f.u. intranasally. Uninfected mice were used as control. Body weight gain (FIG. 17A) and mortality (FIG. 17B) of HSV-2 infected mice were monitored for to 20 days post-infection. WT mice were immunized with TK-HSV-2 (2×10⁶ p.f.u.) subcutaneously. Five to six weeks later, these mice were inoculated intranasally with 1 dose of gDTII, or RVG-OVA peptides (100μg/mouse in 10 μl, 5 μl each nostril). At day 6 post peptides administration HSV-immunized and naive mice (Non-Immunized) were challenged with a lethal dose of WT-HSV (105 p.f.u.). Five to six weeks after primary HSV-2 infection, these mice were inoculated intranasally with 1 dose of gDTII, RVG-gDTII or RVG-OVA peptides (100 μg/mouse in 10 μl, 5 μl each nostril). At day 1, 3 and 5 after peptides administration mice were injected intraperitoneally with 5 ug/500 ul/mouse of VSV mAb followed by intranasal challenge on day 7. Mortality (FIG. 17C) was measured on indicated days after challenge.

FIG. 18, comprising FIG. 18A through FIG. 18E, depicts exemplary experimental data demonstrating that intranasal antigenic peptide delivery can potentiate T cell immunotherapy for potent anti-tumor responses. FIG. 18A and FIG. 18B: mice were immunized with TK-HSV and 2 months later were implanted with GL261-Luc tumors. Mice were given OVA or gDTII peptides intranasally 6 days after tumor implantation. At day 4 and 6 after intranasal peptide stimulation, mice were treated with αPD1 or isotype antibodies and tumor growth (FIG. 18A) and survival was monitored (FIG. 18B). FIG. 18C through FIG. 18E: mice were immunized with TK-HSV, and given OVA or gDTII peptides. Four days after stimulation, mice were injected IV with fluorescent molecules to measure dye extravasation from vessels. FIG. 18D depicts the quantification of average fluorescence units of images from FIG. 18C (representative images) using IMAGEJ. FIG. 18E depicts representative vessels with similar maximum intensities that were taken to measure extravasation of fluorescent markers. Intensities were taken at a cross section from the vasculature to measure relative dye next to the vascular structure.

FIG. 19 depicts data demonstrating that intranasal delivery of MHC class II peptides opens up the BBB.

### DETAILED DESCRIPTION

The present invention provides compositions and methods of treating a disease or disorder in the central nervous system or in brain tissue. For example, in some embodiments, the invention provides compositions and methods for treating an infection of the central nervous system. In some embodiments, the invention provides compositions and methods for treating brain tumors. The present invention relates to compositions and methods for inducing a CD4 T cell response, for example a memory CD4 T cell response, in a subject to induce permeability of the blood brain barrier (BBB), allowing for a therapeutic agent to cross the BBB.

In one embodiment, the invention provides a composition for treating a disease or disorder comprising (1) an immunogenic agent (e.g., an immunogenic peptide) to induce a CD4 T cell response and (2) a therapeutic agent for the treatment of the disease or disorder. In some embodiments, the immunogenic agent is an antigenic protein or peptide. In some embodiments, the antigenic peptide is an antigen of the disease or disorder to which the therapeutic agent is directed. In some embodiments, the antigenic peptide is an antigen of a different disease or disorder than the disease or disorder to which the therapeutic agent is directed.

In one embodiment, the composition is useful for treating a pathogenic infection, where the composition comprises (1) an immunogenic agent (e.g., an antigenic peptide) to induce a CD4 T cell immune response and (2) a therapeutic agent, antibody or antibody fragment directed to an antigen of the pathogen.

In one embodiment, the composition is useful for treating cancer in the brain tissue, where the composition comprises (1) an immunogenic agent (e.g., an antigenic peptide) to induce a CD4 T cell immune response and (2) a therapeutic antibody or antibody fragment directed to an antigen associated with the cancer in the brain tissue.

In one embodiment, the invention provides a method of treating a disease or disorder in a subject comprising (1) administering to the subject an immunogenic agent to induce a CD4 T cell immune response, and (2) administering to the subject a therapeutic agent for the treatment of the disease or disorder. The method may be used to treat or prevent a disease or disorder in the brain or spinal cord. The method may be used to treat or prevent any disease or disorder of the brain or spinal cord, including, but not limited to, pathogenic infection, cancer, and neurodegenerative disease, such as Alzheimer's disease.

In one embodiment, the invention provides a method of treating a pathogenic infection in a subject comprising administering (1) an immunogenic agent (e.g., an antigenic peptide) to induce a CD4 T cell immune response and (2) a therapeutic agent, antibody or antibody fragment directed to an antigen of the pathogen. The method may be used to treat or prevent any pathogenic infection, including, but not limited to a viral infection, bacterial infection, fungal infection, parasitic infection, helminth infection, protozoan infection, prion infection and the like.

In one embodiment, the invention provides a method of treating a pathogenic infection in a subject comprising administering (1) an immunogenic agent (e.g., an antigenic peptide) to induce a CD4 T cell immune response and (2) an inhibitor of an immune checkpoint protein or pathway. In one embodiment, the checkpoint inhibitor is an antibody or antibody fragment targeted to one or more immune response checkpoint proteins. For example, in one embodiment, the second agent is an antibody or antibody fragment that specifically binds to PD-1, PDL-1 CTLA-4, LAG-3, TIM-3, CEACAM1, TIGIT or the like.

In one embodiment, the invention provides a method of treating cancer in a subject comprising administering (1) an immunogenic agent (e.g., an antigenic peptide) to induce a CD4 T cell immune response and (2) a therapeutic antibody or antibody fragment directed to an antigen associated with the tumor.

In one embodiment, the invention provides a method of treating cancer in a subject comprising administering (1) an immunogenic agent (e.g., an antigenic peptide) to induce a CD4 T cell immune response and (2) an inhibitor of an immune checkpoint protein or pathway. In one embodiment, the checkpoint inhibitor is an antibody or antibody fragment targeted to one or more immune response checkpoint proteins. For example, in one embodiment, the second agent is an antibody or antibody fragment that specifically binds to PD-1, PDL-1 CTLA-4, LAG-3, TIM-3, CEACAM1, TIGIT or the like.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20%, ±10%, ±5%, ±1%, or ±0.1% from the specified value, as such variations are appropriate to perform the disclosed methods.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody, which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned) or other technology, which is available and well known in the art.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequences or a partial nucleotide sequence encoding a protein that elicits an adaptive immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “adjuvant” as used herein is defined as any molecule to enhance an antigen-specific adaptive immune response.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) RNA, and viruses (e.g., lentiviruses, retroviruses, adenoviruses, and adeno-associated viruses) that incorporate the recombinant polynucleotide.

“Immunogen” refers to any substance introduced into the body in order to generate an immune response. That substance can a physical molecule, such as a protein, or can be encoded by a vector, such as DNA, mRNA, or a virus.

By the term “immune reaction,” as used herein, is meant the detectable result of stimulating and/or activating an immune cell.

“Immune response,” as the term is used herein, means a process that results in the activation and/or invocation of an effector function in either the T cells, B cells, natural killer (NK) cells, and/or antigen-presenting cells (APCs). Thus, an immune response, as would be understood by the skilled artisan, includes, but is not limited to, any detectable antigen-specific or allogeneic activation of a helper T cell or cytotoxic T cell response, production of antibodies, T cell-mediated activation of allergic reactions, macrophage infiltration, and the like.

“Immune cell,” as the term is used herein, means any cell involved in the mounting of an immune response. Such cells include, but are not limited to, T cells, B cells, NK cells, antigen-presenting cells (e.g., dendritic cells and macrophages), monocytes, neutrophils, eosinophils, basophils, and the like.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleosides (nucleobase bound to ribose or deoxyribose sugar via N-glycosidic linkage) are used. “A” refers to adenosine, “C” refers to cytidine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject.

The terms “patient,” “subject,” “individual,” and the like are used interchangeably herein, and refer to any animal, or cells thereof whether in vitro or in situ, amenable to the methods described herein. In some non-limiting embodiments, the patient, subject or individual is a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “therapeutic” as used herein means a treatment and/or prophylaxis. A therapeutic effect is obtained by suppression, diminution, remission, or eradication of at least one sign or symptom of a disease or disorder state.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention provides compositions and methods for treating a disease or disorder in an immunoprivilaged tissue in a subject in need thereof. The present invention is based in part upon the discovery that memory CD4 T cells are required to allow antibody access to immunoprivilaged tissue. For example, it is demonstrated herein that both antibodies and CD4 T cells are required to protect the host after immunization at a distal site. It is shown that memory CD4 T cells migrate to the immunoprivilaged tissue, secrete interferon-γ, and mediate local increase in vascular permeability, enabling antibody access. The results reveal a previously unappreciated role of CD4 T cells in mobilizing antibodies to the peripheral sites of infection where they help to limit infection.

The present invention provides a composition for treating or preventing a disease or disorder comprising a first agent and a second agent. In one embodiment, the first agent induces an immune response in the subject. For example, in one embodiment, the first agent induces the activation and production of memory CD4 T cells. In some embodiments, the first agent is an immunogenic composition (e.g., vaccine) that induces an immune response. In one embodiment, the second agent is a therapeutic agent directed to the disease or disorder. For example, in one embodiment, the second agent is an antibody or antibody fragment that specifically binds to an antigen associated with the disease or disorder. The memory CD4 T cells induced by the first agent allows the second agent to access the immunoprivilaged tissue.

The present invention provides methods for treating or preventing a disease or disorder of immunoprivilaged tissue in a subject in need thereof.

In one embodiment, the method comprises administering to the subject a first agent and a second agent. In one embodiment, the first agent induces an immune response in the subject. For example, in one embodiment, the first agent induces the activation and production of memory CD4 T cells. In some embodiments, the first agent is an immunogenic composition (e.g., vaccine) that induces an immune response. In one embodiment, the second agent is a therapeutic agent directed to the disease or disorder. For example, in one embodiment, the second agent is an antibody or antibody fragment that specifically binds to an antigen associated with the disease or disorder. In one embodiment, the method comprises administering a vaccine to induce an immune response in the subject; and administering a therapeutic antibody or antibody fragment that binds to an antigen associated with the disease or disorder.

In one embodiment, the compositions and methods of the present invention may be used to treat or prevent a disease or disorder in any immunoprivilaged tissue, including but not limited to the brain, spinal cord, peripheral nervous system, testes, eye, placenta, liver, pancreas and the like.

In one embodiment, the compositions and methods of the present invention may be used to treat or prevent any pathogenic infection, including, but not limited to a viral infection, bacterial infection, fungal infection, parasitic infection, helminth infection and the like.

In one embodiment, the compositions and methods of the present invention may be used to treat or prevent cancer.

In one embodiment, the compositions and methods of the present invention may be used to treat or prevent a neurological disorder, including, but not limited to, Alzheimer's disease.

Compositions

The present invention provides compositions for treating or preventing a disease or disorder comprising a first agent and at least one additional agent. In one embodiment, the first agent induces an immune response in the subject. In some embodiments, the first agent is an immunogenic agent (e.g., an antigenic peptide) that induces an immune response.

In one embodiment, at least one additional agent is a checkpoint inhibitor. In one embodiment, at least one additional agent is an antibody or antibody fragment targeted to one or more immune response checkpoint proteins. For example, in one embodiment, the second agent is an antibody or antibody fragment that specifically binds to PD-1, PDL-1 CTLA-4, LAG-3, TIM-3, CEACAM1, TIGIT or the like.

In one embodiment, at least one additional agent is a therapeutic agent for the treatment of the disease or disorder. In one embodiment, at least one additional agent is an antibody or antibody fragment targeted to an antigen associated with the disease or disorder. For example, in one embodiment, the second agent is an antibody or antibody fragment that specifically binds to the antigen.

Immunogenic Agent

In one embodiment, the composition of the present invention comprises an immunogenic agent. In some embodiments, the immunogenic agent comprises a peptide, nucleic acid molecule, cell, or the like, that induces an antigen-specific immune response. For example, in one embodiment, the immunogenic agent comprises an antigen. In some embodiments, the agent is associated with the disease or disorder being treated. In some embodiments, the antigen is associated with the pathogenic infection being treated. In some embodiments, the antigen is a tumor-specific antigen or a tumor-associated antigen.

In some embodiments, the immunogenic agent is a vaccine. For the immunogenic agent to be useful as a vaccine, the immunogenic agent must induce an immune response to the antigen in a cell, tissue or mammal (e.g., a human). In some embodiments, the vaccine induces a protective immune response in the mammal. In one embodiment, the vaccine induces the activation and production of memory CD4 T cells in the mammal. As used herein, an “immunogenic agent” may comprise an antigen (e.g., a peptide or polypeptide), a nucleic acid encoding an antigen (e.g., an antigen expression vector), and a cell expressing or presenting an antigen or cellular component. In some embodiments, the immunogenic agent is an inactivated pathogen, attenuated pathogen, temperature-sensitive pathogen, or the like, which can be used to induce a pathogen-specific immune response.

In some embodiments, the antigen comprises a viral antigen, including but not limited to an antigen of Influenza virua, Zika virus, Ebola virus, Japanese encephalitis virus, mumps virus, measles virus, rabies virus, varicella-zoster, Epstein-Barr virus (HHV-4), cytomegalovirus, herpes simplex virus 1 (HSV-1) and herpes simplex virus 2 (HSV-2), human immunodeficiency virus-1 (HIV-1), JC virus, arborviruses, enteroviruses, and West Nile virus, dengue virus, poliovirus, and varicella zoster virus. In some embodiments, the antigen comprises a bacterial antigen, including, but not limited to, an antigen of Streptococcus pneumoniae, Neisseria meningitides, Streptococcus agalactia, and Escherichia coli. In some embodiments, the antigen comprises a fungal or protozoan antigen, including, but not limited to, an antigen of Candidiasis, Aspergillosis, Cryptococcosis, and Toxoplasma gondii.

In some embodiments, the antigen comprises a tumor-specific antigen or a tumor-associated antigen, including but not limited to: differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA and other cancer germline associated tumor-antigens, including peptides often found in intronic regions or non-coding regions such as NY-ESO1, MAGE-C family and antigens derived from endogenous retro-elements, overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens, influenza viral antigens, CMV viral antigens, vesicular stomatitis virus (VSV) antigens and human papillomavirus (HPV) antigens. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, Aim2, Art-4, EphA2, EZH2, Fosl1, PTH-rP, Sox 11, Whsc2, YKL-40 and TPS.

In some embodiments, the immunogenic agent is a MHC class II antigenic peptide. MHC class II peptides are antigenic peptides that are loaded on to MHC class II molecules, and the entire complex migrates to the cell membrane surface, where peptide specific CD4 T cells (helper T cells) can recognize it. MHC class II molecules include, but are not limited to HLA-DP, HLA-DM, HLA-DOA, HLA-DOB, HLA-DQ, HLA-DR, HLA-DPA1, HLA-DPB1, HLA-DQA1, HLA-DQB1, HLA-DRA, HLA-DRB1 and subsets including -A1 -B1 to -B3 to -B5.

Exemplary antigenic peptides include, but are not limited to:

| | | | |
|---|---|---|---|
| | | SEQ | class II |
| | | ID | MHC |
| Protein | Peptide | NO: | molecule |

EBV

| | | | |
|---|---|---|---|
| BHRF1 | TVVLRYHLLEEY | 5 | HLADR4 |
| BALF1 | AGLTLSLLVICSYLFISR | 6 | HLADR2 |
| BYRF1 | TVFYNIPPMPL | 7 | HLA |
| | | | DQ2/DQ7 |
| EBNA1 | TSLYNLRRGTALA | 8 | HLA DR1 |
| | LDLDFGQLTPHTKAV | 9 | |
| BZLF1 | VKFTPDPYQVPFVQA | 10 | DRB3*01 |
| (11-25) | | | |
| BZLF1 | LTAYHVSTAPTGSWF | 11 | DRB3*01 |
| (61-75) | | | |
| BZLF1 | PGDNSTVQTAAAVVF | 12 | DRB1*13 |
| (116-130) | | | |
| BZLF1 | PPVKRKKGLRDSREG | 13 | DRB1*08 |
| (407-421) | | | |
| BMLF1 | DEDPTPAHAIPARPS | 14 | DQB1*07 |
| (41-55) | | | |
| BMHRF1 | PYYVVDLSVRGM | 15 | DRB1*04 |
| (122-133) | | | |
| BMRF1 | VKLTMEYDDKVSKSH | 16 | DRBl*0301 |
| (136-150) | | | |
| BKRF2 | GSFSVEDLFGANLNRYAWHR | 17 | DPB1*04 |
| (116-135) | | | |
| BKZLF2 | QIFGSHCTYVSKFSTVPVSH | 18 | DRB1*16 |
| (186-205) | | | |
| BLLF1 | LDLDFGQLTPHTKAVYQPRG | 19 | DRB1*15 |
| (61-81) | | | |
| BALF4 | DNEIFLTKKMTEVCQ | 20 | DRB1*08 |
| (575-589) | | | |
| BXLF2 | LEKQLFYYIGTMLPNTRPHS | 21 | DRB5*01 |
| (126-140) | | | |

FLU VIRUS

| | | | |
|---|---|---|---|
| | PKYVKQNTLKLAT | 22 | HLA-DRA*01 |
| NP | VFELSDEKAASPI | 23 | DRB1*0901 |
| NP | GQISIQPTFSVQR | 24 | DRB1*0404 |
| M1 | RQMVQAMRTIG | 25 | DPB1*0301 |
| M1 | GLIYNRMGAVTTE | 26 | DRB1*0101 |
| M1(43-55) | MEWLKTRPILSPL | 27 | DPB1*0401 |
| M1(94-106) | DKAVKLYRKLKRE | 28 | DRB1*1301 |
| M1(101-113) | RKLKREITFHGAKK | 29 | DRB1*0701 |
| M1(105-117) | GLIYNRMGAVTTE | 30 | DRB1*0701 |
| M1(209-221) | ARQMVQAMRTIGTT | 31 | DRB1*0404 |
| NP(102-114) | GKWMRELILYDKER | 32 | |
| NP(115-127) | EIRRIWRQANNGD | 33 | DRB1*0301 |
| NP(404-416) | GQISIQPTFSVQR | 34 | DRB1*1401 |
| NP(463-475) | VFELSDEKAASPI | 35 | |
| H2 HA339- | SRGLFGAIAGFIEGGWQ | 36 | DRB1*01:09 |
| 355 | | | and *01:01 |
| H1 HA340- | IQSRGLFGAIAGFIEGG | 37 | |
| 356 | | | |
| H3 HA338- | NVPEKQTRGIFGAIAGF | 38 | |
| 354 | | | |
| H3 HA344- | TRGIFGAIAGFIENGWE | 39 | |
| 360 | | | |
| H5 HA345- | KRGLFGAIAGFIEGGWQ | 40 | |
| 361 | | | |
| B/HA360-376 | ERGFFGAIAGFLEGGWE | 41 | |
| NS1-11 | ESDEAFKMTMASALASR | 42 | DR1-Tg |
| | | | mice from |
| | | | Merk |
| NS1-21 | LTLLRAFTEEGAIVGEI | 43 | DR1-Tg |
| | | | mice from |
| | | | Merk |
| NS1-22 | FTEEGAIVGEISPLPSL | 44 | DR1-Tg |
| | | | mice from |
| | | | Merk |
| NPp74 | SDMRAEIIKMMESARPE | 45 | DR1-Tg |
| | | | mice from |
| | | | Merk |
| NPp75 | IIKMMESARPEEVSFQG | 46 | DR1-Tg |
| | | | mice from |
| | | | Merk |
| HA33-52 | VDTVLEKNVTVTHSVNLLED | 47 | DR*07:01 |
| HA113-132 | IDYEELREQLSSVSSFERFE | 48 | DRB5 |
| HA121-140 | QLSSVSSFERFEIFPKTSSW | 49 | DR1101 |
| | | | and DRB5 |
| HA161-180 | FYKNLIWLVKKGNSYPKLSK | 50 | DR1101 |
| | | | and DRB5 |
| HA249-268 | TLVEPGDKITFEATGNLVVP | 51 | DR07:01 |
| HA257-276 | ITFEATGNLVVPRYAFAMER | 52 | DR07:01 |
| HA265-284 | LVVPRYAFAMERNAGSGIII | 53 | DR04:01 |
| HA305-324 | TSLPFQNIHPITIGKCPKYV | 54 | DR0701 |
| HA307-319 | PKYVKQNTLKLAT | 55 | DRB*04:01 |

CMV

| | | | |
|---|---|---|---|
| pp65 108-127 | MSIYVYALPLKMLNIPSINVH | 56 | DR0101 |
| pp65 115-127 | LPLKMLNIPSINVH | 57 | DR0101 |
| pp65 14-25 | VLGPISGHVLKA | 58 | |
| pp65 47-57 | HVRVSQPSLIL | 59 | |
| pp65 337-349 | VELRQYDPVAALF | 60 | |
| pp65 370-389 | IVKPGDILVTNSNGNLIA | 61 | |
| pp65 494-511 | RNLVPMVATVQGQNLKYQ | 62 | |
| pp65 518-531 | NDIYRIFAELEGVW | 63 | |
| | LLQTGIHVRVSQPSL | 64 | DR15 (DQ6) |
| | IIKPGKISHIMLDVA | 65 | DR53 (DQ3) |
| | PQYSEHPTFTSQYRI | 66 | DR11 |
| | EHPTFTSQYRIQGKL | 67 | |
| | FTSQYRIQGKLEYRH | 68 | |
| | QEFFWDANDIYRIFA | 69 | DR52 |
| | FFWDANDIYRIFAEL | 70 | |
| | AGILARNLVPMVATV | 71 | |
| | PPWQAGILARNLVPMV | 72 | |
| | ARNLVPMVATVQGQN | 73 | |
| | LLQTGIHVRVSQPSL | 74 | DR15 |
| | PLKMLNIPSINVHHY | 75 | DR1/DR3 |
| | TRQQNQWKEPDVYYT | 76 | DR1 |
| | EPDVYYTSAFVFPTK | 77 | DR7 |
| | KVYLESFCEDVPSGK | 78 | DR15 |
| | TLGSDVEEDLTMTRN | 79 | DR3 |
| | QPFMRPHERNGFTVL | 80 | DR13 |
| | IIKPGKISHIMLDVA | 81 | DR4/DR7 |
| | EHPTFTSQYRIQGKL | 82 | DR11 |
| | YRIQGKLEYRHTWDR | 83 | DR3 |
| | TERKTPRVTGGGAMA | 84 | DR14 |
| | ASTSAGRKRKSASSA | 85 | DR11 |
| | ACTSGVMTRGRLKAE | 86 | DR1 |
| | AGILARNLVPMVATV | 87 | DR11 DR3 |
| | KYQEFFWDANDIYRI | 88 | DR1 DR3 |

HSV

| | | |
|---|---|---|
| RVG-gDT | YTIWMPENPRPGTPCDIFTNSR | 89 |
| | GKRASNGGGGCCIPPNWHIPSI | |
| | QDA | |
| gD315-327 | IPPNWHIPSIQDA | 90 |

In some embodiments, the antigen comprises an antigen associated with a neurological disorder. Exemplary antigens associated with a neurological disorder include, but are not limited to various monomeric and aggregated forms of A3, tau, BACE1, α-synuclein, huntingtin, TAR-DNA binding protein 43 kDA, superoxide dismutase 1, prion protein, and fragments thereof.

In some embodiments, the immunogenic agent comprises a full length protein associated with a pathogen, a tumor or a neurological disorder. For example, in one embodiment, the immunogenic agent comprises full length tetanus toxoid (TT) protein.

The antigenic peptide or protein of the present invention may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The invention should also be construed to include any form of a protein or peptide having substantial homology to a protein or peptide disclosed herein. In some embodiments, a peptide which is “substantially homologous” is about 50% homologous, about 70% homologous, about 80% homologous, about 90% homologous, about 95% homologous, or about 99% homologous to amino acid sequence a parental protein or peptide.

The antigenic peptide or protein may alternatively be made by recombinant means or by cleavage from a longer polypeptide. The composition of a peptide may be confirmed by amino acid analysis or sequencing.

The variants of the antigenic peptides or proteins according to the present invention may be (i) one in which one or more of the amino acid residues are substituted with a conserved or non-conserved amino acid residue and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one polypeptide to a sequence of a second polypeptide. Variants are defined to include peptide sequences different from the original sequence, for example, different from the original sequence in less than 40% of residues per segment of interest, in less than 25% of residues per segment of interest, in less than 10% of residues per segment of interest, or in just a few residues per segment of interest and at the same time sufficiently homologous to the original sequence to preserve the functionality of the original sequence. The present invention includes amino acid sequences that are at least 60%, 65%, 70%, 72%, 74%, 76%, 78%, 80%, 90%, 91%, 92%, 93%, 94%, 95%, 96%, 97%, 98%, or 99% similar or identical to the original amino acid sequence. The degree of identity between two peptides is determined using computer algorithms and methods that are widely known for the persons skilled in the art. In some embodiments, the identity between two amino acid sequences is determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The antigenic peptides or proteins of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction. An antigenic peptide or protein of the invention may be phosphorylated using conventional methods such as the method described in Reedijk et al. (The EMBO Journal 11(4):1365, 1992).

The antigenic peptides or proteins of the invention may include unnatural amino acids formed by post-translational modification or by introducing unnatural amino acids during translation. A variety of approaches are available for introducing unnatural amino acids during protein translation.

An antigenic peptide or protein of the invention may be conjugated with other molecules, such as proteins, to prepare fusion proteins. This may be accomplished, for example, by the synthesis of N-terminal or C-terminal fusion proteins provided that the resulting fusion protein retains the functionality of inducing a CD4 T cell immune response.

Cyclic derivatives of the peptides of the invention are also part of the present invention. Cyclization may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulfide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The invention also relates to antigenic peptides fused to, or integrated into, a target protein, and/or a targeting domain capable of directing the chimeric protein to a desired cellular component or cell type or tissue. The chimeric proteins may also contain additional amino acid sequences or domains. The chimeric proteins are recombinant in the sense that the various components are from different sources, and as such are not found together in nature (i.e., are heterologous).

In one embodiment, the targeting domain can be a membrane spanning domain, a membrane binding domain, or a sequence directing the protein to associate with for example vesicles or with the nucleus. In one embodiment, the targeting domain can target a peptide to a particular cell type or tissue. For example, the targeting domain can be a cell surface ligand or an antibody against cell surface antigens of a target tissue. A targeting domain may target the peptide of the invention to a cellular component.

An antigenic peptide of the invention may be synthesized by conventional techniques. For example, the peptides or chimeric proteins may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, Vol 1, for classical solution synthesis). By way of example, a peptide of the invention may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a peptide or chimeric protein of the invention conjugated with other molecules may be prepared by fusing, through recombinant techniques, the N-terminal or C-terminal of the peptide or chimeric protein, and the sequence of a selected protein or selectable marker with a desired biological function. The resultant fusion proteins contain the antigenic peptide or protein fused to the selected protein or marker protein as described herein. Examples of proteins which may be used to prepare fusion proteins include immunoglobulins, glutathione-S-transferase (GST), hemagglutinin (HA), and truncated myc.

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The peptides and chimeric proteins of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

In one embodiment, the present invention provides a composition comprising an isolated nucleic acid encoding an antigenic peptide or protein, or a biologically functional fragment thereof.

The isolated nucleic acid sequence encoding the antigenic protein or peptide can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The isolated nucleic acid may comprise any type of nucleic acid, including, but not limited to DNA and RNA. For example, in one embodiment, the composition comprises an isolated DNA molecule, including for example, an isolated cDNA molecule, encoding the antigenic protein or peptide, or functional fragment thereof. In one embodiment, the composition comprises an isolated RNA molecule encoding the antigenic protein or peptide, or a functional fragment thereof.

The nucleic acid molecules of the present invention can be modified to improve stability in serum or in growth medium for cell cultures. Modifications can be added to enhance stability, functionality, and/or specificity and to minimize immunostimulatory properties of the nucleic acid molecule of the invention. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule.

In one embodiment of the present invention the nucleic acid molecule may contain at least one modified nucleotide analogue. For example, the ends may be stabilized by incorporating modified nucleotide analogues.

Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In some backbone-modified ribonucleotides the phosphoester group connecting to adjacent ribonucleotides is replaced by a modified group, e.g., of phosphothioate group. In some sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In some embodiments, the nucleic acid molecule comprises at least one of the following chemical modifications: 2′-H, 2′-O-methyl, or 2′-OH modification of one or more nucleotides. In certain embodiments, a nucleic acid molecule of the invention can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target.

In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

Nucleic acid agents discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, for example, as occur naturally in the human body. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, for example, different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone.

Modifications of the nucleic acid of the invention may be present at one or more of, a phosphate group, a sugar group, backbone, N-terminus, C-terminus, or nucleobase.

The present invention also includes a vector in which the isolated nucleic acid of the present invention is inserted. The art is replete with suitable vectors that are useful in the present invention.

In some embodiments, the expression of natural or synthetic nucleic acids encoding an antigenic protein or peptide is typically achieved by operably linking a nucleic acid encoding the antigenic protein or peptide or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method

In certain embodiments, the vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In one embodiment, the vector of the present invention comprises one or more enhancers to boost transcription of the gene present within the vector.

In order to assess the expression of an antigenic protein or peptide, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). In one embodiment, the method of introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In one embodiment, the present invention provides a delivery vehicle comprising an antigenic protein or peptide, or a nucleic acid molecule encoding an antigenic protein or peptide. Exemplary delivery vehicles include, but are not limited to, microspheres, microparticles, nanoparticles, polymerosomes, liposomes, and micelles. For example, in certain embodiments, the delivery vehicle is loaded with an antigenic protein or peptide, or a nucleic acid molecule encoding an antigenic protein or peptide. In certain embodiments, the delivery vehicle provides for controlled release, delayed release, or continual release of its loaded cargo. In certain embodiments, the delivery vehicle comprises a targeting moiety that targets the delivery vehicle to a treatment site.

In particular embodiments the immunogenic agent comprises or encodes all or part of any antigen described herein, or an immunologically functional equivalent thereof. In other embodiments, the immunogenic agent is in a mixture that comprises an additional immunostimulatory agent or nucleic acids encoding such an agent. Immunostimulatory agents include but are not limited to an additional antigen, an immunomodulator, an antigen presenting cell or an adjuvant. In other embodiments, one or more of the additional agent(s) is covalently bonded to the antigen or an immunostimulatory agent, in any combination. In some embodiments, the immunogenic agent is conjugated to or comprises an HLA anchor motif amino acids. In some instances, the immunogenic agent of the invention can be used to induce an antigen-specific immune response, including the production of memory CD4 T cells, in the subject.

A vaccine of the present invention may vary in its composition of peptides, nucleic acids and/or cellular components. In a non-limiting example, an antigen might also be formulated with an adjuvant. Of course, it will be understood that various compositions described herein may further comprise additional components. For example, one or more vaccine components may be comprised in a lipid or liposome. In another non-limiting example, a vaccine may comprise one or more adjuvants. A vaccine of the present invention, and its various components, may be prepared and/or administered by any method disclosed herein or as would be known to one of ordinary skill in the art, in light of the present disclosure.

Exemplary adjuvants include, but is not limited to, PAMPs, DAMPs, alpha-interferon, gamma-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. Other genes which may be useful adjuvants include those encoding: MCP-I, MIP-Ia, MIP-Ip, IL-8, RANTES, L-selectin, P-selectin, E-selectin, CD34, GlyCAM-1, MadCAM-1, LFA-I, VLA-I, Mac-1, p150.95, PECAM, ICAM-I, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-I, DR3, TRAMP, Apo-3, AIR, LARD, NGRF, DR4, DR5, KILLER, TRAIL-R2, TRICK2, DR6, Caspase ICE, Fos, c-jun, Sp-I, Ap-I, Ap-2, p38, p65Rel, MyD88, IRAK, TRAF6, IkB, Inactive NIK, SAP K, SAP-I, INK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP 1, TAP2, anti-CTLA4-sc, anti-LAG3-Ig, anti-TIM3-Ig and functional fragments thereof.

In one embodiment, the peptide vaccine of the invention includes, but is not limited to a peptide mixed with adjuvant substances and a peptide which is introduced together with an APC. The most common cells used for the latter type of vaccine are bone marrow and peripheral blood derived dendritic cells, as these cells express costimulatory molecules that help activation of T cells. WO00/06723 discloses a cellular vaccine composition which includes an APC presenting tumor associated antigen peptides. Presenting the peptide can be effected by loading the APC with a polynucleotide (e.g., DNA, RNA, etc.) encoding the peptide or loading the APC with the peptide itself.

When an immunogenic agent induces an anti-pathogen immune response upon inoculation into an animal, the immunogenic agent is decided to have anti-pathogen immunity inducing effect. The pathogen-specific immune response can be detected by observing in vivo or in vitro the response of the immune system in the host against the peptide.

For example, a method for detecting the induction of cytotoxic T lymphocytes is well known. A foreign substance that enters the living body is presented to T cells and B cells by the action of APCs. T cells that respond to the antigen presented by APC in an antigen specific manner differentiate into cytotoxic T cells (also referred to as cytotoxic T lymphocytes or CTLs) due to stimulation by the antigen. These antigen stimulated cells then proliferate. This process is referred to herein as “activation” of T cells. Therefore, CTL induction by a certain peptide or combination of peptides of the invention can be evaluated by presenting the peptide to a T cell by APC, and detecting the induction of CTL. Furthermore, APCs have the effect of activating CD4⁺ T cells, CD8⁺ T cells, macrophages, eosinophils and NK cells.

A method for evaluating the inducing action of CTL using dendritic cells (DCs) as APC is well known in the art. DC is a representative APC having the strongest CTL inducing action among APCs. In this method, the peptide or combination of peptides are initially contacted with DC and then this DC is contacted with T cells. Detection of T cells having cytotoxic effects against the cells of interest after the contact with DC shows that the peptide or combination of peptides have an activity of inducing the cytotoxic T cells. Furthermore, the induced immune response can be also examined by measuring IFN-gamma produced and released by CTL in the presence of antigen-presenting cells that carry immobilized peptide or combination of peptides by visualizing using anti-IFN-gamma antibodies, such as an ELISPOT assay.

Apart from DC, peripheral blood mononuclear cells (PBMCs) may also be used as the APC. The induction of CTL is reported to be enhanced by culturing PBMC in the presence of GM-CSF and IL-4. Similarly, CTL has been shown to be induced by culturing PBMC in the presence of keyhole limpet hemocyanin (KLH) and IL-7.

The induction of a pathogen-specific immune response can be further confirmed by observing the induction of antibody production against the specific pathogen. In one embodiment, the induction of a pathogen-specific immune response can be further confirmed by observing the activation and production of memory CD4 T cells.

Therapeutic Agent

In one embodiment, the composition comprises a therapeutic agent. In some embodiments, the therapeutic agent comprises a peptide, nucleic acid molecule, small molecule, antibody, or the like. In some embodiments, the therapeutic agent is for the treatment of a disease or infection of the brain or spinal cord. For example, in some embodiments, the therapeutic agent comprises an antibody or antibody fragment that binds to a pathogen or antigen of a pathogen. In some embodiments, the therapeutic agent comprises an antibody or antibody fragment that binds to a tumor-specific antigen or tumor-associated antigen. In some embodiments, the therapeutic agent comprises an antibody or antibody fragment that binds to an antigen associated with a neurological disease.

In one embodiment, the therapeutic agent comprises a checkpoint inhibitor. In some embodiments, the combination of antigen and immune checkpoint antibody induces the immune system more efficiently than an immunogenic composition comprising the antigen alone. This more efficient immune response provides increased efficacy in the treatment and/or prevention of any disease, in particular cancer, or one caused by a pathogen, such as a virus. In one embodiment, the checkpoint inhibitor inhibits at least one of PD-1, PDL-1 CTLA-4, LAG-3, TIM-3, TIGIT and CEACAM1. Exemplary checkpoint inhibitors that can be used in the compositions and methods of the invention include, but are not limited to, ipilimumab, nivolumab, pembrolizumab, pidilizumab, atezolizumab, BMS-986016, BMS-936559, MPDL3280A, MDX1105-01, MEDI4736, TSR-022, CM-24 and MK-3475.

In one embodiment, the therapeutic agent comprises a therapeutic antibody or antibody fragment. The therapeutic antibody or antibody fragment includes any antibody known in the art which binds a pathogen, induces the killing of a pathogen, reduces pathogenic infection, or prevents spread of a pathogenic infection. The therapeutic antibody or antibody fragment includes any antibody known in the art which binds to a tumor cell, induces the killing of the tumor cell, or prevents tumor cell proliferation or metastasis. In some embodiments, the therapeutic agent comprises a T-cell that has been modified to express an antibody or antibody fragment (e.g., chimeric antigen receptor T-cell, Bi-specific T-cell engaging antibodies and other forms). In one embodiment, the therapeutic agent comprises an antibody-drug conjugate.

In some embodiments, the therapeutic antibody or antibody fragment binds to the same antigen of the immunogenic agent. In some embodiments, the antigen to which therapeutic antibody or antibody fragment binds to a different from the antigen of the immunogenic agent. In some embodiments, the antigen to which the therapeutic agent binds and the antigen of the immunogenic agent are each associated with the same disease, disorder, or infection.

Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, bind to the specific antigens of interest, and they are able to bind the antigen present on Western blots, in solution in enzyme linked immunoassays, in fluorescence activated cells sorting (FACS) assays, in magenetic-actived cell sorting (MACS) assays, and in immunofluorescence microscopy of a cell transiently transfected with a nucleic acid encoding at least a portion of the antigenic protein, for example.

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest. When the antibody used in the invention is humanized, the antibody may be generated as described in Queen, et al. (U.S. Pat. No. 6,180,370), Wright et al., (supra) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as an epitope on an antigen of interest, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

The invention also includes functional equivalents of the antibodies described herein. Functional equivalents have binding characteristics comparable to those of the antibodies, and include, for example, hybridized and single chain antibodies, as well as fragments thereof. Methods of producing such functional equivalents are disclosed in PCT Application WO 93/21319 and PCT Application WO 89/09622.

Functional equivalents include polypeptides with amino acid sequences substantially the same as the amino acid sequence of the variable or hypervariable regions of the antibodies. “Substantially the same” amino acid sequence is defined herein as a sequence with at least 70%, at least about 80%, at least about 90%, at least about 95%, or at least 99% homology to another amino acid sequence (or any integer in between 70 and 99), as determined by the FASTA search method in accordance with Pearson and Lipman, 1988 Proc. Nat'l. Acad. Sci. USA 85: 2444-2448. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′)₂ fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H2L2) formed of two dimers associated through at least one disulfide bridge.

Methods

The invention provides a method for treating, or preventing infection or a disease or disorder of the brain, central nervous system or spinal cord. The therapeutic compounds or compositions of the invention may be administered prophylactically or therapeutically to subjects suffering from or at risk of (or susceptible to) developing the disease or disorder. Such subjects may be identified using standard clinical methods. In the context of the present invention, prophylactic administration occurs prior to the manifestation of overt clinical symptoms, such that an infection is prevented or alternatively delayed in its progression. In the context of the field of medicine, the term “prevent” encompasses any activity which reduces the burden of mortality or morbidity from the disease or disorder. Prevention can occur at primary, secondary and tertiary prevention levels. While primary prevention avoids the development of a disease, secondary and tertiary levels of prevention encompass activities aimed at preventing the progression of an infection and the emergence of symptoms as well as reducing the negative impact of an already established disease by restoring function and reducing disease or disorder-related complications.

In one embodiment, the method comprises administering to the subject a composition comprising an immunogenic agent (e.g., an antigenic protein or peptide), as described elsewhere herein. In one embodiment, the composition comprises an adjuvant. An adjuvant refers to a compound that enhances the immune response against the peptide or combination of peptides when administered together (or successively) with the peptide having immunological activity. Examples of suitable adjuvants include cholera toxin, salmonella toxin, alum and such, but are not limited thereto. Furthermore, a vaccine of this invention may be combined appropriately with a pharmaceutically acceptable carrier. Examples of such carriers are sterilized water, physiological saline, phosphate buffer, culture fluid and such. Furthermore, the vaccine may contain as necessary, stabilizers, suspensions, preservatives, surfactants and such. The vaccine is administered systemically or locally. Vaccine administration may be performed by single administration or boosted by multiple administrations.

In one embodiment, the antigenic proteins or peptides of the invention are used in an ex vivo method to generate cells of the invention (e.g., peptide-load antigen presenting cells or peptide-specific IFNγ-secreting CD4+ T cells). In one embodiment, the disease or disorder may be treated or prevent, for example, by administering the cells of the invention. For example, PBMCs of the subject receiving treatment or prevention are collected, contacted ex vivo with an antigen or nucleic acid encoding an antigen. Following the induction of peptide-load antigen presenting cells or peptide-specific IFNγ-secreting CD4+ T cells, the cells may be administered to the subject. The cells can be induced by introducing a vector encoding the peptide or combination of peptides into them ex vivo. The cells induced in vitro can be cloned prior to administration. By cloning and growing cells having high activity of damaging target cells, cellular immunotherapy can be performed more effectively. Furthermore, cells of the invention isolated in this manner may be used for cellular immunotherapy not only against individuals from whom the cells are derived, but also against similar types of diseases in other individuals.

In one embodiment, the method comprises administering to the subject an immune checkpoint inhibitor, as described elsewhere herein. For example, in one embodiment, the method comprises administering an antibody or antibody fragment that binds to an immune checkpoint protein.

In one embodiment, the method comprises administering to the subject a therapeutic agent, as described elsewhere herein. For example, in one embodiment, the method comprises administering a therapeutic antibody or antibody fragment that binds to an antigen.

The different agents may be administered to the subject in any order and in any suitable interval. For example, in some embodiments, two or more of the immunogenic agent, the immune checkpoint inhibitor and the therapeutic agent are administered simultaneously or near simultaneously. In some embodiments, the method comprises a staggered administration of the agents, where the immunogenic agent is administered and at least one of the immune checkpoint inhibitor and the therapeutic agent are administered at some later time point. In some embodiments, the method comprises a staggered administration of the agents, where at least one of the immune checkpoint inhibitor and the therapeutic agent is administered and the immunogenic agent is administered at some later time point. Any suitable interval of administration which produces the desired therapeutic effect may be used.

The method of the present invention may be used to treat any pathogenic infection of the brain, CNS or spinal cord. For example, the method may be used to treat or prevent infections caused by a virus, a fungus, a protozoan, a parasite, an arthropod, a prion, a mycobacterium, or a bacterium, including a bacterium that has developed resistance to one or more antibiotics. Exemplary viral infections treated or prevented by way of the present method include, but is not limited to infections caused by Zika virus, ebola virus, Japanese encephalitis virus, mumps virus, measles virus, rabies virus, varicella-zoster, Epstein-Barr virus (HHV-4), cytomegalovirus, herpes simplex virus 1 (HSV-1) and herpes simplex virus 2 (HSV-2), human immunodeficiency virus-1 (HIV-1), JC virus, arborviruses, enteroviruses, and West Nile virus, dengue virus, poliovirus, and varicella zoster virus. Exemplary bacterial infections treated or prevented by way of the present method include, but is not limited to infections caused by Streptococcus pneumoniae, Neisseria meningitides, Streptococcus agalactia, and Escherichia coli. Exemplary fungal or protozoan infections treated or prevented by way of the present method include, but is not limited to infections caused by Candidiasis, Aspergillosis, Cryptococcosis, and Toxoplasma gondii.

In some embodiments, the present invention provides a method for treating or preventing a disease or disorder associated with infection of the brain, CNS or spinal cord, including but not limited to meningitis, encephalitis, meningoencephalitis, epidural abscess, subdural abscess, brain abscess, and progressive multifocal leukoencephalopathy (PML).

The method of the present invention may be used to treat or prevent cancer. The method may be used to reduce tumor growth, proliferation, or metastasis in the brain, CNS or spinal cord. Exemplary forms of cancer treated or prevented by way of the present invention, include, but are not limited to glioblastoma, meningioma, acoustic neuroma, astrocytoma, chordoma, CNS lymphoma, craniopharyngioma, brain stem glioma, ependymoma, mixed glioma, optic nerve glioma, supependymoma, medullablastoma, meningioma, metastatic brain tumors, oligodendroglioma, pituitary tumors, primitive neuroectodermal, schwannoma, juvenile pilocytic astrocytoma, pineal tumor, rhaboid tumor, spinal cancer, spinal cord tumors and pediatric brain tumors.

The method of the present invention may be used to treat or prevent a neurological disorder. Exemplary neurological disorders treated or prevented by way of the present invention, include, but are not limited to Alzheimer's disease, Parkinson's disease, tauopathy, frontotemporal dementia, Huntington's disease, prion disease, and genetic diseases of the CNS including, but not limited to, Hurler's syndrome.

The treatment and prophylactic methods of the invention may be used to treat or prevent a disease or disorder of the brain, CNS or spinal cord in any subject in need. For example, in some embodiments, the subject includes, but is not limited to humans and other primates and mammals including commercially relevant mammals such as non-human primates, cattle, pigs, horses, sheep, cats, dogs, rats, and mice.

In one embodiment, the invention provides a method of treating a disease or disorder in a subject comprising (1) administering to the subject an immunogenic agent (e.g., an antigenic peptide) to induce a CD4 T cell immune response, and (2) administering to the subject a therapeutic agent for the treatment of the disease or disorder. The method may be used to treat or prevent a disease or disorder in the brain or spinal cord. The method may be used to treat or prevent any disease or disorder of the brain or spinal cord, including, but not limited to, pathogenic infection, cancer, and neurodegenerative disease, such as Alzheimer's disease.

In one embodiment, the invention provides a method of treating a pathogenic infection in a subject comprising administering (1) an immunogenic agent (e.g., an antigenic peptide) to induce a CD4 T cell immune response and (2) a therapeutic agent, antibody or antibody fragment directed to an antigen of the pathogen. The method may be used to treat or prevent any pathogenic infection, including, but not limited to a viral infection, bacterial infection, fungal infection, parasitic infection, helminth infection, protozoan infection, prion infection and the like.

In one embodiment, the invention provides a method of treating a pathogenic infection in a subject comprising administering (1) an immunogenic agent (e.g., an antigenic peptide) to induce a CD4 T cell immune response and (2) an inhibitor of an immune checkpoint protein or pathway. In one embodiment, the checkpoint inhibitor is an antibody or antibody fragment targeted to one or more immune response checkpoint proteins. For example, in one embodiment, the second agent is an antibody or antibody fragment that specifically binds to PD-1, PDL-1 CTLA-4, LAG-3, TIM-3, CEACAM1, TIGIT or the like.

In one embodiment, the invention provides a method of treating cancer in a subject comprising administering (1) an immunogenic agent (e.g., an antigenic peptide) to induce a CD4 T cell immune response and (2) a therapeutic antibody or antibody fragment directed to an antigen associated with the tumor.

In one embodiment, the invention provides a method of treating cancer in a subject comprising administering (1) an immunogenic agent (e.g., an antigenic peptide) to induce a CD4 T cell immune response and (2) an inhibitor of an immune checkpoint protein or pathway. In one embodiment, the checkpoint inhibitor is an antibody or antibody fragment targeted to one or more immune response checkpoint proteins. For example, in one embodiment, the second agent is an antibody or antibody fragment that specifically binds to PD-1, PDL-1 CTLA-4, LAG-3, TIM-3, CEACAM1, TIGIT or the like.

In one aspect, the present invention provides a method comprising administering one or more compositions or agents, as described herein, to a subject having a disease or disorder. For example, in one embodiment, the method comprises administering one or more compositions or agents, as described herein, to a subject having a disease or disorder in the brain or spinal cord.

In one embodiment, the subject has a pathogenic infection, such as a viral infection, bacterial infection, fungal infection, parasitic infection, helminth infection, protozoan infection, prion infection and the like. In one embodiment, the method comprises administering to the subject (1) an immunogenic agent (e.g., an antigenic peptide) to induce a CD4 T cell immune response and (2) a therapeutic agent, antibody or antibody fragment directed to an antigen of the pathogen. In one embodiment, the method comprises administering (1) an immunogenic agent (e.g., an antigenic peptide) to induce a CD4 T cell immune response and (2) an inhibitor of an immune checkpoint protein or pathway. In one embodiment, the immunogenic agent comprises an antigenic peptide comprising the amino acid sequence of one of SEQ ID NOs: 5-90. In one embodiment, the checkpoint inhibitor is an antibody or antibody fragment targeted to one or more immune response checkpoint proteins. For example, in one embodiment, the second agent is an antibody or antibody fragment that specifically binds to PD-1, PDL-1 CTLA-4, LAG-3, TIM-3, CEACAM1, TIGIT or the like.

In one embodiment, the subject has a neurological disorder, such as Alzheimer's disease, Parkinson's disease, tauopathy, frontotemporal dementia, Huntington's disease, prion disease, and genetic diseases of the CNS including, but not limited to, Hurler's syndrome. In one embodiment, the method comprises (1) administering to the subject an immunogenic agent (e.g., an antigenic peptide) to induce a CD4 T cell immune response, and (2) administering to the subject a therapeutic agent for the treatment of the disease or disorder. In one embodiment, the immunogenic agent comprises an antigenic peptide comprising the amino acid sequence of one of SEQ ID NOs: 5-90.

In one embodiment, the subject has cancer or a cancerous tumor, including but not limited to glioblastoma, meningioma, acoustic neuroma, astrocytoma, chordoma, CNS lymphoma, craniopharyngioma, brain stem glioma, ependymoma, mixed glioma, optic nerve glioma, supependymoma, medullablastoma, meningioma, metastatic brain tumors, oligodendroglioma, pituitary tumors, primitive neuroectodermal, schwannoma, juvenile pilocytic astrocytoma, pineal tumor, rhaboid tumor, spinal cancer, spinal cord tumors and pediatric brain tumors. In one embodiment, the method comprises administering to the subject (1) an immunogenic agent (e.g., an antigenic peptide) to induce a CD4 T cell immune response and (2) a therapeutic agent, antibody or antibody fragment directed to an antigen associated with the tumor. In one embodiment, the method comprises administering (1) an immunogenic agent (e.g., an antigenic peptide) to induce a CD4 T cell immune response and (2) an inhibitor of an immune checkpoint protein or pathway. In one embodiment, the immunogenic agent comprises an antigenic peptide comprising the amino acid sequence of one of SEQ ID NOs: 5-90. In one embodiment, the checkpoint inhibitor is an antibody or antibody fragment targeted to one or more immune response checkpoint proteins. For example, in one embodiment, the second agent is an antibody or antibody fragment that specifically binds to PD-1, PDL-1 CTLA-4, LAG-3, TIM-3, CEACAM1, TIGIT or the like.

In some embodiments, the method comprises further administering an additional therapeutic agent, including, but not limited to, an antibiotic, antiviral agent, antifungal agent, and anti-inflammatory agent. In one embodiment, the antibiotic is selected from Amoxicillin, Ampicillin, Cloxacillin, Dicloxacillin, Nafcillin, Oxacillin, Penicillin G, Penicillin V, Piperacillin, Cefadroxil (cefadroxyl), Cefalexin (cephalexin), Cefalotin (cephalothin), Cefapirin (cephapirin), Cefazolin (cephazolin), Cefradine (cephradine), Cefaclor, Cefotetan, Cefoxitin, Cefprozil (cefproxil), Cefuroxime, Cefdinir, Cefixime, Cefotaxime, Cefpodoxime, Ceftizoxime, Ceftriaxone, Ceftazidime, Cefepime, Ceftobiprole, Ceftaroline, Aztreonam, Imipenem, Imipenem, cilastatin, Doripenem, Meropenem, Ertapenem, Azithromycin, Erythromycin, Clarithromycin, Dirithromycin, Roxithromycin, Clindamycin, Lincomycin, Amikacin, Gentamicin, Tobramycin, Ciprofloxacin, Levofloxacin, Moxifloxacin, Trimethoprim-Sulfamethoxazole, Doxycycline, Tetracycline, Vancomycin, Teicoplanin, Telavancin, and Linezolid. Exemplary antiviral agents that can be used with the methods of the invention include, but are not limited to, Abacavir, Aciclovir, Acyclovir, Adefovir, Amantadine, Amprenavir, Ampligen, Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Ganciclovir, Ibacitabine, Imunovir, Idoxuridine, Imiquimod, Indinavir, Inosine, Interferon type III, Interferon type II, Interferon type I, Interferon, Lamivudine, Lopinavir, Loviride, Maraviroc, Moroxydine, Methisazone, Nelfinavir, Nevirapine, Nexavir, Nitazoxanide, Novir, Peginterferon alfa-2a, Penciclovir, Peramivir, Pleconaril, Podophyllotoxin, Raltegravir, Ribavirin, Rimantadine, Ritonavir, Pyramidine, Saquinavir, Sofosbuvir, Stavudine, Telaprevir, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir, and Zidovudine. Non-limiting examples of anti-inflammatory agents include non-steroidal anti-inflammatory drugs (NSAIDs), steroidal anti-inflammatory drugs, beta-agonists, anticholingeric agents, and methyl xanthines. Examples of NSAIDs include, but are not limited to, aspirin, ibuprofen, celecoxib, diclofenac, etodolac, fenoprofen, indomethacin, ketoralac, oxaprozin, nabumentone, sulindac, tolmentin, rofecoxib, naproxen, ketoprofen, nabumetone, diclofenac & misoprostol, ibuprofen, ketorolac, valdecoxib, meloxicam, flurbiprofen, and piroxicam. Such NSAIDs function by inhibiting a cyclooxygenase enzyme (e.g., COX-1 and/or COX-2). Examples of steroidal anti-inflammatory drugs include, but are not limited to, glucocorticoids, dexamethasone, cortisone, hydrocortisone, prednisone, prednisolone, triamcinolone, azulfidine, and eicosanoids such as prostaglandins, thromboxanes, and leukotrienes.

In some embodiments, the method comprises further administering an additional anti-cancer treatment modality including, but not limited to, chemotherapy, radiation, surgery, hormonal therapy, or a combination thereof.

Pharmaceutical

The therapeutic and prophylactic methods of the invention thus encompass the use of pharmaceutical compositions. The pharmaceutical compositions useful for practicing the invention may be administered to deliver an effective amount of a therapeutic agent. The precise dosage administered will vary depending upon a number of factors, including but not limited to, the therapeutic agent being administered, the type of animal and type of disease state being treated, the age of the animal and the route of administration.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Other active agents useful in the treatment of fibrosis include anti-inflammatories, including corticosteroids, and immunosuppressants.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, or about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. In one embodiment, such powders comprise particles wherein at least 98% of the particles by weight have a diameter greater than 0.5 nanometers and at least 95% of the particles by number have a diameter less than 7 nanometers. In one embodiment, at least 95% of the particles by weight have a diameter greater than 1 nanometer and at least 90% of the particles by number have a diameter less than 6 nanometers. In some instances, dry powder compositions include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in some instances having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. In one embodiment, the droplets provided by this route of administration have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein. A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. In one embodiment, such powdered, aerosolized, or aerosolized formulations, when dispersed, have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples are not to be construed as limiting in any way the remainder of the disclosure.

### Example 1: CD4 T Cells Provide Antibody Access to Immune-Privileged Tissue

Circulating antibodies can access most tissues to mediate surveillance and elimination of invading pathogens. Immunoprivilaged tissues such as the brain and the peripheral nervous system are shielded from plasma proteins by the blood-brain barrier (Hawkins et al., 2005, Pharmacol. Rev. 57, 173-185) and blood-nerve barrier (Weerasuriya, A. et al., 2011, Methods Mol. Biol. 686, 149-173), respectively. Yet, circulating antibodies must somehow gain access to these tissues to mediate their antimicrobial functions.

It is unclear how antibodies protect against pathogens that enter peripheral tissues devoid of constitutive antibody transport mechanisms. Blood brain barriers consisting of tight junctions between capillary endothelial cells, thick basement membranes and astrocytes' foot processes effectively block the diffusion of antibodies to the brain (Weerasuriya, A. et al., 2011, Methods Mol. Biol. 686, 149-173), while blood nerve barriers consisting of endoneurial vascular endothelium and the perineurium block antibody access to the peripheral neurons (Weerasuriya et al., 2011, Methods Mol Biol 686, 149-173). Such barriers are critical in preventing access by autoreactive antibodies (Milligan, G. N. et al., J. Immunol. 160, 6093-6100). At the same time, because certain pathogens target and replicate within immunoprivilaged sites, a host mechanism to enable directed antibody delivery to these tissues must exist.

These results demonstrate a role of CD4 T cells in controlling antibody access to neuronal tissues through local migration and secretion of IFN-7. Circulating CD4 memory T cells effectively target antibody delivery to the sites of infection through their secretion of IFN-γ, presumably upon recognition of cognate antigenic peptides presented by local antigen-presenting cells (Laidlaw, B. J. et al., 2014, Immunity 41, 633-645, Iijima, N. et al., 2008, J. Exp. Med. 205, 3041-3052). These results indicate the requirement for CD4 T-cell help at the effector phase of the antibody response, and add to the growing appreciation of CD4 T cells in paving the way to other effector cell types such as CD8 T cells (Laidlaw, B. J. et al., 2014, Immunity 41, 633-645, Nakanishi, Y. et al., 2009, Nature 462, 510-513, Reboldi, A. et al., 2009, Nature Immunol. 10, 514-523).

The experimental data demonstrates that the requirement for CD4 T cells for antibody access in neuronal tissue reflects an additional layer of control imposed by the immunoprivilaged sites. In accessible tissues, inflammatory leukocytes can migrate and, in response to PAMPs, secrete cytokines such as TNF-α that are sufficient to trigger vascular permeability independently of CD4 T cells. However, after neurotropic viral infections, the infected neurons are expected to be poor at producing inflammatory cytokines that remodel vascular tight junctions. At the same time, recruitment of innate leukocytes is blocked by shutdown of specific chemokines in the ganglia of HSV-1-infected mice (Stock, A. et al., 2014, J. Exp. Med. 211, 751-759). Curiously, expression of T-cell-trophic chemokines CXCL9 and CXCL10 was preserved in the DRG of infected mice (Stock, A. et al., 2014, J. Exp. Med. 211, 751-759), suggesting that access by lymphocytes is permitted. Thus, in neuronal tissues, the entry of viral-specific CD4 T cells is crucial to provide cytokines that permit antibodies through the induction of vascular permeability.

The results implicate that antibody-based vaccines or treatment against neurotropic viruses would benefit from generating robust circulating CD4 T-cell memory responses.

The materials and methods employed in these experiments are now described.

Mice

Six- to eight-week-old female C57BL/6 (CD45.2⁺) and congenic C57BL/6 B6.SJL-PtprcaPep3b/BoyJ (B6.Ly5.1) (CD45.1⁺) mice, B6.129S2-Ighrc9n/J (MT) mice, anti-HEL B-cell receptor (BCR)-transgenic C57BL/6-TgN (IghelMD4) (HELTg) mice, CBy.PL(B6)-Thy1^{a}/ScrJ (Thy1.1⁺ BALB/c) mice and B6.129X1-Fcgrt^{tm1Dcr}/DcrJ (FcRn^{-/-}) mice were purchased from the National Cancer Institute and Jackson Laboratory. JHD mice (B-cell deficient on BALB/c background) were obtained from Taconic Animal Models.

Viruses

HSV-2 strains 186syn⁻ TK⁻ and 186syn⁺ were obtained. These viruses were propagated and titered on Vero cells (ATCC CCL-81) as previously described (Laidlaw, B. J. et al., 2014, Immunity 41, 633-645). Influenza virus A/Puerto Rico/3/334 (A/PR8: H1N1) and WT/VSV were propagated as previously described (Laidlaw, B. J. et al., 2014, Immunity 41, 633-645, Sasai, M., et al., 2010, Science 329, 1530-1534).

Virus Infection

Six- to eight-week-old female mice injected subcutaneously with Depo Provera (Pharmacia Upjohn, 2 mg per mouse) were immunized intravaginally, intraperitoneally or intranasally with 10⁵ p.f.u. of HSV-2 (186syn-TK-) as previously described (Iijima, N. et al., 2014, Science 346, 93-98). For secondary challenge, immunized mice were challenged vaginally with 10⁴ p.f.u. of WT HSV-2 (186syn⁺) (100% lethal dose for naive mice). In the case of BALB/c and JHD mice, these mice were immunized with 5×10⁴ to 10⁵ p.f.u. of HSV-2. For secondary challenge, immunized mice were challenged with 10⁵ p.f.u. of WT HSV-2 (100% lethal dose for naive mice). The severity of disease was scored as follows: 0, no sign; 1, slight genital erythema and oedema; 2, moderate genital inflammation; 3, purulent genital lesions; 4, hind-limb paralysis; 5, pre-moribund (Laidlaw, B. J. et al., 2014, Immunity 41, 633-645). Owing to humane concerns, the animals were euthanized before reaching moribund state. To measure virus titer in peripheral tissues, vaginal tissues, DRG and spinal cord were harvested in ABC buffer (0.5 mM MgCl₂6H₂O, 0.9 mM CaCl₂2H₂O, 1% glucose, 5% HI FBS and penicillin-streptomycin) including 1% amphotericin-B (Sigma). Thereafter, these tissues were homogenized by lysing matrix D (MP Biomedicals), followed by clarifying by centrifugation. Viral titers were obtained by titration of tissue samples on a Vero cell monolayer. Protein concentration in tissue homogenates was measured by a DC protein assay kit (Bio-Rad Laboratories). C57BL/6 mice were immunized intravenously with WT/VSV (2×10⁶ p.f.u. per mouse) or intranasally with influenza A/PR8 (10 p.f.u. per mouse). For secondary challenge, VSV-immunized mice were re-infected intranasally with WT/VSV (1×10⁷ p.f.u. per mouse).

Antibodies

Anti-CD90.2 (30-H12), anti-CD90.1 (OX-7), anti-CD45.2 (104), anti-CD45.1 (A20), anti-CD4 (GK1.5, RM4-5 and RM4-4), anti-CD19 (6D5), anti-CD45R/B220 (RA3-6B2), anti-MHC class II (I-A/I-E, M5/114.15.2), anti-CD69 (H1.2F3), anti-CD44 (IM7), anti-CD49d (R1-2), anti-NKp46 (29A1.4) and anti-IFN-γ (XMG1.2 and R4-6A2) were purchased from e-Bioscience or Biolegend.

Isolation of Leukocytes from Peripheral Tissues

The genital tracts of vaginal tissues treated with Depo-Provera were dissected from the urethra and cervix. Before collection of neuronal tissues, mice were perfused extensively using transcardiac perfusion and perfusion through inferior vena cava and great saphenous vein with more than 30 ml of PBS. The DRG and the adjacent region of the spinal cord were harvested in PBS for flow cytometry or ABC buffer for tissue homogenization. The tissues in PBS were then incubated with 0.5 mg ml-i Dispase II (Roche) for 15 min at 37° C. Thereafter, vaginal tissues were digested with 1 mg ml⁻¹ collagenase D (Roche) and 30 μg ml⁻¹ DNase I (Sigma-Aldrich) at 37° C. for 25 min. The resulting cells were filtered through a 70-μm filter (Iijima, N. et al., 2011, Proc. Natl Acad. Sci. USA 108, 284-289, Johnson, A. J. et al., 2008, J. Virol. 82, 9678-9688).

Flow Cytometry Preparation of single-cell suspensions from spleen, draining lymph nodes (inguinal lymph node and iliac lymph nodes), vagina and neuronal tissues were described previously. Multiparameter analyses were performed on an LSR II flow cytometer (Becton Dickinson) and analyzed using FlowJo software (Tree Star). To detect HSV-2-specific CD4⁺ T cells or VSV-specific CD4⁺ T cells (CD45.1⁺ or CD45.2⁺), single-cell suspensions from vaginal tissues of TK-HSV-2-immunized mice or VSV immunized mice were stimulated in the presence of 5 μg ml⁻¹ Brefeldin A with naive splenocytes (CD45.1⁺CD45.2⁺) loaded with heat-inactivated HSV-2 antigen, heat-inactivated WT VSV and heat-inactivated influenza virus A/PR8 for around 12 h (Iijima, N. et al., 2014, Science 346, 93-98). To detect HSV-2-specific CD4⁺ T cells in BALB/c and JHD mice, single-cell suspensions (CD90.2⁺) from vaginal tissues of TK-HSV-2-immunized mice were stimulated with naive splenocytes (CD90.1V) loaded with heat-inactivated HSV-2 antigen.

In Vivo Treatment with Neutralizing/Depleting Antibodies

C57BL/6 mice or BALB/c mice were immunized with TK-HSV-2 virus. Five to eight weeks later, these mice were injected intravenously (tail vain) with 300 μg of anti-CD4 (GK1.5; BioXCell) or anti-IFN-γ (XMG1.2; BioXCell) antibody at days −4, −1, 2 and 4 before or after HSV-2 challenge. In vivo depletion for CD4 was confirmed by fluorescence-activated cell sorting analysis of the cell suspension from spleen. For the neutralization of α4-integrin, purified anti-mouse α4 integrin/CD49d (PS/2; SouthernBiotech) was given a tail vain injection of 300 μg antibody at days 2 and 4 after challenge.

Parabiosis

Parabiosis was performed as previously described with slight modifications (Iijima et al., 2014, Science, 346: 93-98). Naive or immunized C57BL/6 mice, HELTg and μMT mice were anaesthetized with a mixture of ketamine/xylazine (100 mg/kg and 10 mg/kg body weight respectively). After shaving the corresponding lateral aspects of each mouse, matching skin incisions were made from behind the ear to hip and sutured together with Chromic Gut (4-0, Henry Schein) absorbable suture, then these areas were clipped with 7-mm stainless-steel wound clips (Roboz).

Measurement of Virus-Specific Ig and Total Ig in Serum and Tissue Homogenates

Ninety-six-well EIA/RIA plates were filled with 100 μl of heat-inactivated purified HSV-2 (10⁴-10⁵ p.f.u. equivalent per 100 μl) or heat-inactivated purified VSV (5×10⁵ p.f.u. equivalent per 100 μl) for virus-specific Ig measurement or goat anti-mouse Ig (1:1,000; SouthernBiotech, 1010-01) for total Ig measurement in carbonate buffer (pH 9.5) and then incubated overnight at 4° C. On the following day, these plates were washed with PBS-Tween 20 and blocked for 2 h with 5% FBS in PBS. Tissue samples and serum samples in ABC buffer were then plated in the wells and incubated for at least 4 h at ambient temperature. After washing in PBS-Tween 20, HRP-conjugated anti-mouse IgG1, IgG3, IgM, IgA, IgG2a, IgG2b or IgG2c (SouthernBiotech) was added to the wells for 1 h, followed by washing and adding TMB solution (eBioscience). Reactions were stopped with 1 N H₂SO₄ and absorbance was measured at 450 nm. The sample antibody titers were defined by using Ig standard (C57BL/6 Mouse Immunoglobulin Panel; SouthernBiotech) or mouse IgG2a (HOPC-1; SouthernBiotech).

Albumin ELISA

Using tissue homogenates (DRG and spinal cord) prepared after extensive perfusion, albumin ELISA (Genway) was performed according to the manufacturer's instructions.

Immunofluorescence Staining

Frozen sections 8 μm in thickness were cut, fixed and left to dry at ambient temperature. These tissues were stained with the antibodies (anti-CD4 (H129.19), anti-MHC class II (M5/114.15.2) anti-VCAM-1 (429/MVCAM.A), anti-CD31 (390 and MEC13.3), anti-Ly6G (1A8), anti-CD11b (M1/70) and anti-mouse albumin (Goat pAb/Bethyl Laboratories) as previously described (Iijima, N. et al., 2014, Science 346, 93-98). These slides were washed and incubated with DAPI and mounted with Fluoromount-G (SouthernBiotech). They were analyzed by fluorescence microscopy (BX51; Olympus).

Vascular Permeability Assays

Spinal column was harvested from intranasal TK⁻HSV-2-immunized mice 45 min after tail vein injection with 200 μl of 5 mg ml⁻¹ Oregon Green 488-conjugated dextran (70 kDa, D7173, Thermo Fisher Scientific) in PBS. Spine was then fixed with 4% paraformaldehyde in PBS overnight, and frozen sections cut (8 μm in thickness) for immunohistochemical analysis (Knowland, D. et al., 2014, Neuron 82, 603-617).

DNA Isolation from Tissues

C57BL/6 mice were immunized intranasally with TK-HSV-2. Six weeks later, vaginal tissues, DRG and spinal cord of these mice were lysed in 10 mg ml⁻¹ Proteinase K (Roche) to isolate DNA at 55° C. overnight. After removing these tubes, phenol equilibrated with Tris pH 8.0 was added. Thereafter, upper aqueous phase was added to phenol/chloroform (1:1). The upper aqueous phase was re-suspended with sodium acetate, pH 6.0, and 100% ethanol at room temperature. After shaking and centrifuging, the concentration of isolated DNA pellet was measured. The level of HSV-2 genomic DNA in peripheral tissues on the basis of HSV-2 gD (forward primer: AGCGAGGATAACCTGGGATT (SEQ ID NO: 1); reverse primer: GGGATAAAGCGGGGTAACAT (SEQ ID NO: 2)) was analyzed by quantitative PCR using purified viral DNA genome as standard.

Statistical Analysis

Survival curves were analyzed using a log-rank test. For other data, normally distributed continuous variable comparisons used a two-tailed unpaired Student's t-test or paired Student's t-test with Prism software. To compare two non-parametric data sets, a Mann-Whitney U-test was used.

The results of the experiments are now described.

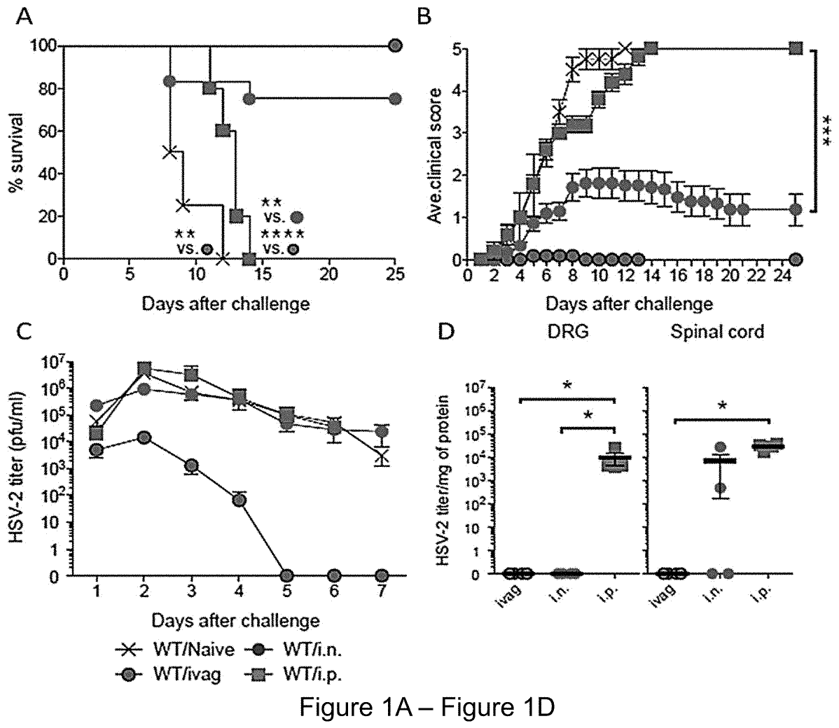

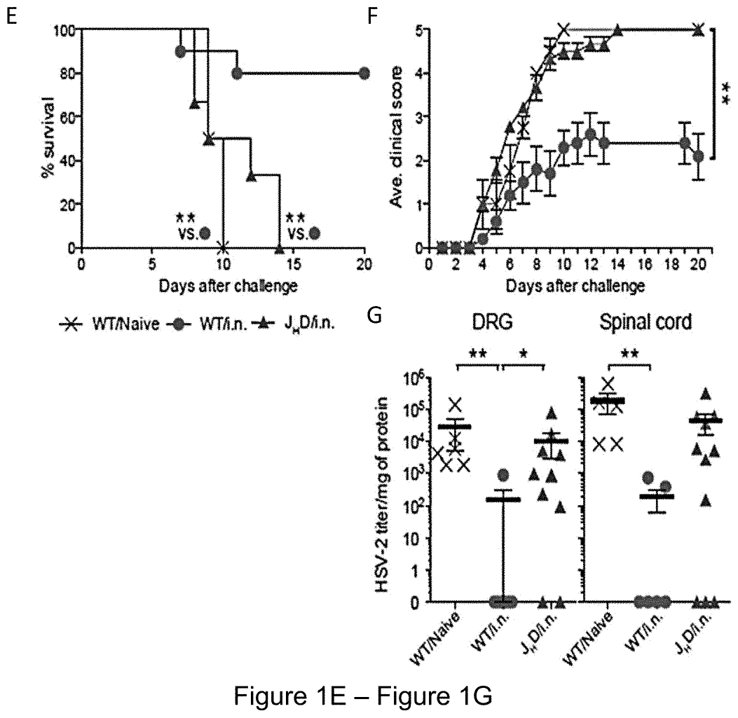

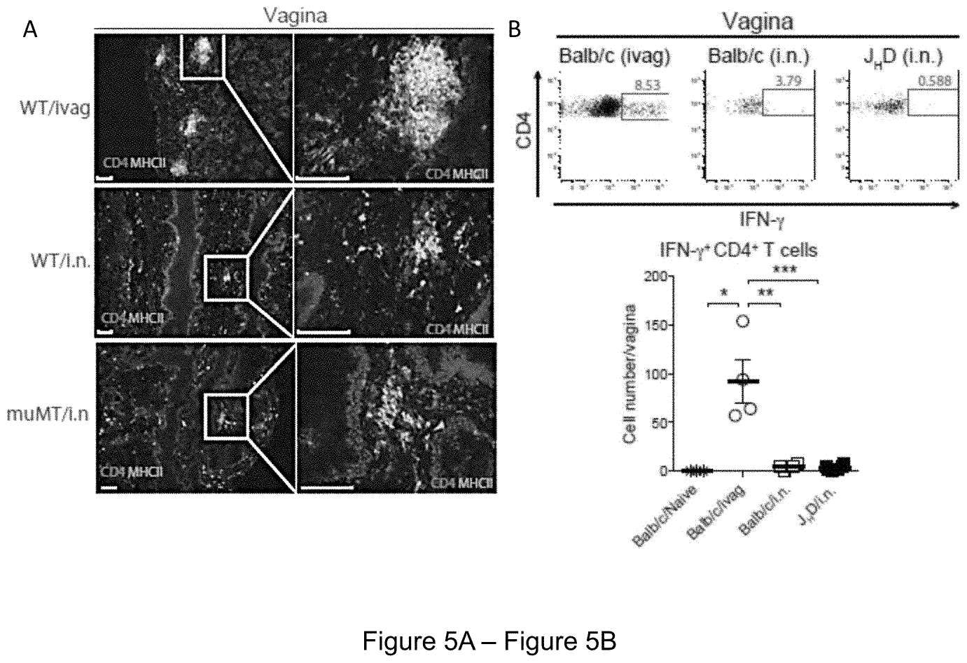

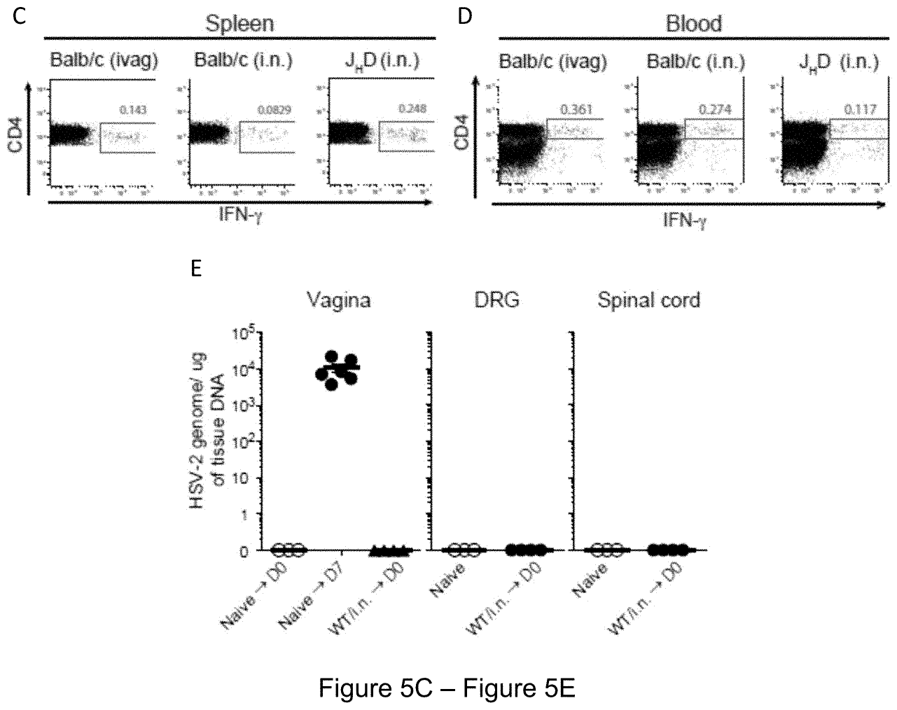

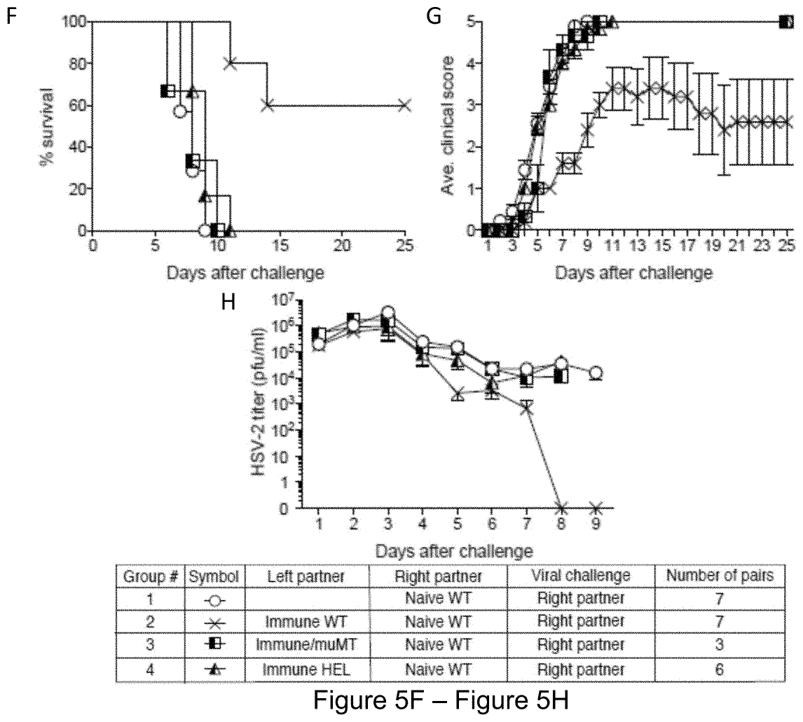

To investigate the mechanism of antibody-mediated protection within the barrier-protected tissues, a mouse model of genital herpes infection was used. Herpes simplex virus type 2 (HSV-2) enters the host through the mucosal epithelia, and infects the innervating neurons in the dorsal root ganglia (DRG) to establish latency (Koelle, D. M. et al., 2008, Annu. Rev. Med. 59, 381-395, Knipe, D. M. et al., 2008 Nature Rev. Microbiol. 6, 211-221). Vaginal immunization by an attenuated HSV-2 with deletion of the thymidine kinase gene (TK-HSV-2) provides complete protection from lethal disease following genital challenge with wild-type (WT) HSV-2 (Parr, M. B. et al., 1994, Lab. Invest. 70, 369-380) by establishing tissue-resident memory T cells (TRM) (Iijima, N. et al., 2014, Science 346, 93-98). In vaginally immunized mice, interferon (IFN)-y-secretion by CD4 T cells, but not antibodies, are required for protection (Milligan, G. N. et al., 1998, J. Immunol. 160, 6093-6100, Parr, M. B. et al., 2000, Immunology 101, 126-131). In contrast, distal immunization with the same virus fails to establish TRM and provides only partial protection (Iijima, N. et al., 2014, 2014, Science 346, 93-98). Nevertheless, of the distal immunization routes tested, intranasal immunization with TK-HSV-2 provided the most robust protection against intravaginal challenge with WT HSV-2, whereas intraperitoneal immunization provided the least protection (FIG. 1A through FIG. 1D) (Sato, A. et al., 2014, J. Virol. 88, 13699-13708, Jones, C. A. et al., 2000, Virology 278, 137-150). As shown previously (Iijima, N. et al., 2014, Science 346, 93-98), intranasal immunization did not establish TRM in the genital mucosa (FIG. 5A, FIG. 5B), despite generating a comparable circulating memory T-cell pool (FIG. 5C, FIG. 5D). After vaginal HSV-2 challenge, mice that were immunized intranasally with TK-HSV-2 were unable to control viral replication within the vaginal mucosa (FIG. 1C), but had significantly reduced viral replication in the innervating neurons of the DRG (FIG. 1D). Notably, it was found that protection conferred by intranasal immunization required B cells, as JHD mice (deficient in B cells) were not protected by intranasal immunization (FIG. 1E-FIG. 1G). In the absence of B cells, intranasal immunization was unable to control viral replication in the DRG and spinal cord (FIG. 1G).

In mice immunized intranasally with TK-HSV-2, no evidence of infection in the DRG or the spinal cord was found (FIG. 5E). Moreover, the intranasal route of immunization was not unique in conferring protective response, as parabiotic mice sharing circulation with intravaginally immunized partners were also partly protected from vaginal challenge with WT HSV-2 in the absence of TRM (Iijima, N. et al., 2014, Science 346, 93-98) (FIG. 5F-FIG. 5H). It was found that the B cells in the immunized partners were required to confer protection in the naive conjoined mice, as partners of immunized μMT mice were unprotected (FIG. 5F-FIG. 5H). Moreover, antigen-specific B cells were required to confer protection, as intravaginally immunized partners whose B cells bore an irrelevant B cell receptor (against hen egg lysozyme (HEL)) were unable to confer protection in the conjoined naive partner (FIG. 5F-FIG. 5H). As observed for the intranasal immunization, viral control conferred by the immunized parabiotic partner was not observed in the vaginal mucosa (FIG. 5H), demonstrating that protection occurs in the innervating neurons.

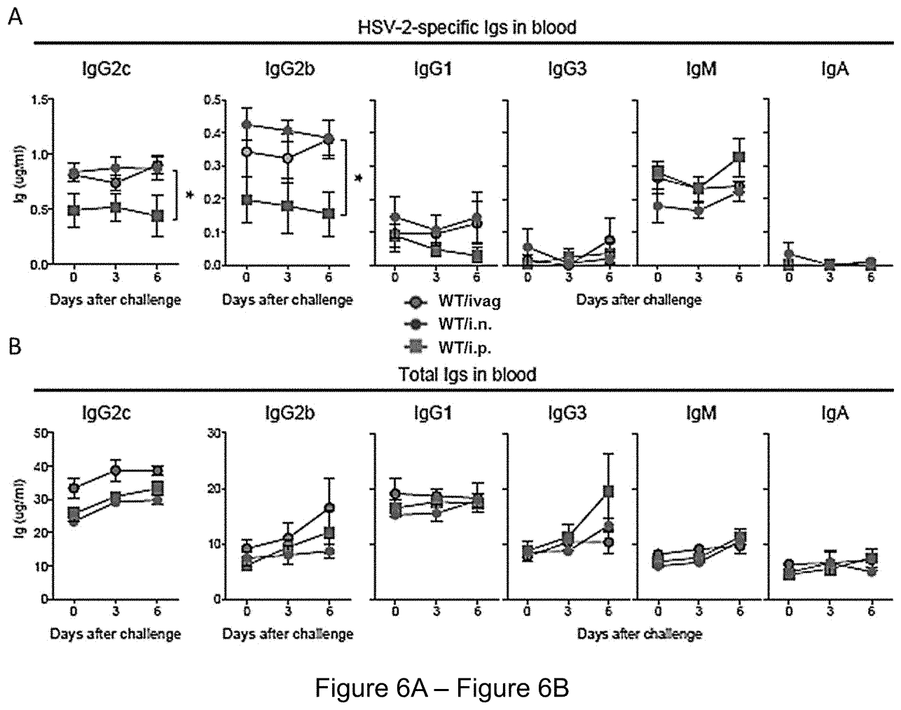

Next, the basis for superior protection by antibodies following different routes of immunization was investigated. Intravaginal, intranasal and intraperitoneal routes of immunization with TK-HSV-2 results in comparable circulating CD4 T-cell memory responses (Iijima, N. et al., 2014, Science 346, 93-98). While no differences were seen for other isotypes, the intranasal and intravaginal routes of immunization were superior to intraperitoneal route in generating higher levels of systemic HSV-2-specific immunoglobulin-G (IgG)2b and IgG2c responses (FIG. 6). These results indicated that higher levels of circulating virus-specific IgG2b and IgG2c correlate with protection against vaginal HSV-2 challenge.

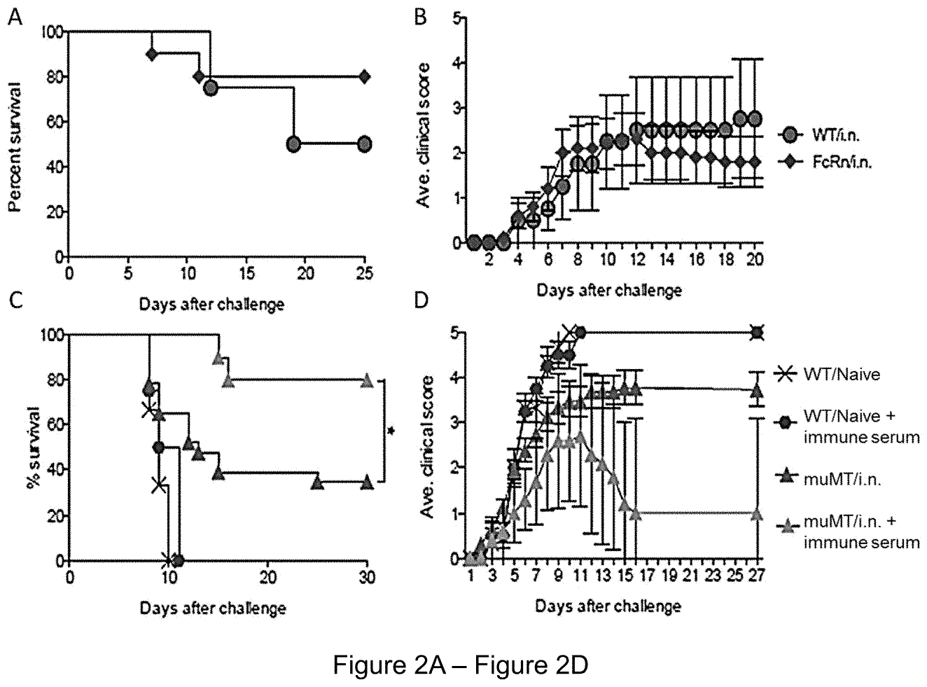

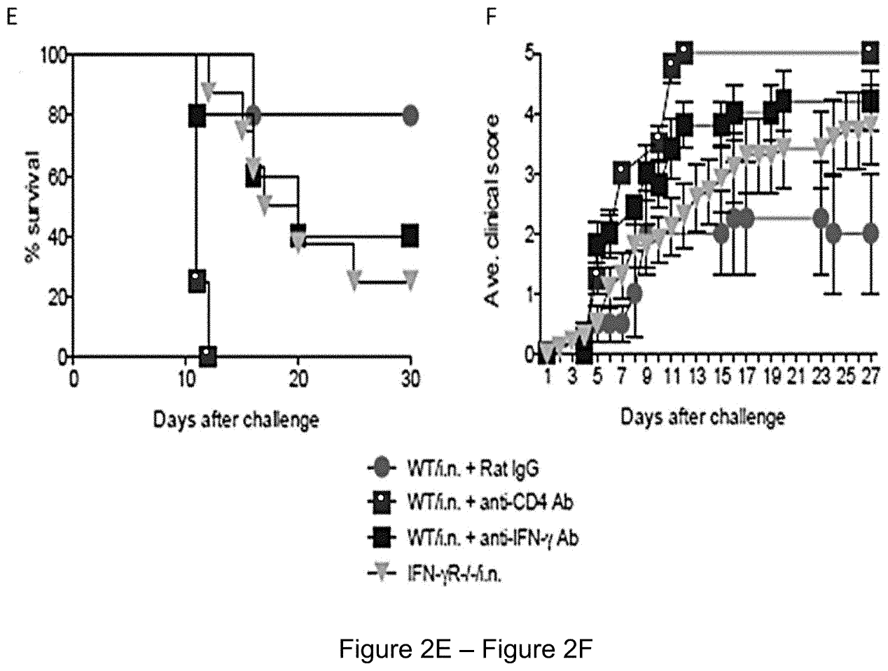

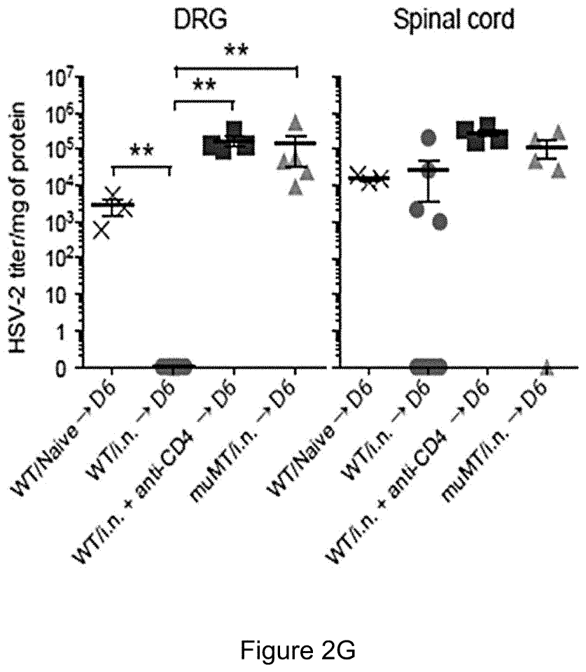

It was next determined how antibody access to the DRG and spinal cord is mediated. Even though the peripheral nervous tissues are protected from antibody diffusion through the blood-nerve barrier, it was formally possible that secretion of antibody into the tissue occurs through transport of serum antibody by the neonatal Fc receptor for IgG (FcRn) (Roopenian, D. C. et al., 2007, Nature Rev. Immunol. 7, 715-725) expressed on the endothelial cells within the infected tissues. However, it was found that mice deficient in FcRn immunized intranasally with TK-HSV-2 were equally protected as the WT counterpart from vaginal HSV-2 infection (FIG. 2A and FIG. 2B). Thus, circulating HSV-2-specific antibodies are somehow mobilized to the neuronal tissues following local viral infection in an FcRn-independent manner, and are required for protection of the host.

If circulating antibodies are sufficient, passive transfer of HSV-2-specific antibodies alone should be able to protect the host. However, it has been shown (McDermott, M. R. et al., 1990, J. Gen. Virol. 71, 1497-1504, Morrison, L. A. et al., 2001 J. Virol. 75, 1195-1204) that intravenous injection of HSV-2-specific antibodies alone fails to protect naive mice against HSV-2 challenge (FIG. 2C and FIG. 2D). In contrast, consistent with a previous study (Morrison, L. A. et al., 2001, J. Virol. 75, 1195-1204), it was discovered that B-cell-deficient μMT mice immunized intranasally with TK-HSV-2 and given systemic administration of HSV-2-specific antiserum were protected (FIG. 2C and FIG. 2D). Thus, these results demonstrate that it is the secreted antibodies, and not B cells themselves, in concert with non-B-cell immune cells, probably T cells induced by immunization, that seem to be required for protection. To test this possibility, CD4 T cells from mice previously immunized were depleted intranasally just before intravaginal HSV-2 challenge. In this setting, differentiation of B cells and antibody responses were allowed to occur fully in the presence of CD4 T-cell help for 6 weeks. Mice acutely depleted of CD4 T cells succumbed to infection with HSV-2 (FIG. 2E and FIG. 2F), whereas depletion of CD8 T cells and natural killer (NK) cells had no effect (Sato, A. et al., 2014, J. Virol. 88, 13699-13708). Moreover, neutralization of IFN-γ before challenge, or genetic deficiency in IFN-γR, also rendered intranasally immunized mice more susceptible to intravaginal HSV-2 challenge (FIG. 2E and FIG. 2F). Of note, depletion of CD4 T cells from intranasally immunized mice just before the viral challenge rendered mice incapable of viral control in the DRG, to a similar extent as the immunized B-cell-deficient μMT mice (FIG. 2G). It was observed that intranasal immunization conferred near-complete protection from HSV-2 in the DRG but variable protection in the spinal cord (FIG. 1D and FIG. 2G). Because HSV-2 can differentially seed the DRG and spinal cord through sensory neurons and autonomic neurons (Ohashi, M. et al., 2011, J. Virol. 85, 3030-3032), these data demonstrate that the efficacy of antibody-mediated protection may depend on the route of viral entry. Further, these results indicate that circulating antibodies, CD4 T cells and IFN-γ collectively mediate neuroprotection against HSV-2.

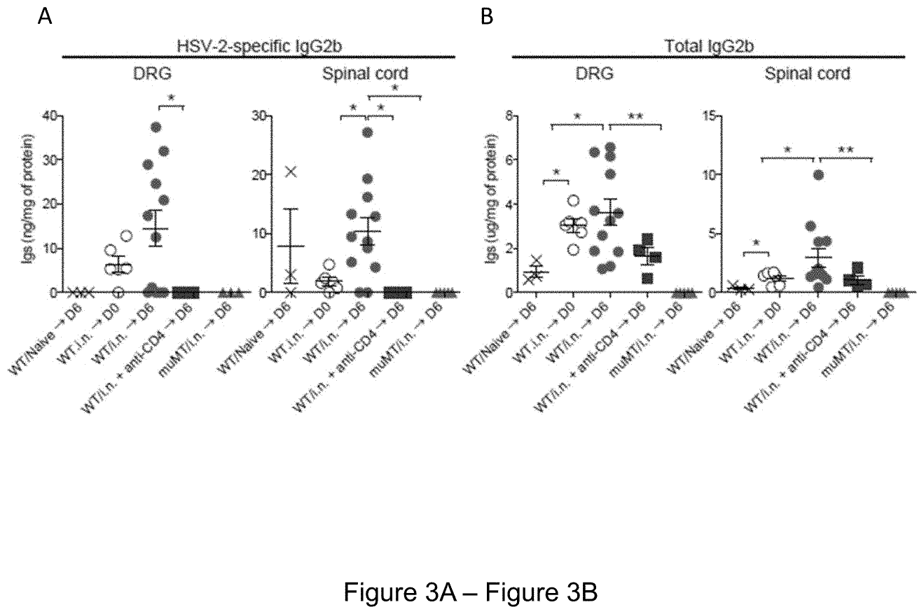

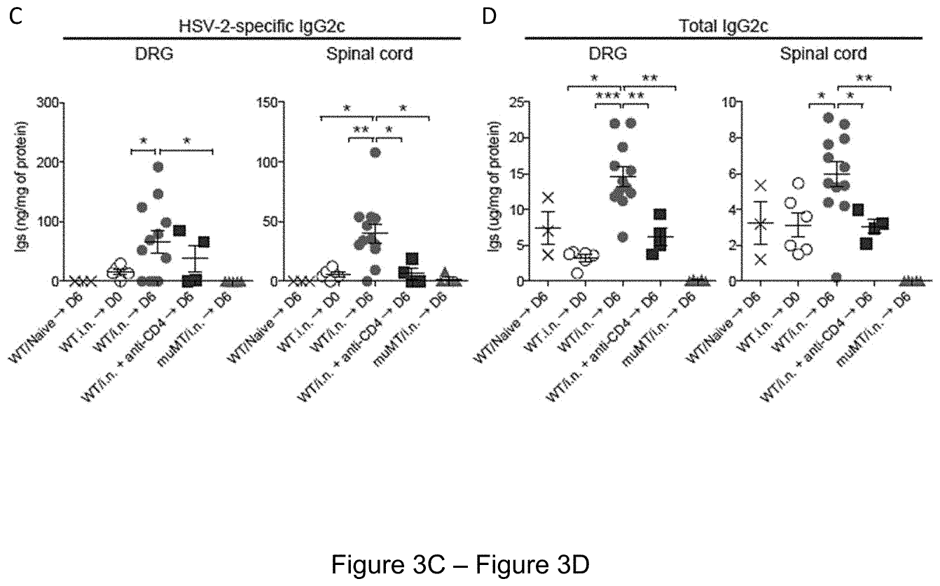

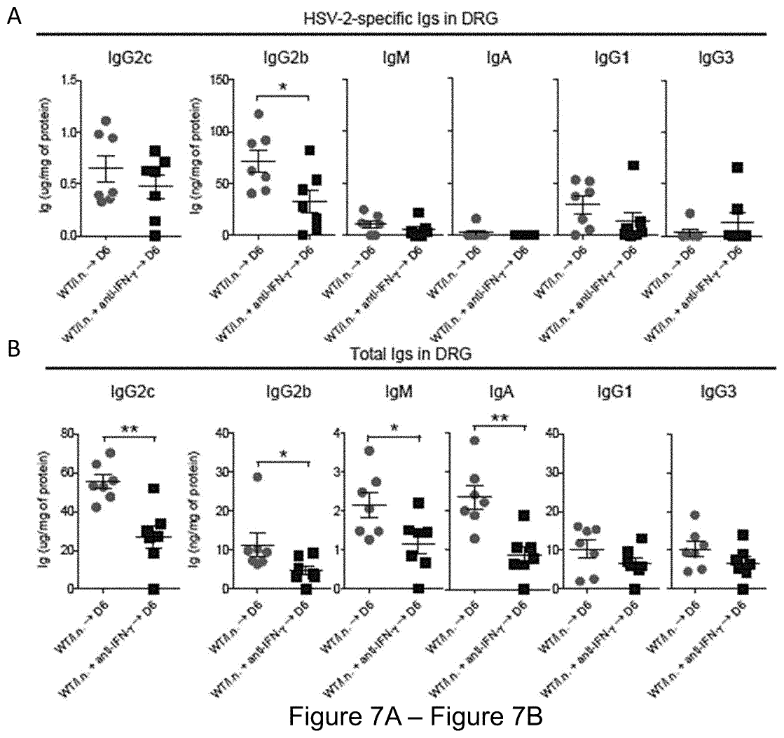

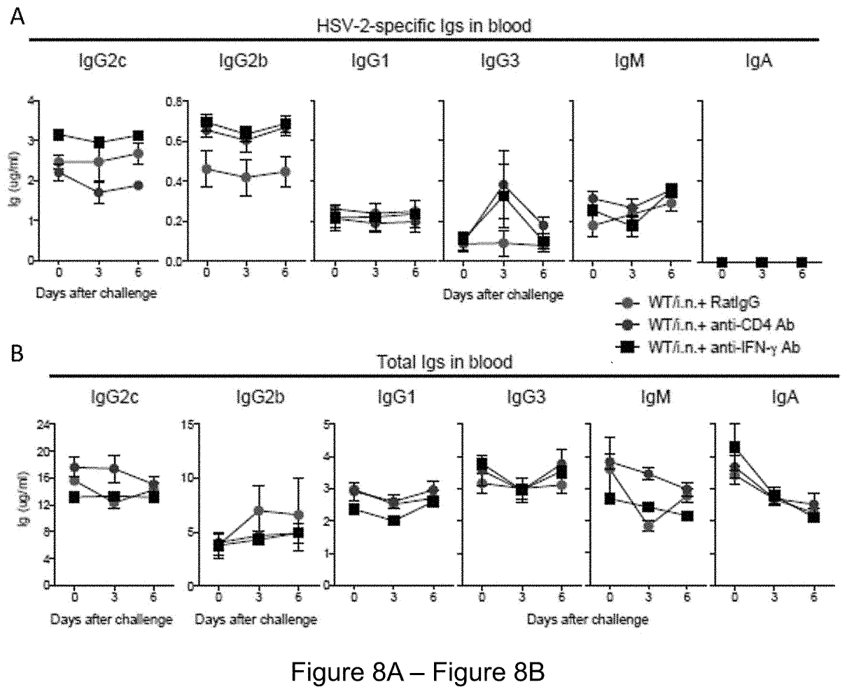

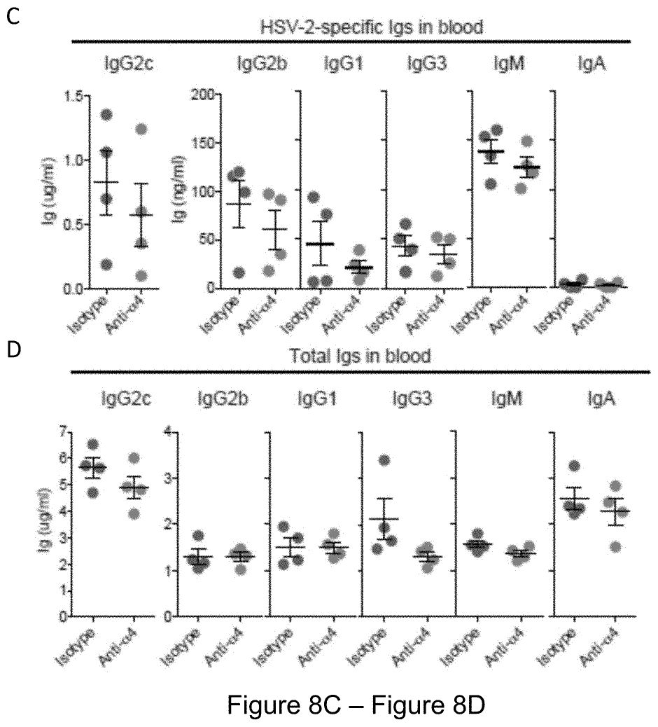

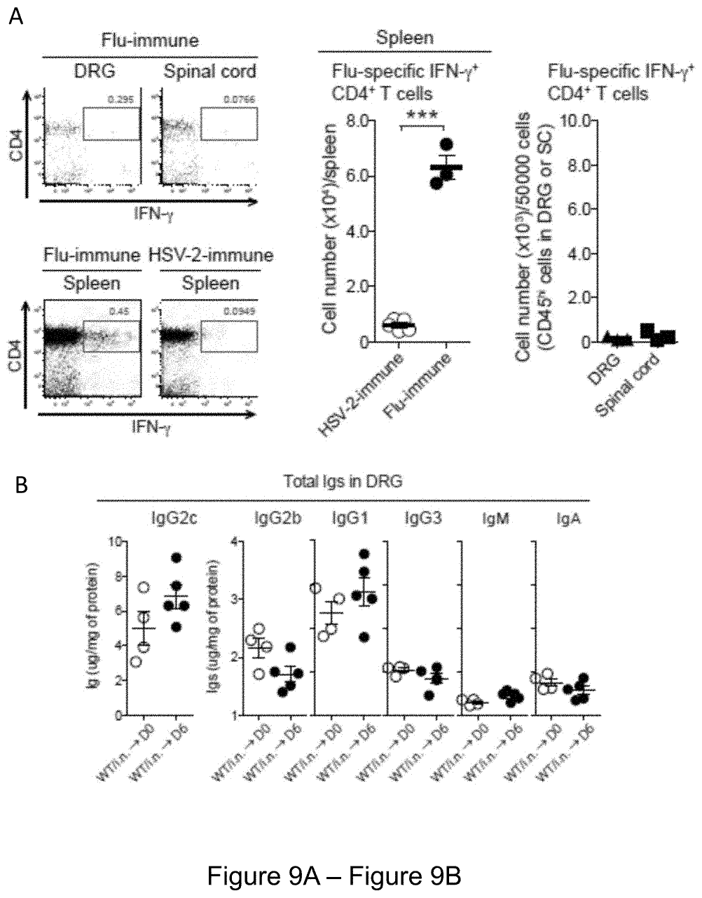

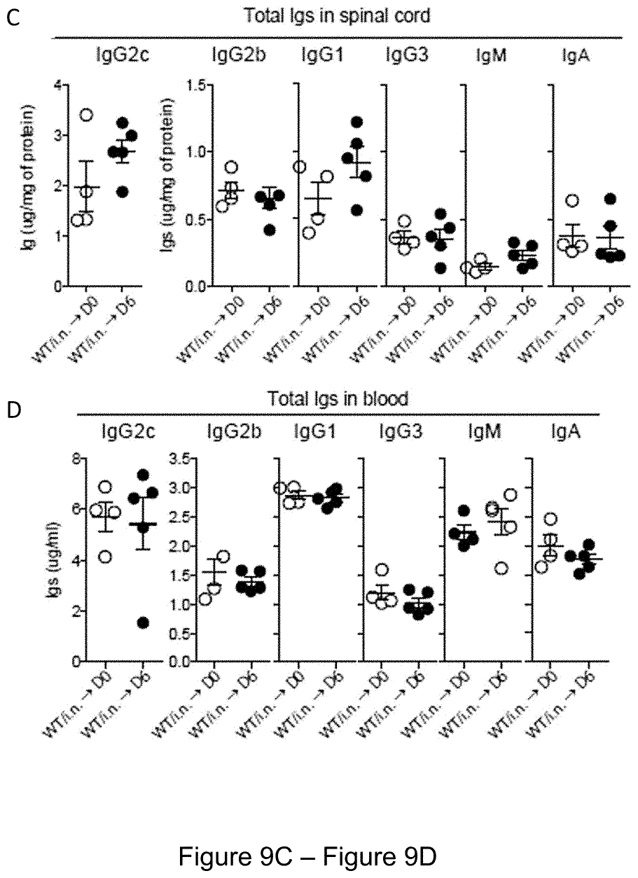

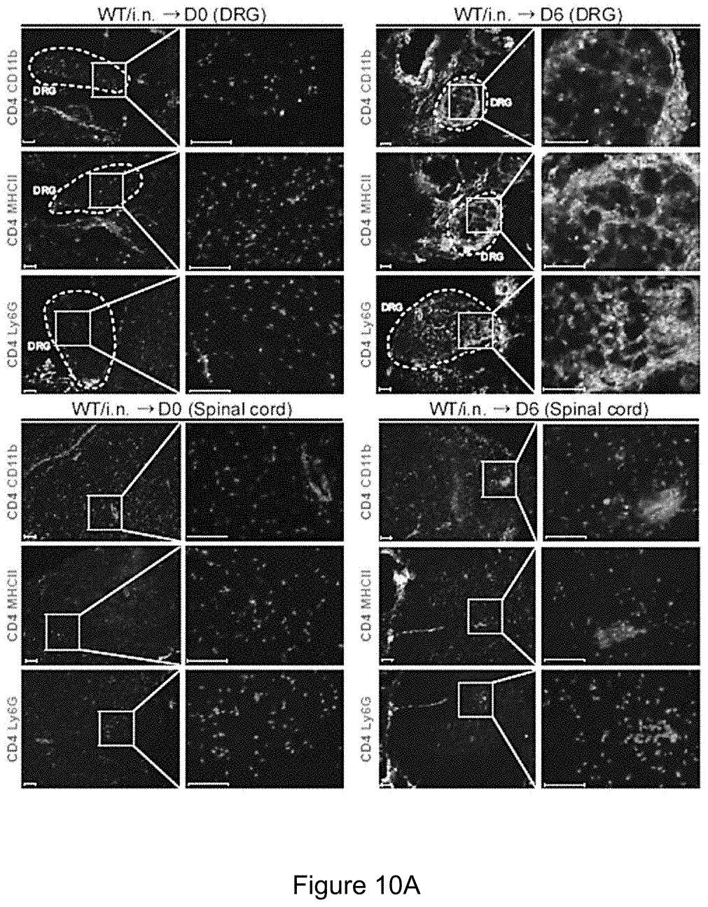

Given that antibody-mediated protection occurs at the level of the innervating neurons and not within the vagina (FIG. 1C and FIG. 5H), it is hypothesized that CD4 T cells will control delivery of antibodies to the tissue parenchyma through secretion of IFN-γ. Low levels of virus-specific and total antibodies were detected in the DRG or spinal cord at steady state in immunized mice (FIG. 3; WT/intranasally →DO), and undetectable levels of antibodies in these tissues in previously unimmunized mice 6 days after an acute infection with HSV-2 (FIG. 3; WT/naive →D6). However, in mice immunized intranasally with TK-HSV-2 6 weeks earlier, increase in the levels of antibodies was detected 6 days after intravaginal HSV-2 challenge within the DRG and in the spinal cord (FIG. 3; WT/intranasally →D6). Moreover, CD4 T cells were required for access of virus-specific antibodies to the restricted tissue such as the DRG, as depletion of CD4 T cells completely diminished antibody levels in this tissue and spinal cord (FIG. 3D; WT/intranasally+anti-CD4-D6). Further, similar requirement for CD4 T cells (Figure. 3B, Figure D) and IFN-γ (FIG. 7) was found for diffusion of total IgG2b and IgG2c isotypes into the DRG, demonstrating that the delivery mechanism does not discriminate virus-specificity of the antibodies. In contrast to the neuronal tissues, acute depletion of CD4 or IFN-γ blockade once antibody responses were established had no significant impact on the serum levels of anti-HSV-2 or total antibodies (FIG. 8A and FIG. 8B). To determine whether antigen-specific memory CD4 T cells were required to mediate antibody access to the neuronal tissues, mice were primed intranasally with a heterologous virus, influenza A virus and, 4 weeks later, were challenged with HSV-2 intravaginally. In contrast to mice harboring cognate memory CD4 T cells, antibody access to neuronal tissues following intravaginal HSV-2 challenge was not observed in mice that had irrelevant memory CD4 T cells (against influenza A virus) (FIG. 9). These data indicate that antigen-specific memory CD4 T cells are required for antibody access to the neuronal tissues.

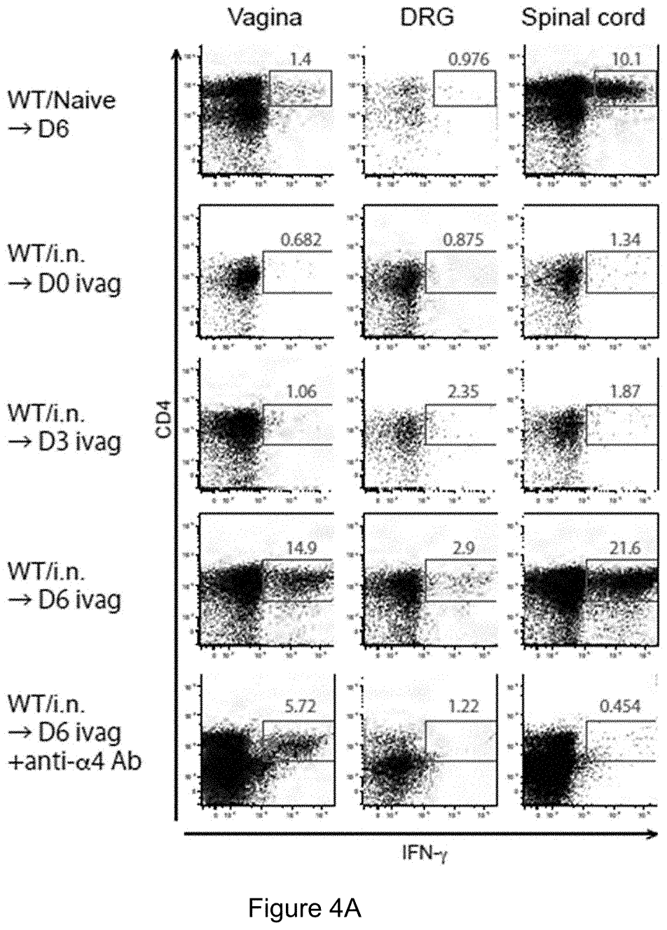

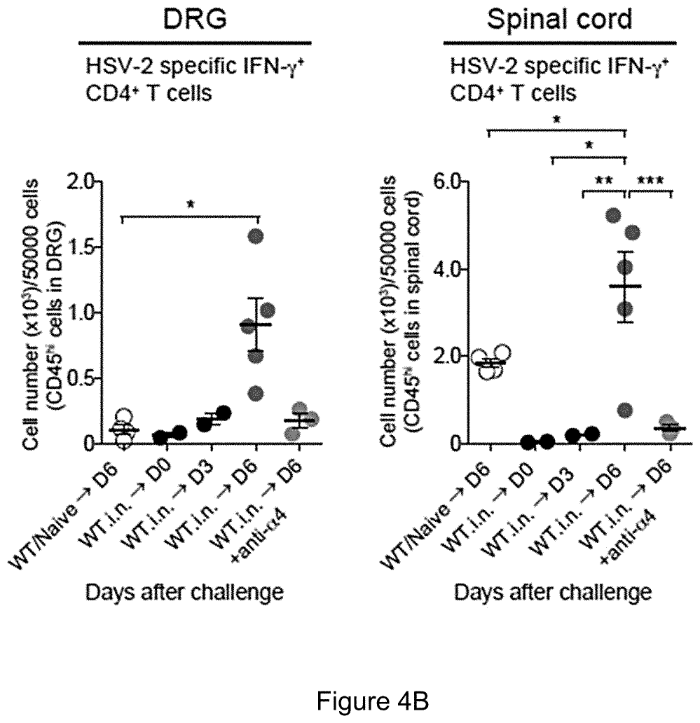

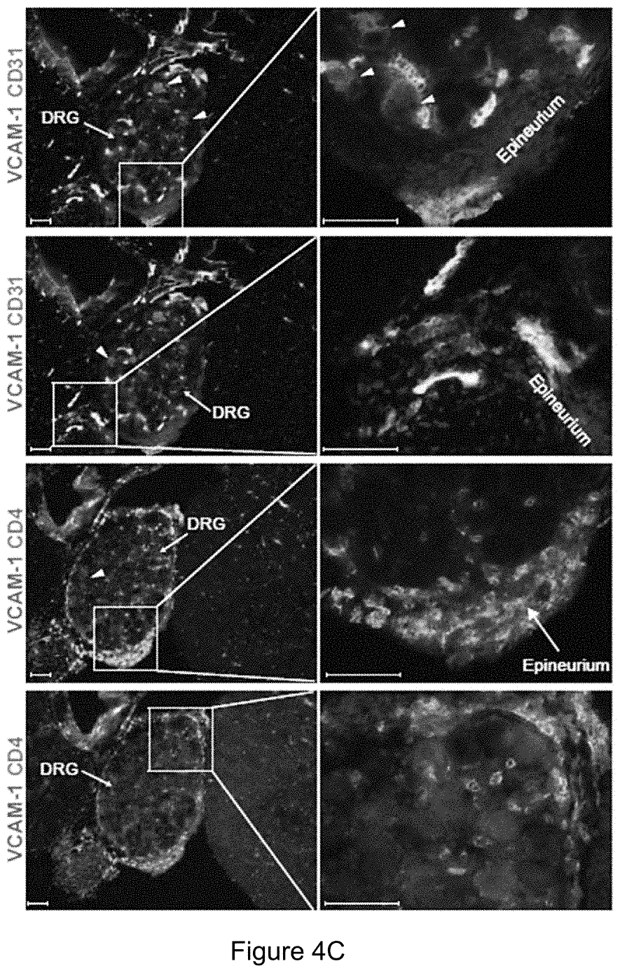

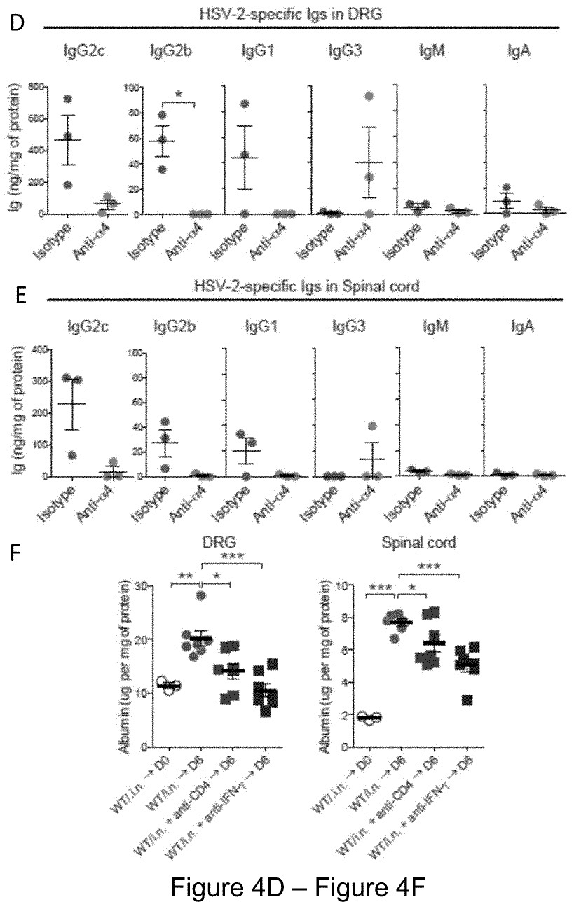

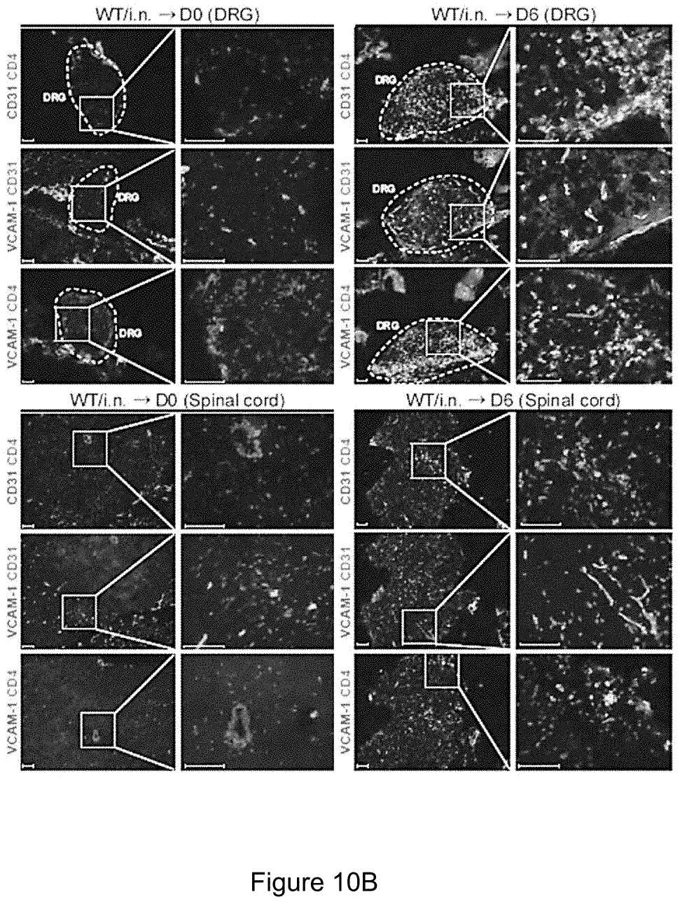

It was hypothesized that memory CD4 T cell might enter the barrier-protected tissues and mobilize antibody access through local secretion of IFN-7. In support of this idea, it was found that IFN-γ-secreting HSV-2-specific CD4 T cells entered the DRG and spinal cord around 6 days after genital HSV-2 challenge in mice that received intranasal immunization 6 weeks previously (FIG. 4A and FIG. 4B; WT/intranasally →D6). Some increase in innate leukocytes bearing CD11b, Ly6G or MHCII was observed in DRG and spinal cord 6 days after challenge (FIG. 10A). IFN-γ secretion was confined to the memory CD4 T-cell population within the DRG (FIG. 4A). Moreover, entry of effector CD4 T cells to the DRG and spinal cord at 6 days after primary vaginal HSV-2 infection was much less efficient than their memory counterpart (FIG. 4A and FIG. 4B; WT/naive →D6), demonstrating that the intrinsic ability of T cells to migrate into these neuronal tissues is enhanced with memory development.

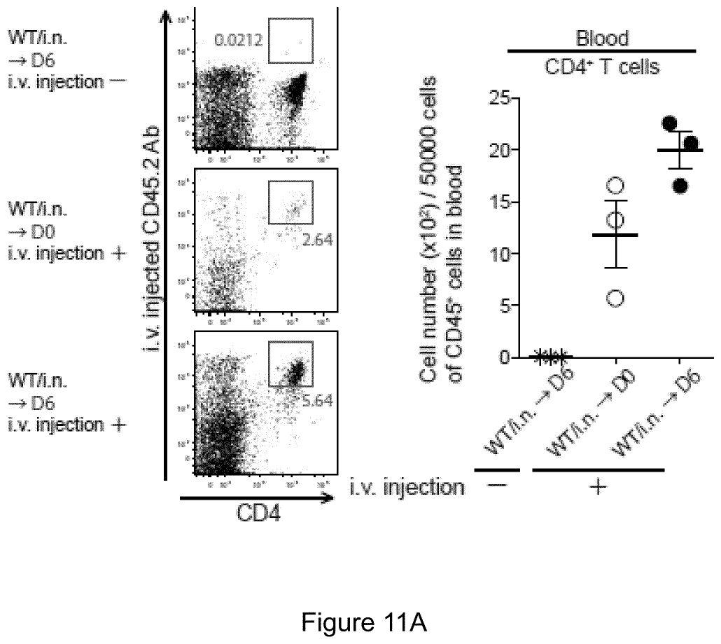

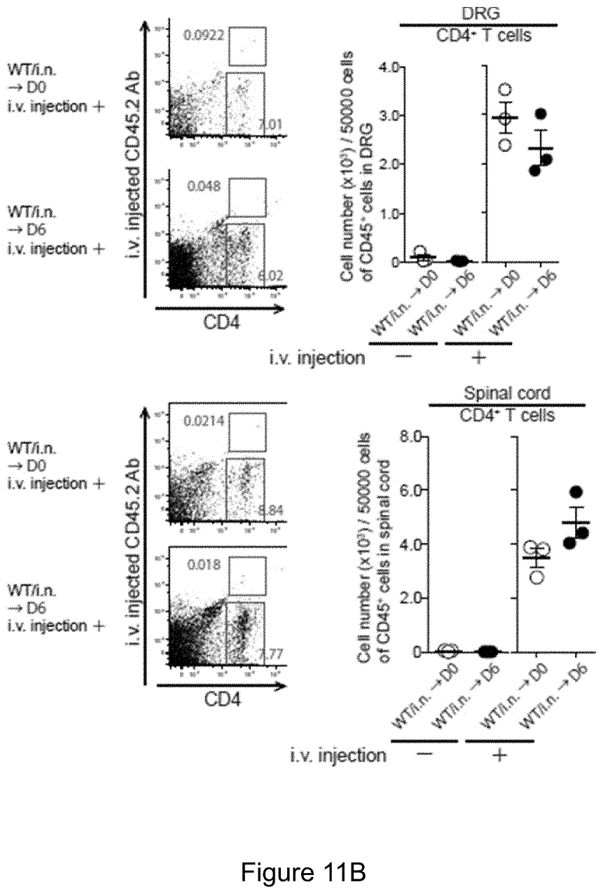

Interaction of α4β1 (or VLA4) and VCAM-1 contributes to T-cell recruitment across the blood-brain barrier (Man, S. et al., 2007, Brain Pathol. 17, 243-250). Memory CD4 T cells generated against HSV-2 expresses CD49d which is the integrin α4 subunit (Iijima, N. et al., 2014, Science 346, 93-98). It was found that the entry of memory CD4 T cells into the nervous tissue was strictly dependent on α4 integrin, as antibody blockade of α4 prevented their entry into the DRG and spinal cord (FIG. 4A and FIG. 4B). The expression of ligand for α4β1, VCAM-1, was observed in the endothelium of DRG and spinal cord in immune-challenged mice (FIG. 4C and FIG. 10B). Further, analysis of tissue sections revealed that the CD4 T cells were found in the parenchyma of the DRG and spinal cord, as well as within their epineurium and meninges, but not within the vasculature (FIG. 4C, FIG. 10A and FIG. 10B). Notably, many CD4 T cells were found adjacent to the cell body of neurons within the DRG. Some VCAM-1 staining was found in the cytosol of neuronal cell bodies (arrowhead FIG. 4C). Additionally, intravascular staining (Anderson, K. G. et al., 2014. Nature Protocols 9, 209-222) with antibody to CD90.2 revealed that the vast majority of the CD4 T cells in the DRG and spinal cord are sequestered from circulation (FIG. 11A, FIG. 11B). Thus, CD4 T cells recruited to the neuronal tissues access the parenchyma of the DRG and spinal cord. Notably, α4 integrin blockade of CD4 T-cell recruitment resulted in diminished access of virus-specific antibody to the DRG and spinal cord (FIG. 4D and FIG. 4E), with no effect on blood levels of virus-specific antibody (FIG. 8C) or the total antibody levels of various isotypes in circulation (FIG. 8D). Collectively, these data indicate that memory CD4 T cells enter the neuronal tissue and secrete IFN-γ to promote antibody access to the DRG and spinal cord.

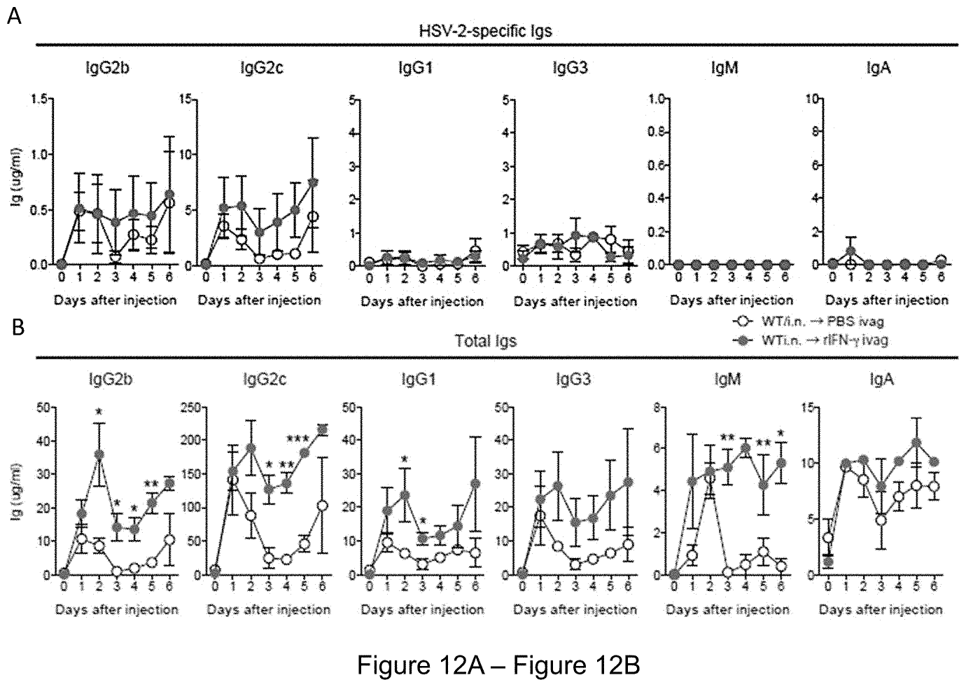

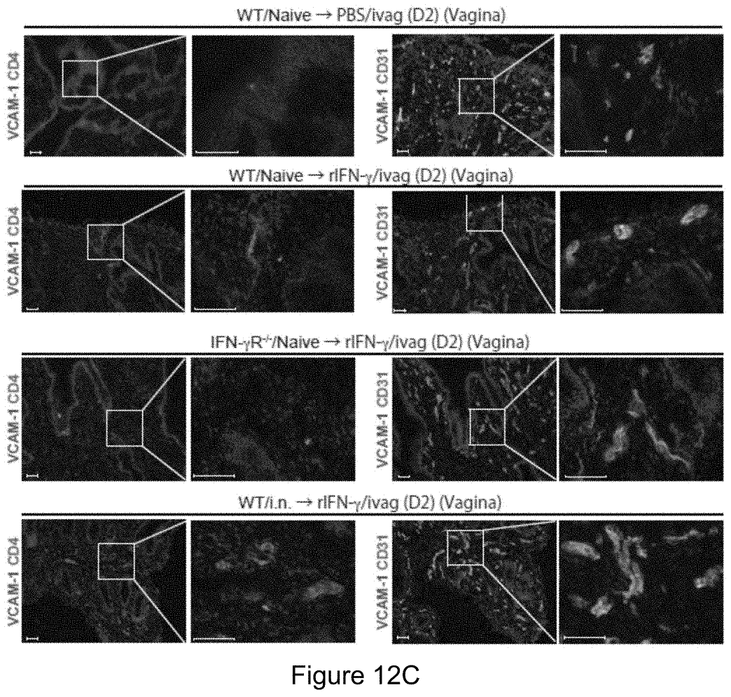

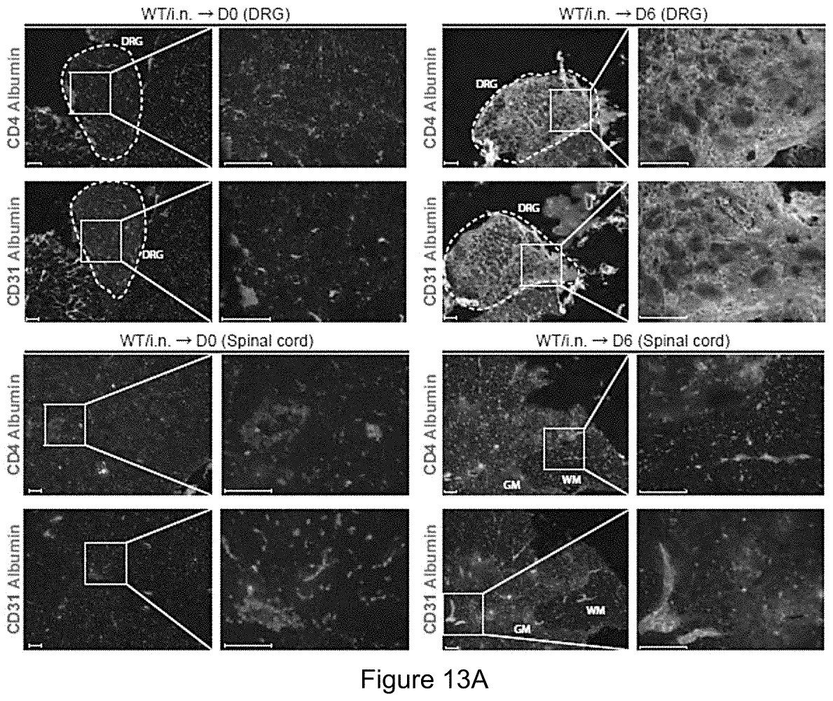

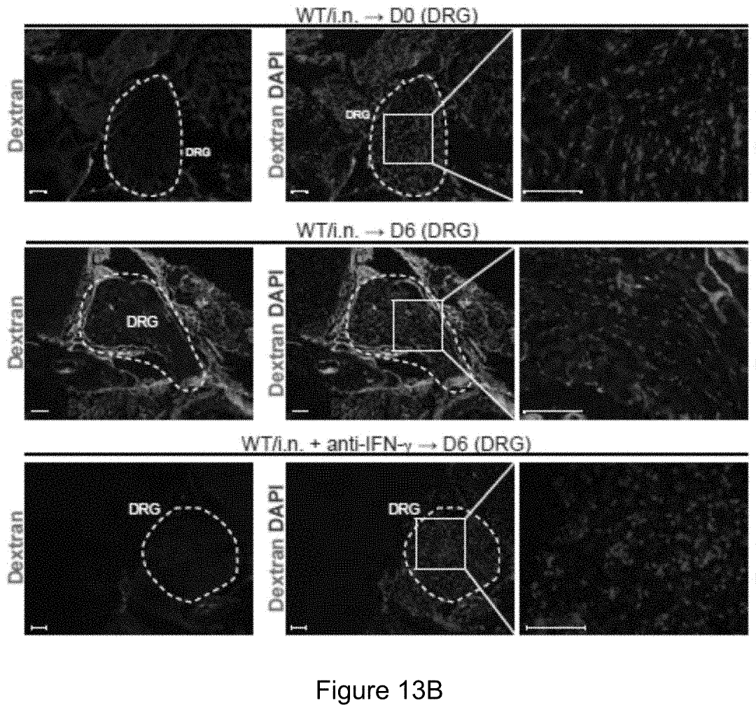

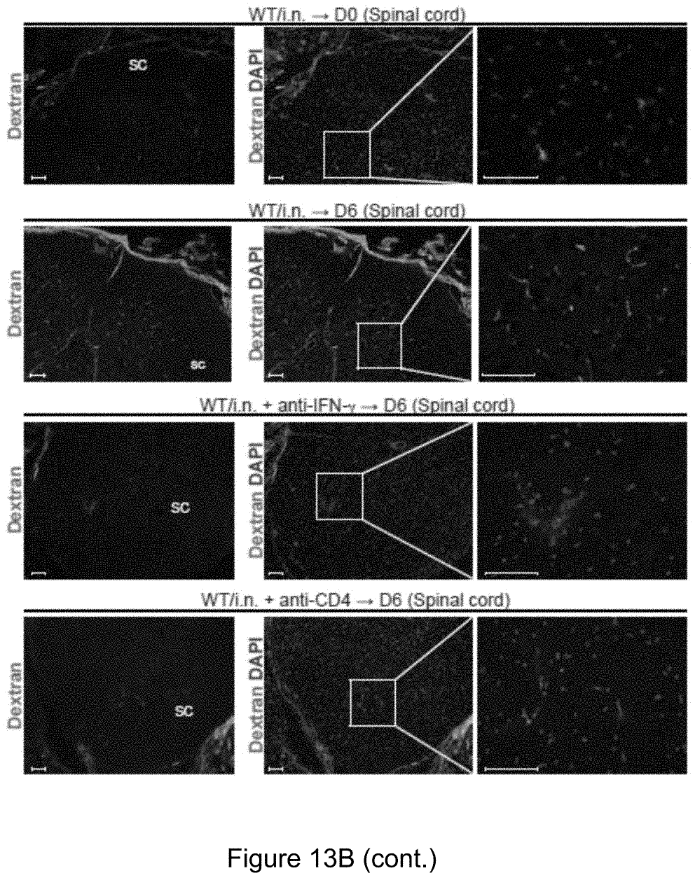

How might IFN-γ secreted by CD4 T cells enable circulating antibody to access the neuronal tissues? IFN-γ acts on the endothelial cells to remodel tight junctions and increase permeability (Capaldo, C. T. et al. 2014, Mol. Biol. Cell 25, 2710-2719). It was observed that recombinant IFN-γ injected intravaginally was sufficient to enable antibody access to the vaginal lumen, suggesting that IFN-γ is sufficient to induce both vascular and epithelial permeability in peripheral tissues (FIG. 12A) and to enhance VCAM-1 expression on endothelial cells (FIG. 12B). To assess whether antibody access to the neuronal tissues mediated by CD4 T cells and IFN-γ is through increased vascular permeability, the measured release of blood albumin into the neuronal tissue following genital HSV-2 challenge in intranasally immunized mice was demonstrated. Notably, it was observed that vascular permeability occurred in the DRG and spinal cord in a CD4 T-cell- and IFN-γ-dependent manner, as measured by leakage of blood albumin to the neuronal tissues by ELISA and immunohistochemical analysis (FIG. 4F and Figure. 13A). It was confirmed that CD4-dependent vascular permeability to the DRG and the spinal cord using intravenous injection of 70 kDa fluorescein isothiocyanate (FITC)-dextran, which has a similar size to IgG (FIG. 13B). Collectively, the results support the notion that CD4 T cells enable antibody delivery to the sites of infection by secreting IFN-γ and enhancing microvascular permeability. This mechanism of antibody delivery is crucial for host immune protection, as depletion of CD4 T cells, inhibition of CD4 T-cell migration into the neuronal tissues or neutralization of IFN-γ renders immune mice susceptible to infection.

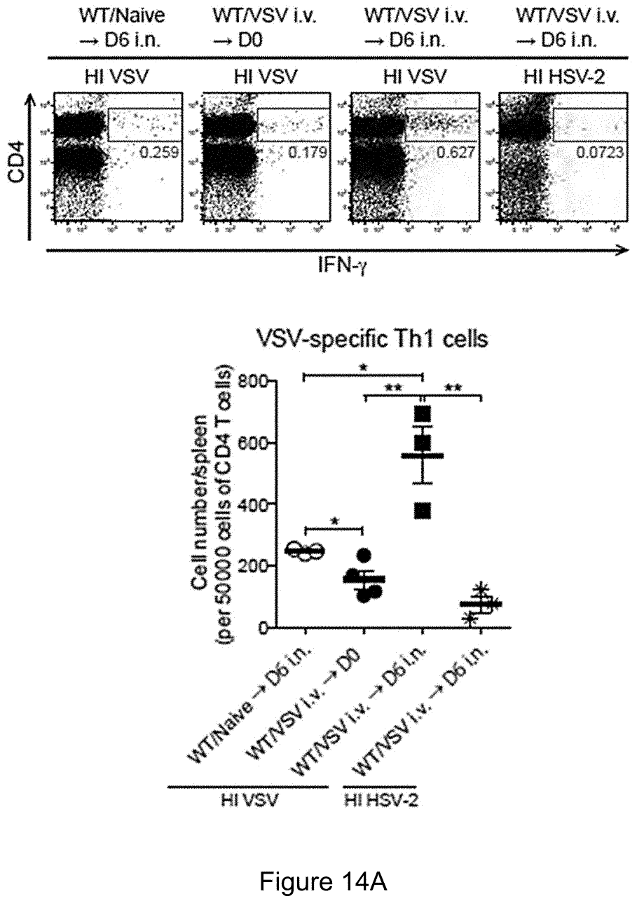

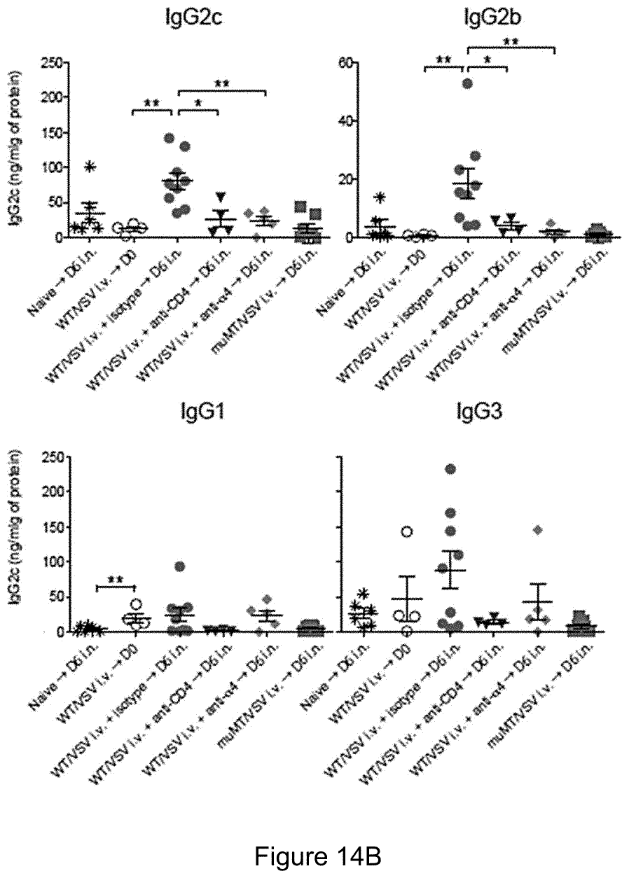

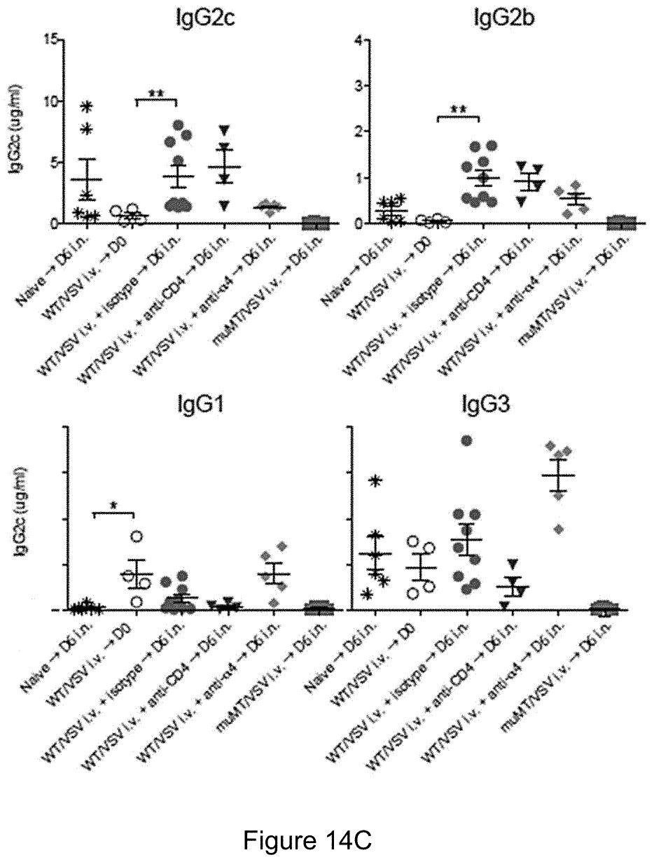

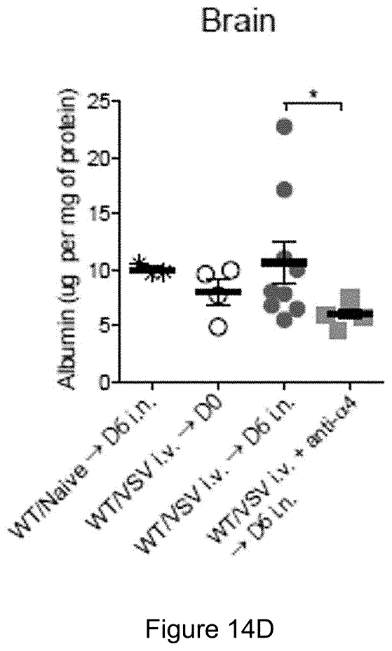

To determine whether the findings extend beyond HSV-2, the determination of antibody access to the neuronal tissue following a different neurotropic virus, vesicular stomatitis virus (VSV), a negative sense RNA virus of the Rhabdoviridae family, was investigated. Upon intranasal inoculation, VSV infects olfactory sensory neurons in the nasal mucosa and enters the CNS through the olfactory bulb (Reiss, C. S. et al., 1998, Ann. NY Acad. Sci. 855, 751-761). In contrast, intravenous infection with VSV is well tolerated, and generates robust T- and B-cell responses (FIG. 14) (Thomsen, A. R. et al., 1997, Int. Immunol. 9, 1757-1766). To determine whether antibody access to the brain requires memory CD4 T cells, mice were immunized with VSV intravenously. Five weeks later, immunized mice were challenged with VSV intranasally. Entry of VSV-specific antibodies was monitored in the brain 6 days after intranasal challenge. Consistent with the data obtained from HSV-2 infection, a striking dependence on CD4 T cells of antibody access to the brain was observed (FIG. 14B). Further, anti-α4 antibody treatment of mice immediately before intranasal VSV challenge also diminished antibody access to the brain, without impacting VSV-specific antibodies in circulation (FIG. 14C). Furthermore, it was determined that vascular permeability to the brain was dependent on α4 integrin, as antibody blockade of α4 integrin resulted in diminished albumin leakage to the brain (FIG. 14D). Taken together, these results indicate that the requirement for α4-integrin and memory CD4 T cells for antibody access applies to two distinct neurotropic viruses, HSV-2 and VSV, and suggest a general mechanism of antibody access to the immunoprivilaged tissues protected by the blood-nerve barriers.

### Example 2: A T Cell-Based Immunotherapy for CNS Viral Infections and Tumors

Access to the brain and other sites with low regeneration capacity is generally limited by tight blood-tissue endothelial layers, such as the blood-brain barrier (BBB), which restricts access to the central nervous system (CNS). While these barriers limit pathogen entry and help preserve the integrity of the tissue, they may also hinder access of protective antibodies or therapeutic molecules against infectious agents or tumors. Using a neurotropic virus infection model, experiments were conducted to grant access to the CNS of protective or therapeutic antibodies by using immune response-mediated transient BBB permeability. Primary systemic infection with vesicular stomatitis virus (VSV) led to CD4⁺ T cell-derived IFNγ-mediated transient opening of the BBB upon lethal intranasal VSV challenge, allowing entry of protective antibodies to the CNS. Mimicking local viral challenge with intranasal delivery of viral antigenic peptides alone efficiently recruited IFNγ-producing CD4⁺ T cells, triggering transient BBB permeability, protective antibody access to the CNS, and protection against a heterologous virus challenge. Intranasal delivery of T cell-specific immunogenic peptides also allowed efficient access to the CNS of anti-PD1 antibodies in a model of glioblastoma, significantly enhancing the efficacy of immunotherapy. Our results suggest that the local delivery of antigenic peptides to immune-privileged sites can transiently increase the access of therapeutic molecules or monoclonal antibodies against neurotropic pathogens, brain tumors and neurodegenerative diseases.

The materials and methods used in these experiments are now described.

Mice

Four-to-eight-week-old female C57BL/6 (CD45.2⁺), congenic C57BL/6 B6.SJL-PtprcaPep3b/BoyJ (B6.Ly5.1) (CD45.1⁺), immunoglobulin-deficient activation-induced adenosine deaminase-deficient (AID−/−) secretory IgM-deficient (sIgM−/−) double-knockout (DKO AID−/− sIgM−/−) mice and gDTII-specific-DsRed (HSV-reactive TCR Tg) transgenic mice were purchased from the National Cancer Institute and Jackson Laboratory. All efforts were made to minimize animals suffering. Mice of similar ages were randomized into control and treatment groups without any bias on parents, weight or size.

Viruses

HSV-2 strain 186syn-TK and the WT/VSV virus strain were propagated and titered on Vero cells (ATCC CCL-81). Vero cells were free of mycoplasma, as analyzed by PCR prior to use.

Viral Infection

Four-to-eight-week-old female mice were immunized subcutaneously with WT/VSV, 2×10⁶ plaque-forming units (PFU), 100 μL/mouse. For secondary challenge, immunized mice were anaesthetized with isoflurane (mixture of 30% v/v isoflurane in propylene glycerol) and inoculated intranasally with WT/VSV, 10⁷ pfu/mouse. C57/BL6 mice were immunized subcutaneously with 2×10⁶ pfu/100 ul/mouse of TK-HSV-2. Viral infection was determined by survival, weight loss and disease signs monitoring. Due to humane concerns, the animals were euthanized prior to reaching moribund state. Survival curve data are shown as percentage of survival at the end of the experiment. Viral RNA in the brain was detected by qRT-PCR.

Antibodies

Anti-CD45.2 (104), anti-CD45.1 (A20), anti-CD45 (30-F11), anti-CD3P (145-2C11), anti-CD4 (GK1.5 and RM4-5), anti-CD8a (53-6.7) were purchased from BD Biosciences, e-Bioscience or BioLegend. For intracellular staining, anti-IFNγ (XMG1.2 and R4-6A2), anti-TNFα (MP6-XT22) was purchased from BioLegend. Alexa-Fluor-488-conjugated goat anti-mouse IgG (H+L), Alexa-fluor 646 donkey anti-goat IgG (H+L), Qtracker 565 Vascular Labels and mouse IgG2a (02-6200) isotype control were purchased from Invitrogen (ThermoFischer Scientific). Anti-VSV(IE9F9) monoclonal antibody was purchased from Kerafast.

RNA Isolation and Quantitative Reverse-Transcription Polymerase Chain Reaction

To measure the virus titer, brain tissues were harvested in DMEM (1 ul of DMEM/0.2 μg of tissue) (Life Technologies, Grand Island, N.Y.) with 1% penicillin-streptomycin (Sigma). Thereafter, these tissues were homogenized by lysing matrix D (MP Biomedicals), followed by clarifying by centrifugation. RNA was isolated using TRIZOL reagent (Sigma-Aldrich) followed by RNeasy mini kit (Qiagen), according to the manufacturer's instructions. Contaminating DNA was removed using recombinant DNaseI (Roche), and cDNA was generated with iScript cDNA synthesis kit (BioRad) according to the manufacturer's instructions. Quantitative PCR was performed using SYBR green-based quantification (Qiagen). Expression of VSV RNA in brain tissues was quantitated by quantitative reverse-transcription PCR (RT-qPCR) using the following set of primers: VSV (F, ACGGCGTACTTCCAGATGG (SEQ ID NO: 3); R, CTCGGTTCAAGATCCAGGT (SEQ ID NO: 4)). Expression of target genes was normalized against housekeeping gene Hprt.

Antigenic Peptide Treatment

After 5 weeks from TK-HSV-2 infection or 24 post HSV specific CD4 T cells adoptive transfer mice were treated with gDTII peptides (HSV-2 I-Ab-restricted epitope located in glycoprotein D) or RVG-gDTII peptides (rabies viral glycoprotein associated with gDTII peptide). The animals were briefly anesthetized with isoflurane and intranasally administered the formulation 1 or 3 times (100 μg/mouse in 10 μl, 5 μl each nostril). The doses were administered subsequently within a 15 minute interval. RVG-OVA peptides (rabies virus glycoprotein associated with ovalbumin) were used as control at same concentration and dose. RVG-gDT peptides (YTIWMPENPRPGTPCDIFTNSRGKRASNGGGGCCIPPNWHIPSIQDA; SEQ ID NO:89) and gDT peptides (gD315-327, IPPNWHIPSIQDA; SEQ ID NO:90) were used as antigenic peptides for all experiments.

Isolation of Leukocytes from Neuronal Tissues

Infected mice were anesthetized and perfused intracardially with sterile ice-cold PBS. Brain tissues were collected and harvested in PBS and homogenized followed by collagenase digestion in 1 mg/mL collagenase D (Roche) and 30 μg/mL DNase I (Sigma-Aldrich) at 37° C. for 30 minutes. The resulting cells were filtered through a 100-μm filter, and further isolated using Percoll density gradient centrifugation. The cells preparation was second filtered through a 70-μm filter, washed and harvested for further stimulation and analysis.

Stimulation of Lymphocytes and Flow Cytometry

Preparation of cell suspensions from neuronal tissues were described previously. Splenocytes were homogenized and filtered through a 70-μm filter, washed and treated with ACK lysis buffer (5 mL of ACK/spleen for 1 minute). Cells were pretreated with anti-CD16/32 antibody (2.4G2) to block Fc receptors, and stained with the surface antibodies. To stain intracellular cytokines and detect HSV-2-specific CD4+ T cells or VSV-specific CD4+ T cells (CD45.1+ or CD45.2+), single cell suspensions from brain tissues of TK-HSV-2 immunized mice or VSV immunized mice were stimulated in the presence of 5 μg/ml Brefeldin A with naive splenocytes (CD45.1+CD45.2+) loaded with HSV-2 antigen (0.5 pfu equivalent per cell) for 10-12 hours. For unspecific stimulation, brain cells isolated from gDTII-transferred recipient mice were incubated with phorbol 12-myristate 13-acetate (PMA) (20 ng/mL) (Sigma-Aldrich) and ionomycin (4 μg/mL) (Merck Milipore, Billerica, Mass.) in the presence of Brefeldin A X1 (eBioscience, San Diego, Calif.) at 37° C. and 5% CO₂ for 4 hours. Cells were surface-stained with anti-CD3 (145-2C11), anti-CD4 (GK1.5 and RM4-5) and anti-CD8a (53-6.7) for 30 minutes and then fixed and permeabilized using a Cytofix/Cytoperm Fixation/Permeabilization kit (BD Biosciences) according to the manufacturer's instructions. These cells were intracellular stained with anti-IFNγ (XMG1.2) and anti-TNFα for 1 hour. Multiparameter analyses were carried out on the LSR II flow cytometer (Becton Dickinson) and data were analyzed using the FlowJo software (Tree Star Inc., Ashland, Oreg.).

In Vivo Treatment with Anti-VSV Monoclonal Antibodies and Passive Sera Administration.

DKO AID−/− sIgM−/− mice were injected intraperitoneally with 5 ug/500 ul/mouse of VSV mAb at days −1, 1 and 3 before/after intranasal VSV challenge.

An IgG2a isotype control antibody was used as a control. For obtention of VSV immune serum, VSV-immunized mice were bled at 6 days post-infection. The obtained sera were pooled and stored at −80° C. Naive serum obtained from uninfected C57BL/6 donor mice was used as a control. Prior to transfer experiments, serum was heated at 56° C. for 30 min. For passive serum transfer experiments, VSV-immune serum or naive serum (500 L) was transferred to C57BL/6 recipient mice by intraperitoneal route at days −1, 1 and 3 before/after VSV challenge. For the heterologous challenge, TK-HSV-2-immunized mice or gDTII transferred mice were injected intraperitonially with 5 μg/500 μl/mouse of VSV mAb at days 1, 3 and 5 after antigenic peptides treatment followed by intranasal VSV challenge on day 7.

Adoptive Transfer of HSV-Specific gDTII CD4+ T Cells

gDTII-specific-DsRed transgenic mice were euthanized and splenocytes were homogenized followed by red blood cell lysis in 5 mL of ACK lysing buffer/spleen for 1 min. CD4+ T cells were isolated from spleens using EasySep Mouse CD4+ T cell Isolation Kit (STEMCELL Technologies) according to the manufacturer's instructions. Then, isolated CD4+ T cells were intravenously (retro-orbital) transferred at the concentration of 10⁶ cells into C57BL/6 recipient mice. Mice were treated with antigenic peptides 24 hours after gDTII-specific cell transfer.

Tumor Injection

Mice were immunized with HSV-TK subcutaneously (s.c.) and implanted intracranially with 50,000 tumor cells in the striatum five weeks later. Six days after tumor implantation, mice were treated with gDTII peptides or OVA peptides. Experimental and control mice received anti-PD1 antibodies or isotype antibodies (200 ug/mice) on days 9 and 11 post tumor implantation. Mice were monitored for survival and tumors were imaged using IVIS luminescence imaging on days 16 and 23.

Albumin Elisa

Infected mice were deeply anesthetized by intraperitoneal injection of ketamine and xylazine, using 100 mg/kg of ketamine 10% and 20 mg/kg of xylazine 2% and then transcardially perfused with sterile ice-cold phosphate-buffered saline (PBS). Brain tissues were collected at the indicated days after infection or peptides treatment. Tissue homogenates were diluted 1:500 and albumin ELISA (Genway) was performed according to the manufacturer's instructions.

Measurement of Virus-Specific Igs and Total Igs in Tissue Homogenates

Animals were deeply anesthetized with xylazine/ketamine and then transcardially perfused with ice-cold PBS at the indicated days after viral infection or peptides treatment. For virus-specific IgG measurement a ninety-six-well EIA/RIA plate was coated with 100 μl of heat-inactivated purified VSV (5×10⁵ pfu equivalent per 100 l) in carbonate buffer (pH 9.5) and then incubated overnight at 4° C. On the following day, these plates were washed with PBS-Tween 20 and blocked 2 hours with 5% FBS in PBS. Brain tissue samples were then diluted, plated and incubate for at least four hours at room temperature. After washing in PBS-Tween 20, HRP-conjugated anti-mouse IgG or IgG2b (SouthernBiotech) was added in the wells for 1 hour, followed by washing and adding TMB solution (eBioscience). Reactions were stopped with 1N H2SO4 and absorbance was measured at 450 nm. The sample Ab titers were defined by using Ig standard (C57BL/6 Mouse Immunoglobulin Panel; SouthernBiotech) or mouse IgG2b (HOPC-1; SouthernBiotech). For total IgG measurement tissue homogenates were serial diluted and total IgG ELISA (Genway) was performed according to the manufacturer's instructions.

Vascular Permeability Assays

TK-HSV-2 immunized mice were intravenously injected (retro-orbital) with 100 μl of 5 mg/ml Oregon Green 488-conjugated dextran (70 kDa, D7173, Thermo Fisher Scientific, MA) in PBS. After 1 hour, brain tissues were carefully collected and then fixed with 4% paraformaldehyde in PBS overnight, and cut frozen sections (8 μm in thickness) for immunohistochemical analysis.

Immunofluorescence Staining

For tissues staining, frozen sections of brains tissue were cut (8 m in thickness), stained and fixed. These tissues were stained with the Abs (anti-CD4 [H129.19], anti-MHC class II [M5/114.15.2] anti-VCAM-1 [429/MVCAM.A], anti-CD31 [390 and MEC13.3], anti-Ly6G [1A8], anti-CD11b [M1/70] and anti-mouse albumin [Goat pAb/BETHYL Laboratories Inc. TX]). Stained slides were washed and incubated with DAPI, and mounted with Fluoromount-G (SouthernBiotech). Images were captured using a 10× or 40× objective lens from fluorescence microscopy (BX51; Olympus).

Statistical Analysis

Survival curves were analyzed using the log-rank test. Virus titer were analyzed using two-way analysis of variance (ANOVA). For other data, normally distributed continuous variable comparisons were performed using two-tailed Student's t test to compute the significance between the groups. All tests were performed on GraphPad Prism software. For comparison of two nonparametric datasets, the Mann-Whitney U-test was used. (*) p≤0.05; (**) p≤0.01; (***) p≤0.001; (****) p≤0.0001; not significant (ns). Values for all measurements are expressed as mean or mean±standard deviation. Mouse experiments were performed with groups of 3-18 mice. Each experiment was usually repeated two or three times.

The results of these experiments are now described

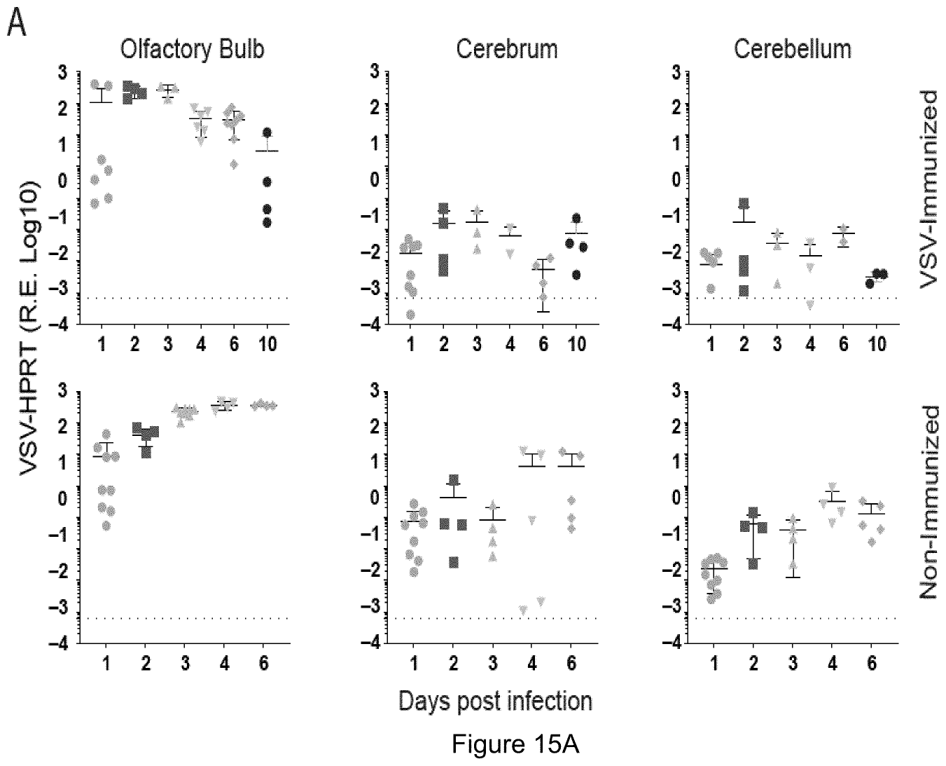

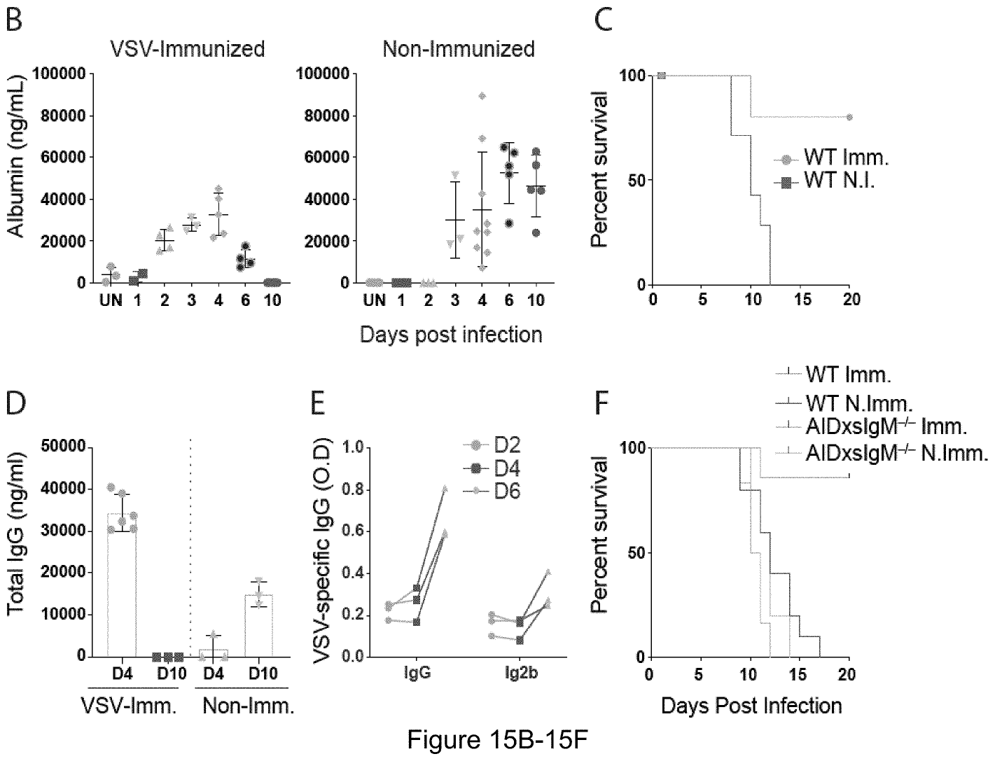

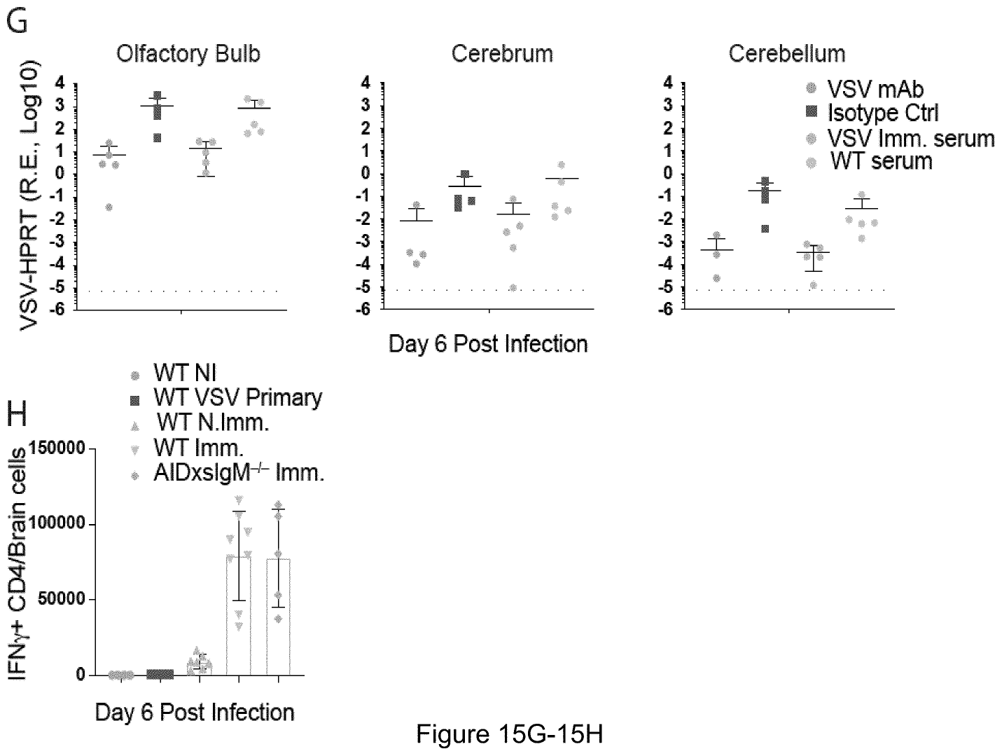

It was first assessed whether local viral challenge in immunized animals modulates brain BBB permeability, and whether CNS viral clearance is a potentially relevant strategy for modulation of CNS access of immune cells or mediators. Wild-type mice were s.c. infected with a low dose of VSV and challenged five weeks later with a lethal dose of VSV i.n., monitoring viral load during a 10-day infection period. While viral RNA was detected in the olfactory bulb of both groups, as expected after i.n. delivery, non-immunized mice showed significantly higher levels; low viral RNA was observed in the cerebrum and cerebellum of challenged mice (FIG. 15A). To investigate whether the immunization strategy, followed by local viral challenge, leads to changes in BBB permeability, serum albumin was measured in the brain of immunized and control groups. Intranasal reinfection of VSV-immunized mice resulted in transient BBB permeability while non-immunized mice showed progressive BBB leakage (FIG. 15B). A vascular disruption in the brain was detected from days 2 to 6 post intranasal challenge in immunized mice with a peak on day 4 post intranasal challenge, but the integrity of the BBB was restored as animals recovered from the infection (FIG. 15C). In contrast, BBB permeability progressed in non-immunized mice according to their viral load; these mice succumbed to viral infection between days 8 and 12 post intranasal challenge (FIG. 15C). Without being bound by theory, it was concluded that systemic viral immunization resulted in protection to local CNS viral challenge, and resistance was correlated to a transient BBB permeability.

The enhanced clearance and reduced viral load observed upon i.n. challenge in VSV-immunized mice suggested a possible transient BBB permeability in the resistance mechanisms. To determine whether the transient BBB permeability in VSV vaccinated mice enhances resistance by increasing access of protective antibody to the brain, antibody entry in the neuronal tissue was measured during VSV challenge. Consistent with the timing of BBB permeability, a significant increase in total IgG levels was detected in the brain tissue on days 4 and 6, but not on day 10 post-challenge (FIG. 15D). Accordingly, increased VSV-specific antibodies were also detected on day 2, 4 and 6 post-challenge in immunized mice (FIG. 15E). The peak of IgG antibodies in VSV-immunized mice was on day 4, while non-immunized mice showed increased IgG levels on day 10; hence in both groups infiltrating antibodies correlated to the peak of BBB permeability, however the late increase in non-immunized mice was not sufficient to confer protection (FIG. 15C). To directly address whether the increase in antibodies levels was necessary for viral clearance, activation-induced adenosine deaminase-deficient (AID−/−) and secretory IgM-deficient (sIgM−/−) double-knockout mice (AID sIgM DKO) were subjected to the VSV immunization-challenge strategy. In contrast to wild-type mice, AIDxsIgM DKO immunized mice succumbed to VSV intranasal challenge between 9- and 12-days post infection (FIG. 15F). Consistent with a protective role for anti-VSV antibodies, transfer of anti-VSV monoclonal antibody (mAb), or anti-VSV immune sera protected AIDxsIgM DKO immunized mice, when compared to mice receiving isotype control antibody or WT-serum (FIG. 15G). These data suggest that antibodies are essential to viral clearance of VSV in the CNS upon local challenge.

Using a mouse model of genital herpes infection, it was previously demonstrated that memory virus-specific CD4+ T cells migrate to the dorsal root ganglia (DRG) and spinal cord, secreting interferon (IFN)γ that in turn mediate local increase in vascular permeability, enabling antibody access for viral control (Iijima and Iwasaki, 2016, Nature, 533:552-556). To investigate whether antibody access to brain tissue is associated with local recruitment of memory CD4 T cells, T cell populations were analyzed at day 6 post infection of VSV-immunized or non-immunized mice. Notably, IFNγ-producing CD4 T cells were only detected in VSV immunized mice (FIG. 15H). Local IFNγ-producing CD4 T were also detected in AIDxsIgM DKO VSV-immunized mice, further indicating that these mice only lacked the downstream effector, antibody-mediated mechanisms of protection (FIG. 15H). These results reinforce the possibility that protection conferred by VSV-immunization is mediated by locally recruited, virus-specific IFNγ-producing CD4 T cells (FIG. 14D).

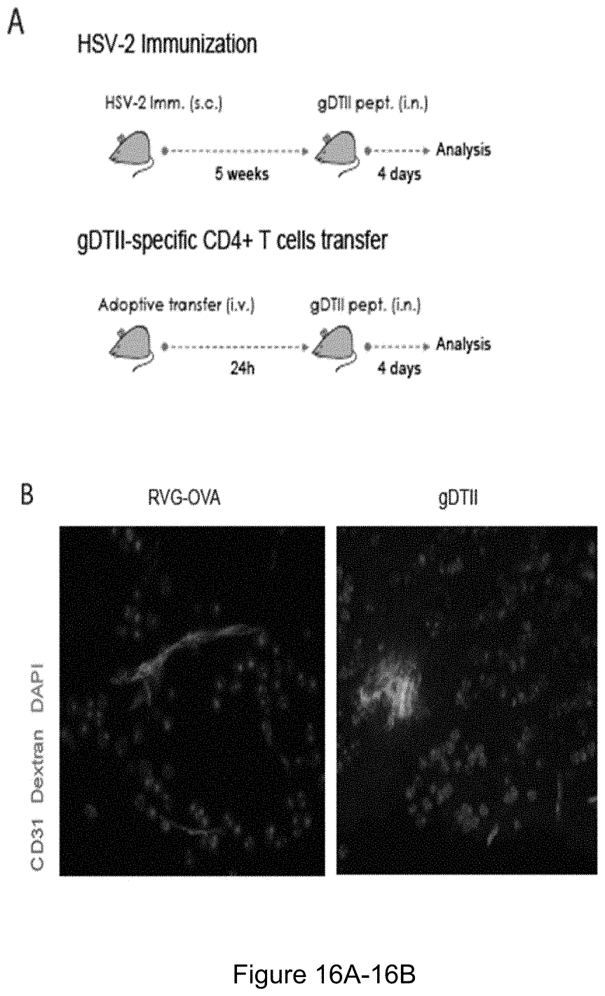

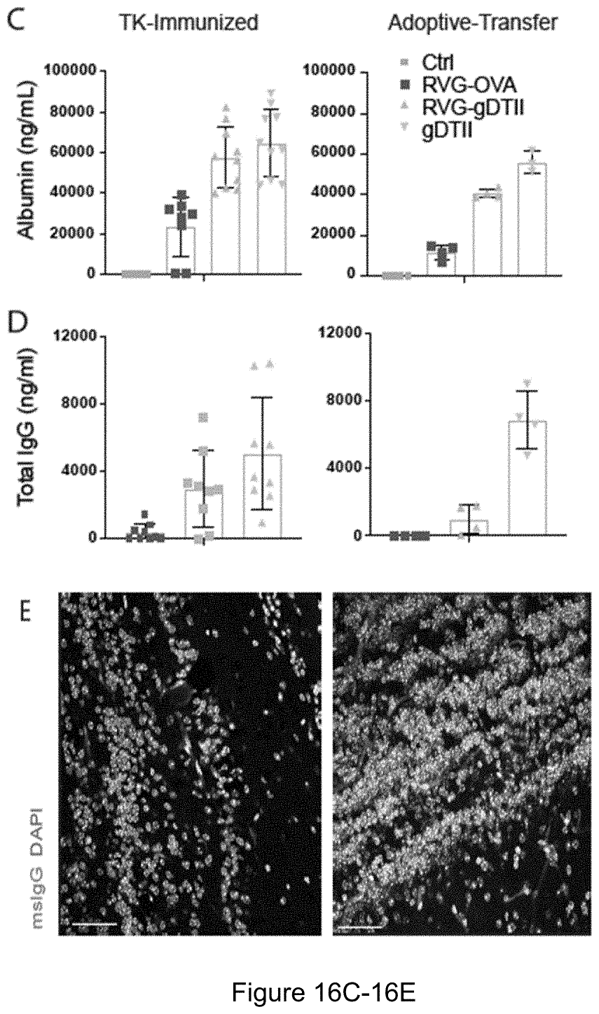

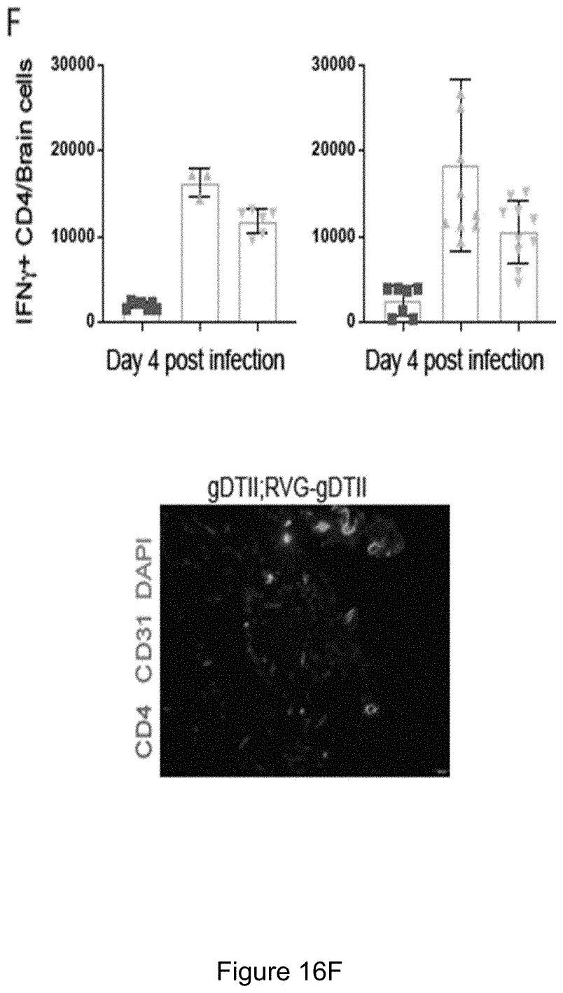

After establishing an infection/local challenge-based model that transiently opens BBB for protective antibody-access, a therapeutic strategy of local antibody delivery to the CNS was developed. The strategy first used a mouse model of genital herpes (HSV-2), associated with local i.n. delivery of viral antigenic peptides as an immunotherapeutic platform. Two complementary strategies were used for the development of a HSV-2-specific memory CD4+ T cell response (FIG. 16A). Mice were immunized with HSV-TK subcutaneously (s.c.) or received an adoptive transfer of HSV-specific CD4 T cells from gDTII-specific DsRed (HSV-reactive TCR Tg) transgenic mice. Five weeks post HSV immunization or 24 hours post HSV-reactive TCR Tg T cell transfer, HSV-immunized mice were treated with 1 or 3 doses of gDTII peptides, a HSV-2 I-Ab-restricted epitope located in the glycoprotein D or with RVG-gDTII peptides (rabies viral glycoprotein associated with gDTII peptide). Rabies virus glycoprotein associated with ovalbumin peptides (RVG-OVA) were used as control immunogen. Similar to what was observed using live virus challenge, i.n. delivery of TCR-specific viral antigenic peptides to immunized mice resulted in BBB permeability on day 4, which allowed antibody access to the CNS (FIG. 16B through FIG. 16E). Additionally, treatment with antigenic peptides induced the recruitment of IFNγ-producing polyclonal and gDTII TCR transgenic CD4+T to the brain in mice that received i.n. RVG-gDTII or gDTII, respectively (FIG. 16F). Without being bound by theory, it was concluded that local delivery of TCR-specific viral antigenic peptides may be used as a therapeutic strategy to control CNS virus infection.

Next, whether IFNγ-producing memory CD4+ T cells also coordinated the increase in BBB permeability and antibody access to the CNS after intranasal delivery of viral antigenic-peptides was evaluated. gDTII specific T cells were adoptively transferred and stimulated using antigenic peptides. Preferential BBB permeability was seen when antigenic peptides were given with adoptively transferred CD4 T cells and not when control peptides were given (FIG. 16C).

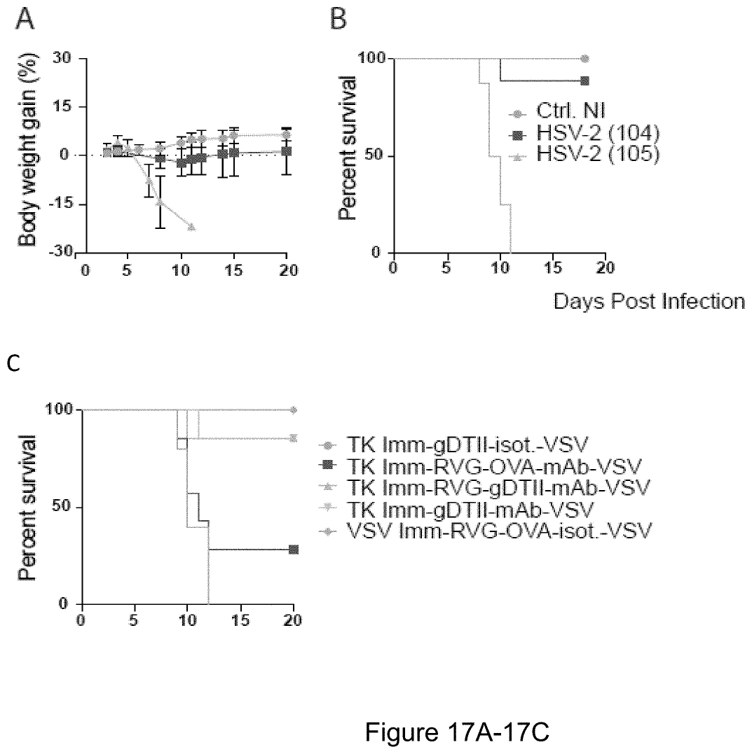

Next, the efficacy of the therapeutic strategy of local antigenic peptide delivery to the neuronal tissues for lethal heterologous infection was evaluated. Mice were immunized with TK-HSV-2, and immunogenic peptides were administered. At days 1, 3 and 5 after peptides administration mice were injected intraperitoneally with 5 ug/500 ul/mouse of VSV mAb followed by intranasal VSV challenge on day 7 and survival was monitored. Mice that were previously immunized and received antigenic peptide showed preferential protection against the heterologous VSV challenge (FIG. 17C).

To investigate whether the transient BBB permeability triggered by local antigenic peptide delivery could be used to increase the efficacy of antibodies against a heterologous neurotropic virus, HSV-2-specific peptide therapy was tested against lethal VSV infection. Similar to the experiments described in FIG. 16, mice were immunized with HSV-TK subcutaneously (s.c.) or received an adoptive transfer of naive HSV-specific CD4 T cells. Five weeks post HSV immunization or 24 hours post HSV-reactive TCR Tg T cell transfer, HSV-immunized mice were treated with gDTII peptides. Mice received a neutralizing monoclonal antibody against VSV, or isotype control antibodies, in three doses during the of BBB opening post HSV-2 peptide i.n. delivery, and subsequently challenged with a lethal dose of i.n. VSV. Monoclonal anti-VSV antibodies prevented virus spread and lethality in HSV-2 immunized mice that received HSV-2 i.n. peptide (FIG. 17D and FIG. 17E). These results suggest that local antigenic peptide delivery can effectively be used at will for protection against heterologous infection.

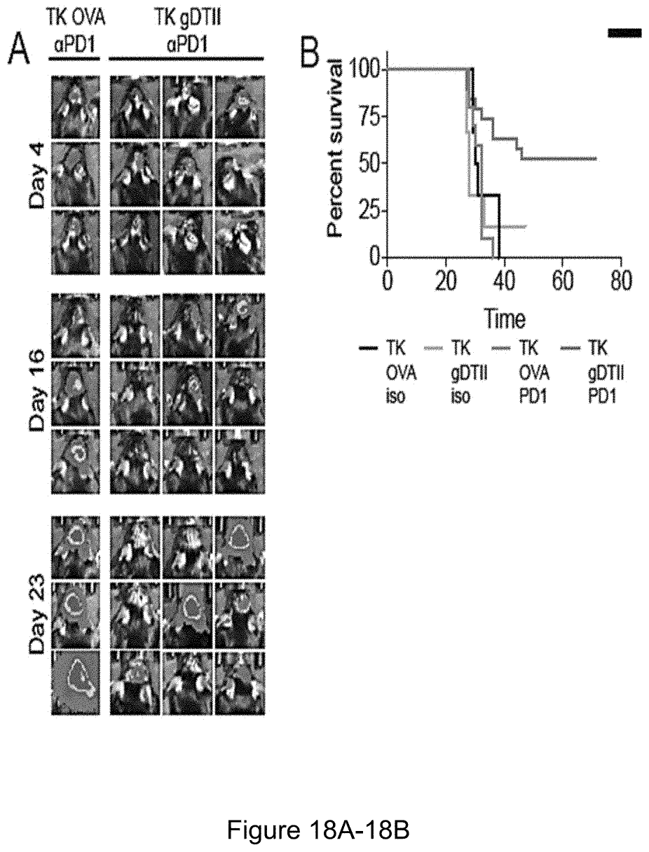

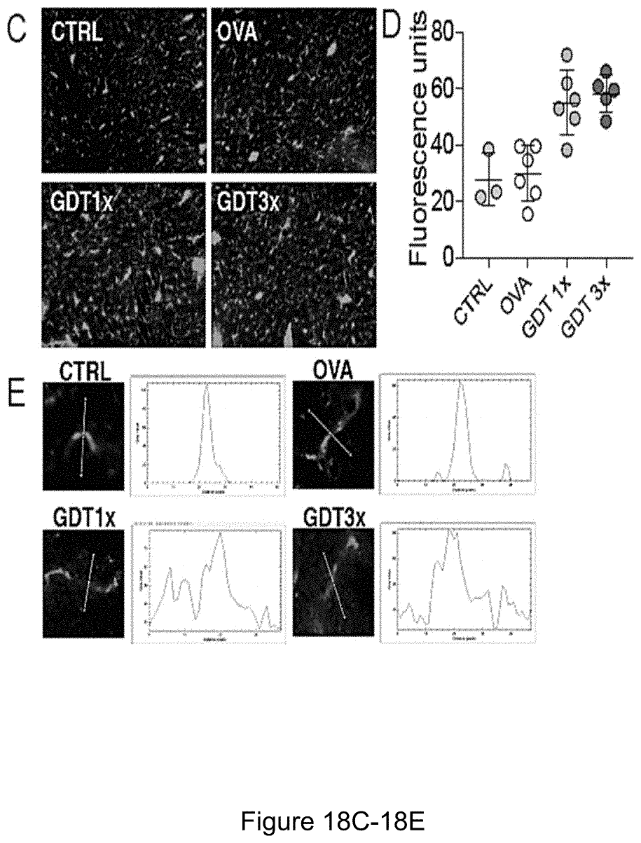

Finally, whether the transient BBB permeability could be used to increase access to the CNS of antibodies used for checkpoint blockade immunotherapy we evaluated. HSV-2-specific peptide therapy was tested in a model of glioblastoma. Mice were immunized with HSV-TK subcutaneously (s.c.) and implanted intracranially with 50,000 tumor cells five weeks later. Six days after tumor implantation, mice were treated with gDTII peptides or OVA peptides. Experimental and control mice received anti-PD1 antibodies or isotype antibodies on days 9 and 11 post tumor implantation. Tumor size was measured at day 4 to normalize for variance and subsequently on days 16 and 23 using luminescence (FIG. 18A). Anti-PD1 antibody treatment was not effective as a monotherapy in mice that did not receive antigenic peptide therapy (FIG. 18B). However, anti-PD1 therapy prevented tumor growth and significantly improved survival in mice with HSV-2 immunization and HSV-2 i.n. peptide stimulation (FIG. 18B). The antigenic peptide strategy provides a novel method of amplifying checkpoint inhibitor therapies by increasing the effective dose in the central nervous system by bypassing the BBB.

Together, these results suggest that the use of antigenic peptides could potentially lead to a new therapeutic platform for monoclonal antibody or drug delivery to combat neurotropic pathogens and brain tumors.

### Example 3: CD4⁺ T Cell Therapy for Drug Delivery Through the BBB

Brain Metastases are difficult to treat because either effective therapies cannot cross the BBB or cannot reach adequate concentrations in the microtumor environment. Brain metastasis portends high mortality with a 8.1% survival rate at 2 years and a 2.4% survival rate at 5 years. Neurosurgical excision and radiotherapy are not possible or sustainable for some patients.

Patient or tumor-specific peptides have been developed to activate an adaptive immune response. This enables transient and defined access of drugs and biologics to the CNS. This technology can allow delivery any drugs or biologics to the CNS.

CNS antigen-specific CD4⁺ T cells could mediate BBB opening. CD4⁺ T cells enter the perivascular space in the postcapillary venule, and are stimulated by perivascular antigen-presenting cells (APCs) that present viral antigen and stimulate the secretion IFN-7. IFN-7 acts on vascular ECs and downregulates tight junction proteins.

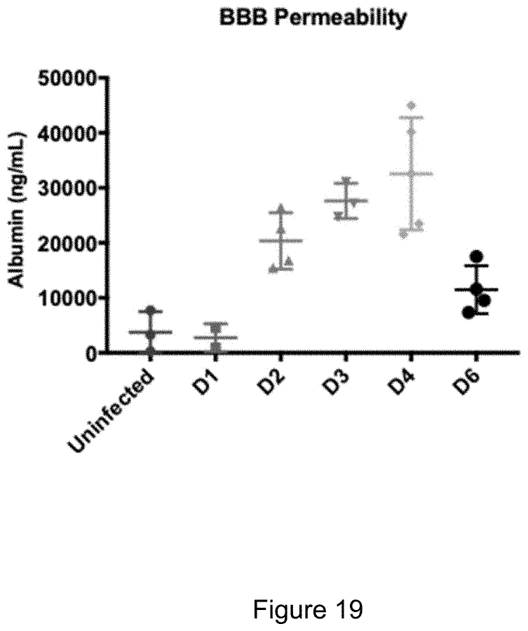

Intranasal delivery of MHC Class II peptides can function to open up the BBB (FIG. 19). The peptides will follow olfactory nerves to enter the CNS to stimulate CD4 T cells. CD4 T cells produce interferon gamma to open up the BBB for a few days. Stimulation of T cells by intranasal peptide (IN) enables checkpoint inhibitor biologics to access brain tissue and treat the tumor. Pre-existing CD4 T cells can be leveraged. In a metastatic Glioblastoma model, survival is dramatically improved by co-administration of IN and checkpoint inhibitor biologics. RVG-gDT peptides (YTIWMPENPRPGTPCDIFTNSRGKRASNGGGGCCIPPNWIHIPSIQDA; SEQ ID NO:89) and gDT peptides (gD315-327, IPPNWHIPSIQDA; SEQ ID NO:90) were used as antigenic peptides for these experiments.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for treating or preventing a disease or disorder of the brain, central nervous system or spinal cord in a subject in need thereof, comprising:
a) administering an immunogenic agent to induce an immune response, thereby inducing permeability of the blood brain barrier (BBB) in the subject; and
b) administering at least one therapeutic agent for the treatment of the disease or disorder.

2. The method of claim 1, wherein the immunogenic agent is an antigenic protein or peptide for inducing a CD4 T cell immune response.

3. The method of claim 2, wherein the immunogenic agent comprises an antigenic MHC Class II peptide.

4. The method of claim 2, wherein the immunogenic agent comprises a peptide selected from the group consisting of SEQ ID NO:5 to SEQ ID NO:90.

5. The method of claim 1, wherein at least one therapeutic agent comprises an inhibitor of an immune checkpoint protein.

6. The method of claim 5, wherein the immune checkpoint protein is selected from the group consisting of PD-1, PDL-1 CTLA-4, LAG-3, TIM-3, TIGIT and CEACAM1.

7. The method of claim 6, wherein the inhibitor is selected from the group consisting of ipilimumab, nivolumab, pembrolizumab, pidilizumab, atezolizumab, BMS-986016, BMS-936559, MPDL3280A, MDX1105-01, MEDI4736, TSR-022, CM-24 and MK-3475.

8. The method of claim 1, wherein the disease or disorder comprises a pathogen-mediated infection selected from the group consisting of: a viral infection, a bacterial infection, a fungal infection, a protozoan infection, a prion infection, and a helminth infection.

9. The method of claim 1, wherein the method treats or prevents infection-associated inflammation.

10. The method of claim 1, wherein the method treats or prevents an infection-associated condition selected from the group consisting of: encephalitis, meningitis, meningoencephalitis, epidural abscess, subdural abscess, brain abscess, and progressive multifocal leukoencephalopathy (PML).

11. The method of claim 1, wherein the method treats or prevents cancer.

12. The method of claim 11, wherein the therapeutic agent comprises an antibody or antibody fragment that specifically binds a tumor-specific or tumor-associated antigen.

13. A composition for treating or preventing a disease or disorder of the brain, central nervous system or spinal cord in a subject in need thereof, comprising:
a) an antigenic protein or peptide to induce a CD-4 T cell immune response in the subject, thereby inducing permeability of the BBB; and
b) at least one therapeutic agent for the treatment of the disease or disorder.

14. The composition of claim 13, wherein the antigenic protein or peptide comprises an antigenic MHC Class II peptide.

15. The composition of claim 14, wherein the antigenic protein or peptide is selected from the group consisting of SEQ ID NO:5 to SEQ ID NO:90.

16. The composition of claim 14, wherein at least one therapeutic agent comprises an inhibitor of an immune checkpoint protein.

17. The composition of claim 16, wherein the immune checkpoint protein is selected from the group consisting of PD-1, PDL-1 CTLA-4, LAG-3, TIM-3, TIGIT and CEACAM1.

18. The composition of claim 16, wherein the inhibitor is selected from the group consisting of ipilimumab, nivolumab, pembrolizumab, pidilizumab, atezolizumab, BMS-986016, BMS-936559, MPDL3280A, MDX1105-01, MEDI4736, TSR-022, CM-24 and MK-3475.

19. The composition of claim 14, wherein the therapeutic agent comprises an antibody or antibody fragment that binds to an antigen associated with the disease or disorder.

20. The composition of claim 14, wherein the disease or disorder is selected from the group consisting of a viral infection, a bacterial infection, a fungal infection, a protozoan infection, a prion infection, a helminth infection, encephalitis, meningitis, meningoencephalitis, epidural abscess, subdural abscess, brain abscess, progressive multifocal leukoencephalopathy (PML), and cancer.
